(19) United States
(12) Patent Application Publication
(10) Pub. No.: US 2025/0387791 A1
(43) Pub. Date: Dec. 25, 2025

(54) MANIFOLD DESIGNS FOR MODULATING FLOW DISTRIBUTION

(71) Applicant: EMD Millipore Corporation, Burlington, MA (US)

(72) Inventors: Heejin Lee, Bedford, MA (US); Josephine Bowen, Nashua, NH (US); Benjamin Cacace, Tewksbury, MA (US); Wenying Quan, Bedford, MA (US); John Paul Amara, Reading, MA (US)

(21) Appl. No.: 19/236,269

(22) Filed: Jun. 12, 2025

Related U.S. Application Data
(60) Provisional application No. 63/661,796, filed on Jun. 19, 2024.

(51) Int. Cl. B01L 3/00

(52) U.S. Cl. CPC B01L 3/563; B01L 3/561; B01L 2300/0681

(57) ABSTRACT

A series of flow manifolds are provided, where each is configured to achieve a uniform or near uniform flow distribution among multiple combined filtration devices, such as closed depth filter devices, pre-filters, or any other filtration devices that combine to increase an effective filter area. One flow manifold is configured to utilize flow restrictor elements in each branch, where the flow restrictor elements each have a relatively large flow resistance compared with that of filter devices. The flow restrictors may be selectively used on demand during a bioprocessing process. Another flow manifold may be a bifurcated manifold where the main conduit, primary branch conduit, and secondary branch conduits have differing internal diameters. Yet another flow manifold may utilize flow restrictors disposed within hose barbs of the filtration devices.

### CROSS-REFERENCE TO RELATED APPLICATIONS

This present application claims priority to U.S. Provisional Patent Application Ser. No. 63/661,796, entitled “MANIFOLD DESIGNS FOR MODULATING FLOW DISTRIBUTION,” and filed on Jun. 19, 2024, the disclosure of which is incorporated herein by reference in its entirety for all purposes.

### TECHNICAL FIELD

Embodiments of the technologies disclosed herein relate to manifold designs that achieve a more uniform flow distribution among multiple branches and, particularly, a more uniform flow distribution among multiple combined filtration devices (e.g., depth filter devices, pre-filters, etc.) useful in bioprocessing.

### BACKGROUND OF THE INVENTION/DESCRIPTION OF RELATED ART

Filtration operations are performed in the downstream processing of biological feed streams used in the production of therapeutic biopharmaceuticals. In these operations, it is often necessary to increase the filtration area of filtration devices such as depth filters, membrane adsorbers, or virus filters in order to filter large volumes of these feed streams at the production scale. To achieve this, bioprocessing devices for filtration or purification are often manifolded to increase the effective filter area or membrane area while maintaining the modularity of each device. Examples of such devices are depth filters (including Millistak+pod depth filters), tangential flow filtration (TFF) devices or capsules, and anion exchange membrane devices. These devices may be modular and may have sterile connectors, which can be connected to external manifold tube sets. The ability to be connected by sterile connectors to external manifolds is an important aspect to enable closed processing. When multiple devices are combined with one another via a manifold, it is important to ensure flow uniformity across the combined devices. Non-uniform flow distribution across the devices can be caused by variability in flow resistance of each device that is connected to the manifold (e.g., lot-to-lot variability in the devices) and/or the design of the manifold itself. Potential issues caused by a non-uniform flow distribution include under- or over-loading of the filter devices that are manifolded, and a non-uniform flushing among the filter devices that are manifolded. For example, a uniform pre-use flushing is important for depth filters, which require pre-use flushing with water or buffer solutions (i.e., non-clogging feed stream). With traditional U-type or Z-type manifold configurations, the filter devices are subjected to an uneven flow distribution, which means there is also a non-uniform pre-use flushing of the filter devices when applicable (e.g., depth filters, etc.).

Manifolding of multiple filtration devices is currently accomplished through the use of symmetric/bifurcation splitting or U-type/Z-type splitting configurations (see FIGS. 1-3). From a flow distribution perspective, bifurcation or symmetric splitting is preferred for its symmetric or naturally balanced nature of flow, but, in practice, such a symmetric manifold design may become complicated to implement as the number of branches increase. From a practical design perspective as the number of branches increase, U-type or Z-type manifolding configurations are preferred. Moreover, bifurcation or symmetric splitting manifolds have a limited number of splitting configurations, where they can only have an even number of branches (e.g., 2″, where n is an integer greater than zero).

FIGS. 4 and 5 illustrate possible U-type or Z-type manifolding examples for bioprocessing applications, where each filtration device is indicated as a rectangular “pod.” In some embodiments, the pod may represent a pod-type depth filtration device, like that illustrated in FIG. 6. FIG. 6 illustrates an example embodiment of a pod-type depth filtration device 10, and it will be apparent to one of ordinary skill in the art that the rectangular and schematic pods of FIGS. 4 and 5 may represent other variations of pod-type filtration devices. Continuing with FIG. 6, the filtration device 10 may be an assembly of a plurality of rigid filter packets 12, each of which includes one or more fluid ports 14 that provides fluid communication to one or more fluid channels formed in each packet 12. In the embodiment shown, there are ten such packets 12, but fewer or more could be used to form a filtration device 10. The filtration device 10 also includes two opposite rigid endcaps 12′ that together sandwich the packets 12 between them. The packets 12 and the filtration device 10 may be disposable single-use devices, and may be made of a suitable material that is sterilizable, such plastic, polycarbonate, or a polyolefin such as polypropylene.

In certain embodiments, a plurality of individual packets 12 may be stacked together to form the filtration device 10, and may be interconnected with one another to provide fluid communication between them through their respective fluid ports 14 such that the packets 12 operate with one another to facilitate a parallel filtration operation. A combined filtration assembly may be assembled with a plurality of packets 12 as well as a plurality of filtration devices 10 that can be interconnected to one another. The combined filtration assembly may be stored and/or transported in a rack or the like. In certain embodiments, one of the fluid ports 14 may be an inlet port for the introduction of a liquid sample into the combined filtration assembly, one or more may be an outlet port for removal of a liquid sample from the combined filtration assembly, and one or more may be a vent port for venting gas such as air from the combined filtration assembly.

One or more of the filter packets 12 may contain media, such as media suitable for depth filtration, tangential flow filtration, cross-flow filtration, etc. Exemplary depth filtration media includes diatomaceous earth, cellulose, activated carbon, polyacrylic fiber and silica, such as those sold under the Clarisolve® and Millistak+® names by MilliporeSigma.

Returning to FIGS. 4 and 5, and as previously explained, for depth filtration processes, pre-use flushing is currently performed using water or a buffer solution (i.e., non-clogging feed stream). Even with a naturally balanced or bifurcation splitting manifold configuration (like that illustrated in FIG. 1), if the flow resistance (or permeability) of one bioprocessing or filtration device (e.g., one pod) is significantly different from the rest of the devices within the manifold, it is hard to achieve a uniform flow distribution across the devices. This variation of flow resistance can come from lot-to-lot variations in filter media permeability. A non-uniform flow distribution may result in uneven flushing. More specifically, one or more of the manifolded bioprocessing or filtration devices (e.g., pod) may be under flushed, leading to the elevation of the total organic carbon (hereinafter “TOC”) extractables level in the effluent feed stream in the subsequent process.

Therefore, what is needed is a manifold design that accounts for the variations in filter media permeability of different filtration devices to enable a uniform flow distribution through the manifold. A manifold design that is able to account for the variations in filter media permeability of each of the filtration devices of the manifold results in both even flushing of the filtration devices and a uniform flow through the manifold post flushing of the filtration devices. Thus, disclosed herein is a manifold design and manifolding strategy to achieve a uniform flow distribution within a manifolded series of filter devices.

### SUMMARY OF THE INVENTION

Embodiments described herein are, flow manifolds for bioprocessing devices, apparatuses, and/or systems that are configured to achieve a uniform or near uniform flow distribution among multiple filtration devices (e.g., closed depth filter devices, pre-filters, or any other filtration devices, etc.) coupled to or disposed within/on the flow manifolds, where the multiple filtration devices are utilized together to increase an effective filter area. The disclosed flow manifolds may be utilized for flushing or non-clogging feed streams as well as clogging feed streams.

In an embodiment, a flow manifold for a bioprocessing device may include a main conduit and a plurality of branch conduits. The plurality of branch conduits may be in fluid communication with the main conduit. Moreover, each branch conduit may include a flow restrictor that is configured to evenly distribute a fluid flowing through the flow manifold among the plurality of branch conduits.

In some instances, the flow restrictor may be a tubing with a first internal diameter that is smaller than a second internal diameter of the main conduit and a third internal diameter of each of the plurality of branch conduits. In some further instances, the flow restrictor may be a pinch clamp, or an in-line flow orifice flow restrictor.

In even some further instances, the flow manifold may further include a filter coupled to each of the plurality of branch conduits, where each filter has a first flow resistance that is less than a second flow resistance of each of the flow restrictors. In some additional instances, the second flow resistance is at least three times larger than the first flow resistance.

In some even further instances, each of the filters have similar media grades. In some other instances, each of the filters have different media grades. In some additional instances, the flow manifold is a linear Z-shaped manifold. In some even further instances, the flow manifold is a linear U-shaped manifold. In yet some further instances, the flow manifold is a bifurcated manifold, the bifurcated manifold having a primary branch conduit coupled to the main conduit, and wherein the plurality of branch conduits are a plurality of secondary branch conduits coupled to the primary branch conduit.

In another embodiment, a flow manifold for a bioprocessing device may include a plurality of conduits and a plurality of filter devices. The plurality of conduits may collectively form a plurality of flow pathways. Moreover, each conduit of the plurality of conduits may have a first flow resistance. Each filter device of the plurality of filter devices may be coupled to a respective conduit. Each filter device may have a second flow resistance that is less than the first flow resistance. The flow manifold may be configured to equally distribute a fluid flowing through the flow manifold.

In some instances, each of the filters is connected to the plurality of conduits via at least one hose barb. In some further instances, the at least one hose barb is equipped with a flow restrictor disposed within an internal conduit of the hose barb.

In some additional instances, the plurality of conduits may include a main conduit, a primary branch conduit, and a plurality of secondary branch conduits. The main conduit may have a first internal diameter. The primary branch conduit may be coupled to the main conduit and may have a second internal diameter that is less than the first internal diameter. The plurality of secondary branch conduits may be coupled to the primary branch conduit. Each of the plurality of secondary branch conduits may have a third internal diameter that is less than the first internal diameter and the second internal diameter. Each of the filter devices of the plurality of filter devices may be coupled to a respective secondary branch conduit of the plurality of secondary branch conduits.

In some other instances, the plurality of conduits may include a main conduit and a plurality of branch conduits. The main conduit may have a first internal diameter. The plurality of branch conduits may be coupled to the main conduit. Each of the plurality of secondary branch conduits may have a second internal diameter that is less than the first internal diameter. Each of the filter devices of the plurality of filter devices may be coupled to a respective branch conduit of the plurality of branch conduits.

In yet some further instances, each of the plurality of filter devices may be of similar media grades having the second flow resistance. However, in some other instances, the plurality of filter devices may include a first subset and a second subset.

The first subset of the plurality of filter devices may be of a first media grade having the second flow resistance. The second subset of the plurality of filter devices may be of a second media grade having a third flow resistance that is greater than the second flow resistance but less than the first flow resistance.

In yet some other instances, the plurality of conduits may include a main conduit, a plurality of branch conduits coupled to the main conduit, and a plurality of flow restrictor conduits. Each flow restrictor conduit of the plurality of flow restrictor conduits may be coupled to a respective branch conduit of the plurality of branch conduits. Each of the plurality of flow restrictor conduits may be equipped with a flow restrictor that imparts the first flow resistance to a respective flow restrictor conduit of the plurality of flow restrictor conduits. Each branch conduit of the plurality of branch conduits may have a third flow resistance that is less than the first flow resistance and the second flow resistance. The plurality of filter devices may be coupled to the plurality of branch conduits downstream from the flow restrictor conduits.

In yet another embodiment, a flow manifold for a bioprocessing device may include a main conduit, a plurality of filter branch conduits, a plurality of flow restrictor conduits, and a plurality of flow regulators. The plurality of filter branch conduits may be in fluid communication with the main conduit. Each filter branch conduit may include a filter device. The plurality of flow restrictor conduits may each include a flow restrictor. Each flow restrictor conduit may have a first end and an opposite second end, where the first and second ends may be coupled to a respective filter branch conduit upstream of the filter device. The plurality of flow restrictors may be configured to evenly distribute a fluid flowing through the manifold among the plurality of filter branch conduits. Each flow regulator of the plurality of flow regulators may be operatively coupled to a filter branch conduit of the plurality of filter branch conduits. Each flow regulator may be configured to regulate the flow of the fluid through either the respective flow restrictor conduit or divert the flow of the fluid from the flow restrictor conduit.

In some instances, each filter has a first flow resistance that is greater than a second flow resistance of each of the flow restrictors. In some further instances, the flow manifold is a linear Z-shaped manifold or a linear U-shaped manifold. In some even further instances, the flow manifold is a bifurcated manifold.

### BRIEF DESCRIPTION OF THE DRAWINGS

The apparatuses, systems, devices, manifolds, filtration devices, modules, components, etc., presented herein may be better understood with reference to the following drawings and description. It should be understood that some elements in the figures may not necessarily be to scale and that emphasis has been placed upon illustrating the principles disclosed herein. In the figures, like-referenced numerals designate corresponding parts/steps throughout the different views.

FIG. 1 illustrates a schematic diagram of an existing symmetric or bifurcation splitting manifold design.

FIG. 2 illustrates a schematic diagram of an existing U-type or reverse flow system manifold design.

FIG. 3 illustrates a schematic diagram of an existing Z-type or parallel flow system manifold design.

FIG. 4 illustrates a schematic diagram of the existing U-type manifold design of FIG. 2 equipped with multiple filtration devices.

FIG. 5 illustrates a schematic diagram of the existing Z-type manifold design of FIG. 3 equipped with multiple filtration devices.

FIG. 6 illustrates a perspective view of a depth filtration device, according to an example embodiment.

FIG. 7A illustrates a schematic diagram of a U-type manifold design equipped with multiple filtration devices and flow restrictor elements in accordance with the present disclosure, the U-type manifold shown in a first configuration.

FIG. 7B illustrates a schematic diagram of the U-type manifold design of FIG. 7A shown in a second configuration.

FIGS. 7C-7G illustrate charts and flow simulation data that depict the effect of different sized flow restrictor elements in distributing a flow more evenly through various U-type manifold designs in accordance with FIGS. 7A and 7B.

FIG. 8A illustrates a schematic diagram of a Z-type manifold design with multiple filtration devices and flow restrictor elements in accordance with the present disclosure, the Z-type manifold configuration shown in a first configuration.

FIG. 8B illustrates a schematic diagram of the Z-type manifold design of FIG. 8A shown in a second configuration.

FIGS. 8C and 8D illustrate charts and flow simulation data that depict the effect of different sized flow restrictor elements in distributing a flow more evenly through various Z-type manifold designs in accordance with FIGS. 8A and 8B.

FIGS. 9 and 10 illustrate schematic diagrams of various embodiments of manifold tube sets with flow restrictor elements in accordance with the present disclosure.

FIGS. 11A and 11B illustrate charts and flow simulation data that depict the effect of different sized flow restrictor elements in distributing a flow more evenly through various manifold tube configurations in accordance with FIGS. 9 and 10.

FIG. 12 illustrates a schematic diagram of a manifold tube set with flow restrictor elements and where a subset of the branch conduits extend from the main conduit in an opposing direction from a second subset of the branch conduits, and in accordance with the present disclosure.

FIG. 13A illustrates a schematic diagram of a bifurcation splitting manifold design with multiple filtration devices and flow restrictor elements in accordance with the present disclosure, the bifurcation splitting manifold shown in a first configuration.

FIG. 13B illustrates a schematic diagram of the bifurcation splitting manifold design of FIG. 13A shown in a second configuration.

FIG. 14A illustrates a schematic diagram of a bifurcation splitting manifold design with flow restrictor elements and multiple filtration devices, where each of the filtration devices have approximately the same or equal flow resistances, and in accordance with the present disclosure.

FIG. 14B illustrates a graph of the volumetric flow data of the bifurcation splitting manifold design of FIG. 14A without flow restrictors.

FIG. 14C illustrates a graph of the volumetric flow data of the bifurcation splitting manifold design of FIG. 14A with flow restrictors.

FIG. 15A illustrates a schematic diagram of a bifurcation splitting manifold design with flow restrictor elements and multiple filtration devices, where at least one of the filtration devices has a flow resistance that is significantly different from the other filtration devices, and in accordance with the present disclosure.

FIG. 15B illustrates a graph of the volumetric flow data of the bifurcation splitting manifold design of FIG. 15A without flow restrictors.

FIG. 15C illustrates a graph of the volumetric flow data of the bifurcation splitting manifold design of FIG. 15A with flow restrictors.

FIG. 16 illustrates a schematic diagram of a flow restrictor element of a manifold restrictor element in accordance with the present disclosure.

FIG. 17 illustrates a schematic illustration of a filtration device connected to a manifold having an inlet conduit, an outlet conduit, and a vent conduit, where the vent conduit may be utilized with a flow restrictor for non-clogging feed streams in accordance with the present disclosure.

FIG. 18 illustrates a perspective view of a hose barb having an integrated flow restrictor in accordance with the setup illustrated in FIG. 17 and in accordance with the present disclosure.

FIG. 19A illustrates a schematic diagram of a linear manifold design with multiple filtration devices, individual outlets from each branch, and pressure transducers in accordance with the present disclosure.

FIG. 19B illustrates a schematic diagram of a linear Z-type manifold design with multiple filtration devices and pressure transducers in accordance with the present disclosure.

FIG. 19C illustrates a schematic diagram of a linear U-type manifold design with multiple filtration devices and pressure transducers in accordance with the present disclosure.

FIG. 20A illustrates a schematic diagram of a bifurcation splitting manifold design with multiple filtration devices, individual outlets from each branch, and pressure transducers in accordance with the present disclosure.

FIG. 20B illustrates a schematic diagram of a bifurcation splitting manifold design with multiple filtration devices and pressure transducers in accordance with the present disclosure.

FIGS. 21A-21J illustrate various graphs of the volumetric flow data of flushing experiments run on the linear manifold design of FIG. 19A and the bifurcation splitting manifold design of FIG. 20A, where the internal diameters of the manifold tubing and the filtration devices were varied.

FIGS. 22A-22K illustrate various graphs of the volumetric flow data of fouling experiments run on the linear Z-type manifold design of FIG. 19B, the linear U-type manifold design of FIG. 19C, and the bifurcation splitting manifold design of FIG. 20B, where the internal diameters of the manifold tubing, the filtration devices, and the feed stream are varied.

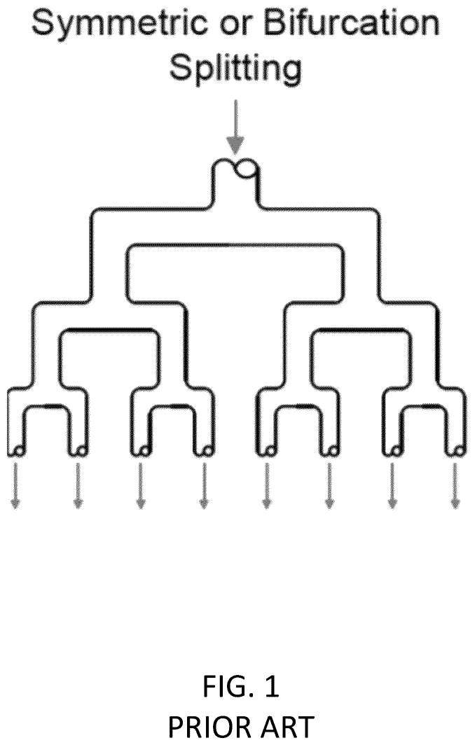

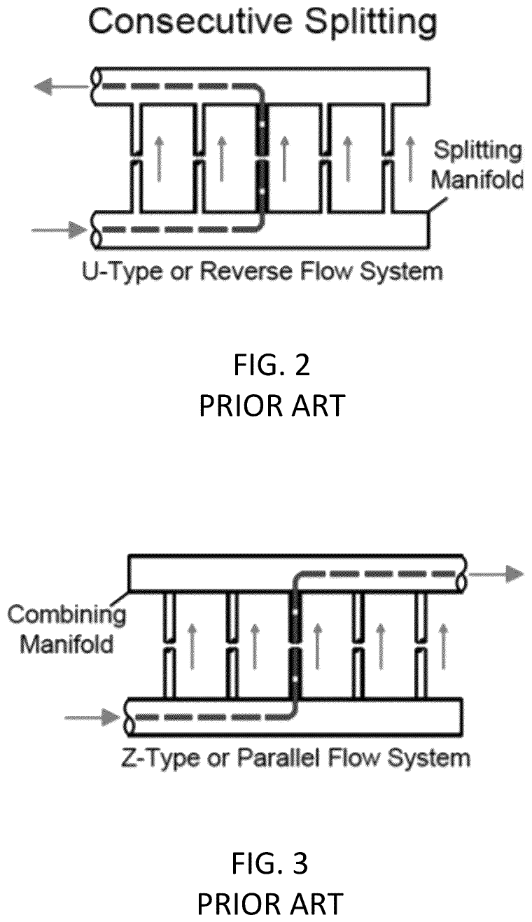

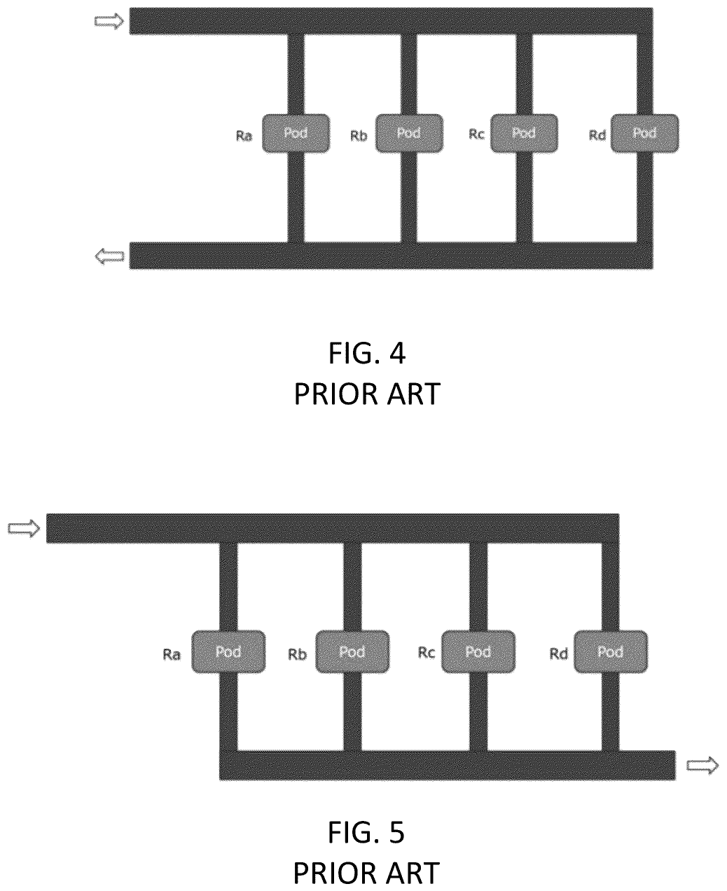

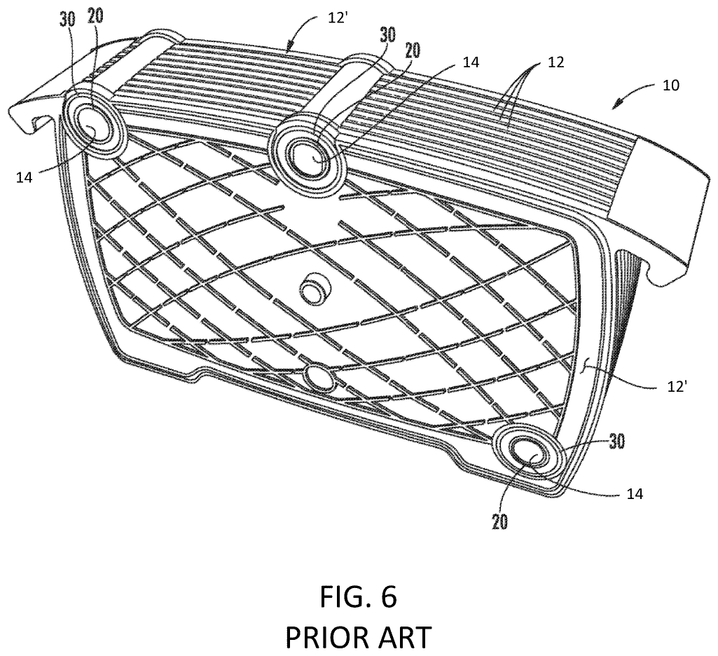

### DETAILED DESCRIPTION

Aspects of the disclosure are disclosed in the description herein. Alternate embodiments of the present disclosure and their equivalents may be devised without parting from the spirit or scope of the present disclosure. It should be noted that any discussion herein regarding “one embodiment,” “an embodiment,” “an exemplary embodiment,” and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of a given embodiment may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

Although specific terms are used in the following description for the sake of clarity, these terms are intended to refer only to the particular structure of the embodiments selected for illustration in the drawings. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2% to 10%” is inclusive of the endpoints, 2% and 10%, and all the intermediate values).

As used herein, approximating language may be applied to modify any quantitative representation that may vary without resulting in a change in the basic function to which it is related. Accordingly, a value modified by a term or terms, such as “about,” “approximately,” and “substantially,” may not be limited to the precise value specified, in some cases. The modifiers should also be considered as disclosing the range defined by the absolute values of the two endpoints. For example, the expression “from about 2 to about 4” also discloses the range “from 2 to 4.”

It should be noted that some terms used herein are relative terms. For example, the terms “upper” and “lower” are relative to each other in location, i.e., an upper component is located at a higher elevation than a lower component and should not be construed as requiring a particular orientation or location of the structure. As a further example, the terms “interior,” “exterior,” “inward,” and “outward” are relative to a center, and should not be construed as requiring a particular orientation or location of the structure.

The terms “top” and “bottom” are relative to an absolute reference, i.e., the surface of the earth. Put another way, a top location is always located at a higher elevation than a bottom location, toward the surface of the earth.

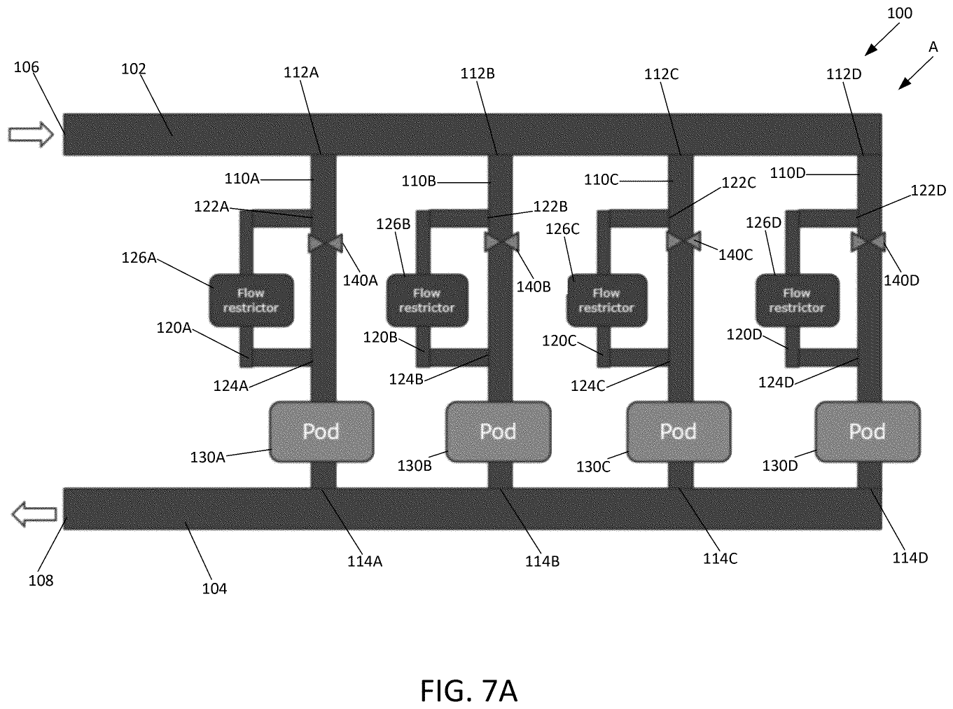

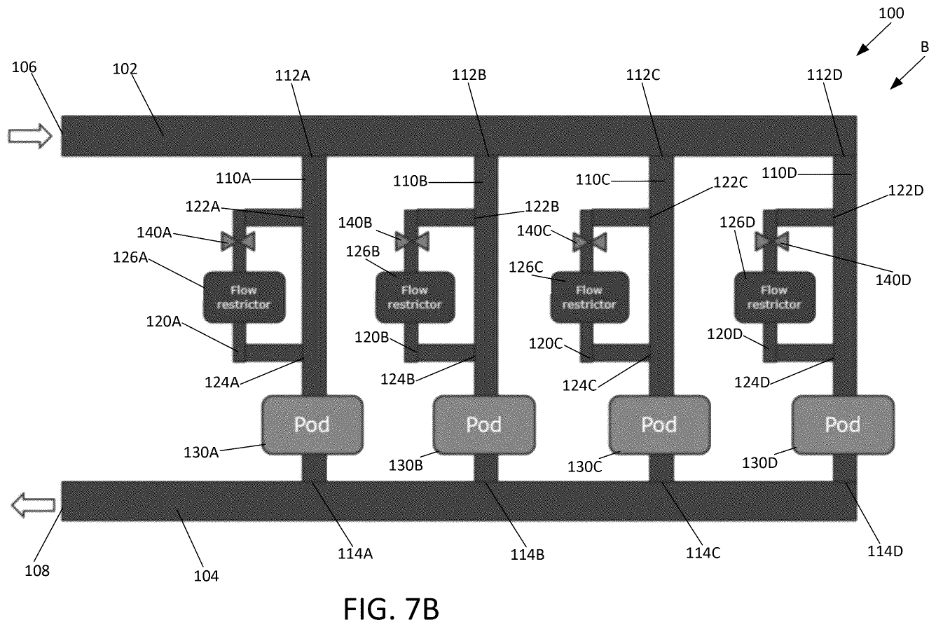

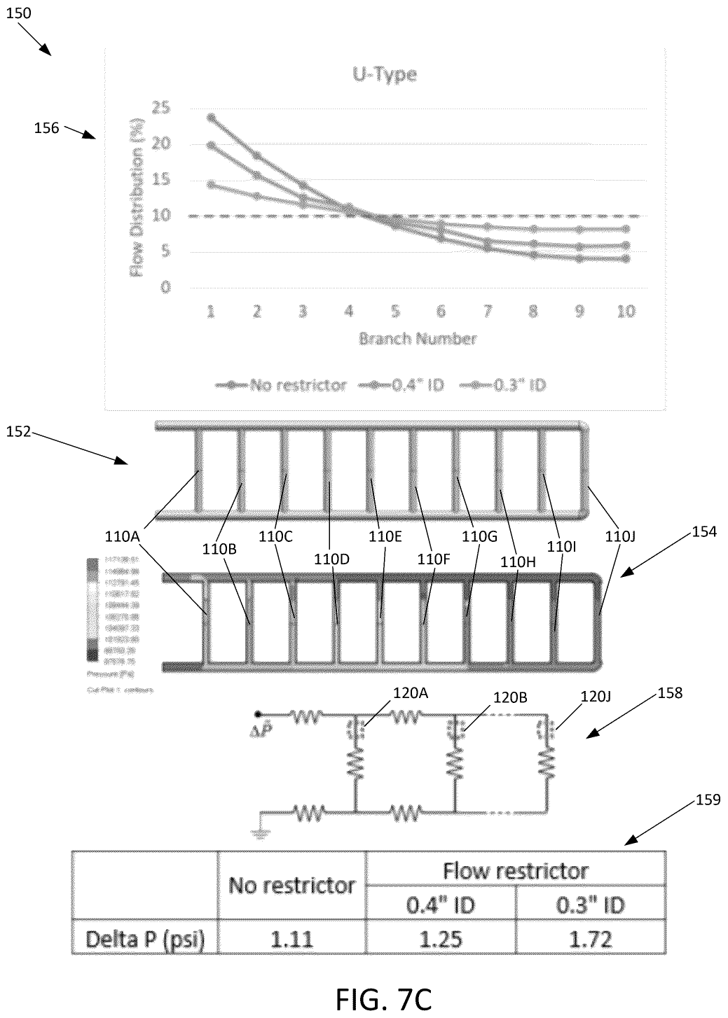

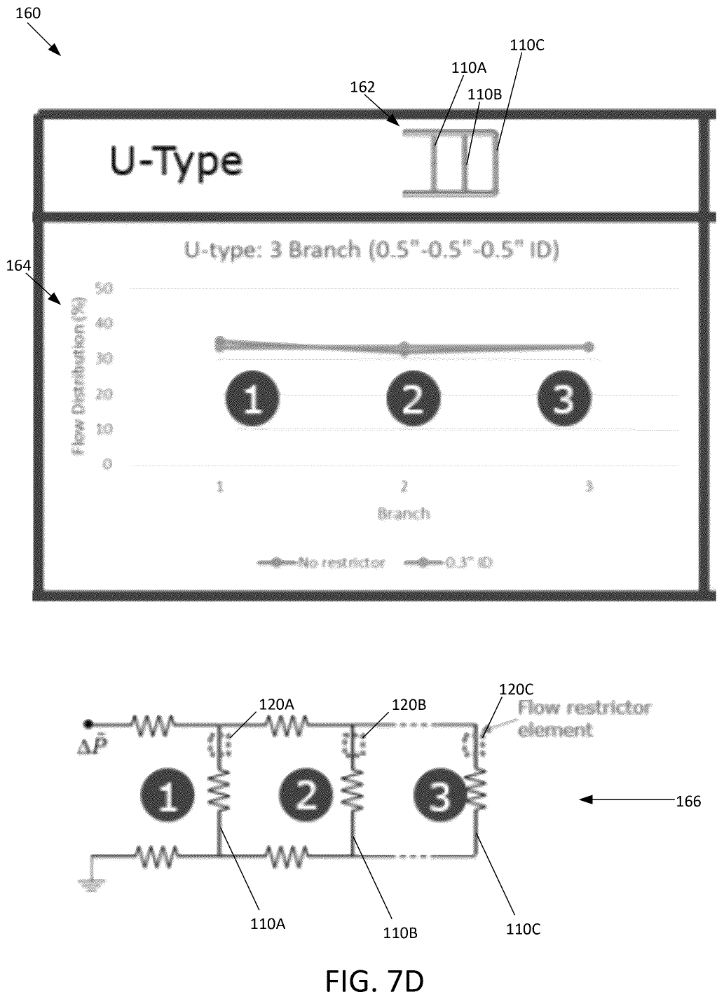

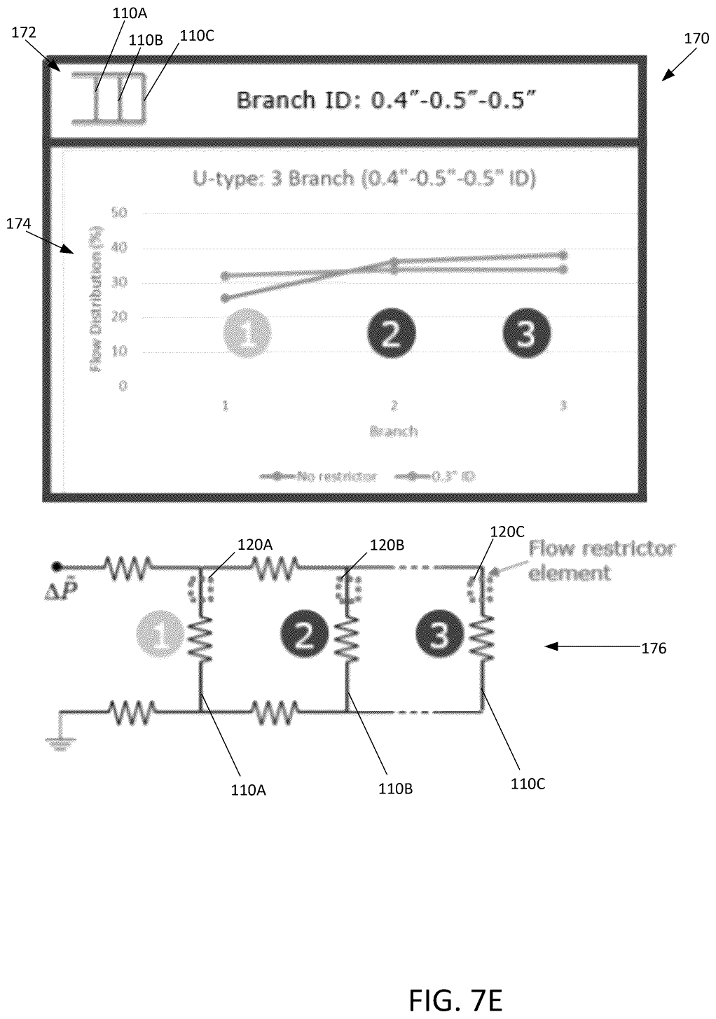

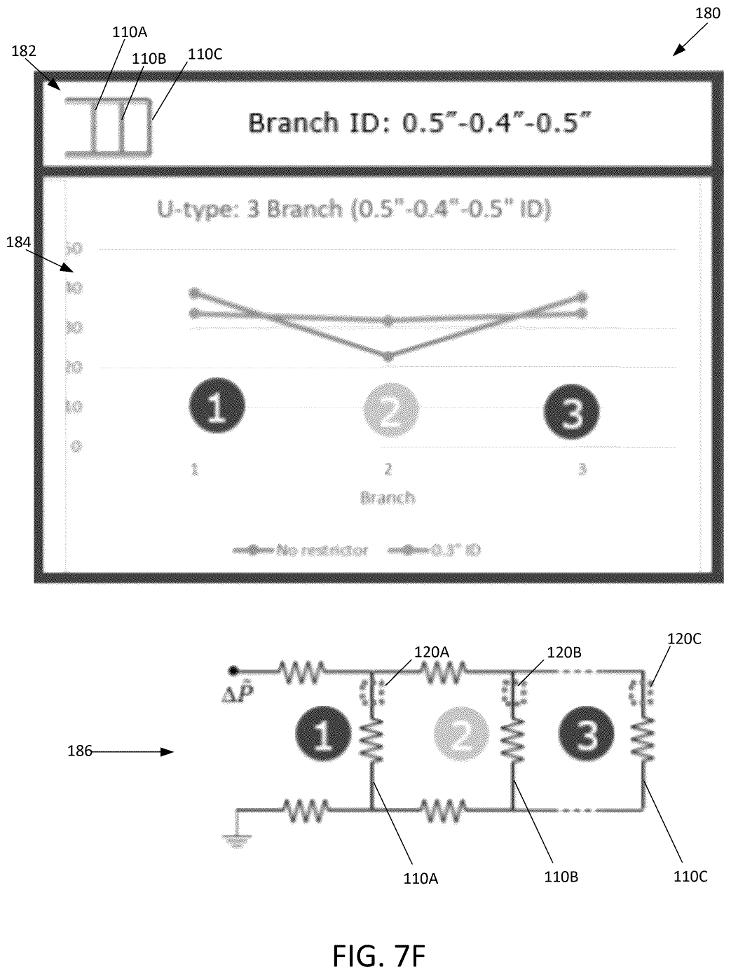

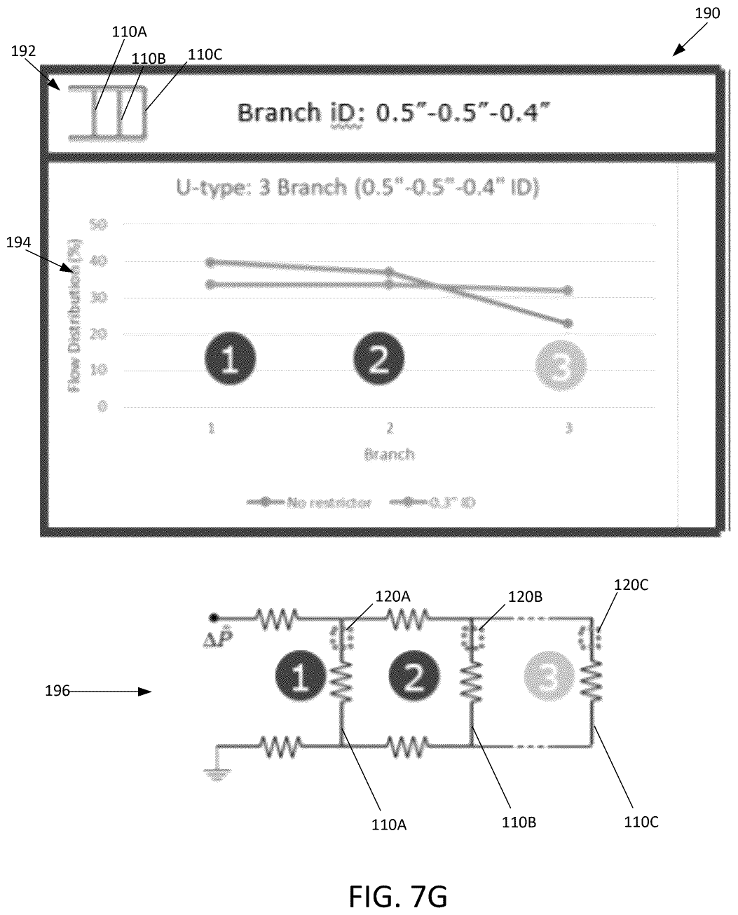

Turning now to FIGS. 7A and 7B, illustrated is a U-type manifold 100 used with flow restrictors 126A-126D (collectively referred to as 126) and pod-type filtration devices 130A-130D (collectively referred to as 130). As illustrated in both FIGS. 7A and 7B, the U-type manifold 100 includes a first conduit 102 and a second conduit 104. The first conduit 102, or main inlet conduit, may include an inlet 106 for the U-type manifold 100, while the second conduit 104, or main outlet conduit, may include an outlet 108 for the U-type manifold 100. The flow stream into the inlet 106 of the first conduit 102 may be in the opposite direction of that of the flow stream out of the outlet 108 of the second conduit 104. The first conduit 102 and the second conduit 104 may be connected to one another via a number of branch conduits 110A-110D (collectively referred to as 110). While FIGS. 7A and 7B illustrate four branch conduits 110, the U-type manifold 100 may include any number of branch conduits 110 greater than one. Each branch conduit 110 may include a first end 112 coupled to the first conduit 102 and an opposite second end 114 coupled to the second conduit 104.

As further illustrated in FIGS. 7A and 7B, each branch conduit 110A-110D of the U-type manifold 100 may include a flow restrictor conduit 120A-120D (collectively referred to as 120). Each flow restrictor conduit 120 may include a first end 122 and an opposite second end 124. The first end 122 of a flow restrictor conduit 120 may be coupled to a branch conduit 110 more proximate to the first end 112 of that branch conduit 110 than the second end 124 of the flow restrictor conduit 120, while the second end 124 of the flow restrictor conduit 120 may be coupled to that same branch conduit 110 more proximate to the second end 114 of that branch conduit 110 than the first end 122 of the flow restrictor conduit 120. For example, the first end 122A of the flow restrictor conduit 120A may be coupled to the branch conduit 110A at a location that is closer to the first end 112A of the branch conduit 110A than the second end 124A of the flow restrictor conduit 120A, while the second end 124A of the flow restrictor conduit 120A may be coupled to the branch conduit 110A at a location that is closer to the second end 114A of the branch conduit 110A than the first end 122A of the flow restrictor conduit 120A. As further illustrated in FIGS. 7A and 7B, each flow restrictor conduit 120 may contain a flow restrictor 126A-126D (collectively referred to as 126) disposed between the first end 122 and the second end 124 of the respective flow restrictor conduit 120.

With continued reference to FIGS. 7A and 7B, disposed along each branch conduit 110A-110D is a pod-type filtration device or pod 130A-130D (collectively referred to as 130), respectively. As illustrated, the filtration device 130 is disposed along its respective branch conduit 110 between the intersection of the second end 124 of the flow restrictor conduit 120 with the branch conduit 110 and the second end 114 of the branch conduit 110. In other words, the filtration device 130 of each branch conduit 110 may be disposed downstream of the respective flow restrictor conduit 120 of each branch conduit 110.

The U-type manifold 100 may further include a flow regulator 140A-140D (collectively referred to as 140), such as, but not limited to, a clamp, a valve, etc. While FIG. 7A schematically illustrates a flow regulator 140 on each branch conduit 110 as a clamp disposed between the intersections of the first and second ends 122, 124 of the flow restrictor conduit 120 with the branch conduit 110, and while FIG. 7B schematically illustrates a flow regulator on each flow restrictor conduit 120 as a clamp disposed between the first and second ends 122, 124 of the flow restrictor conduit 120, the flow regulator 140 may also be a valve (e.g., a stopcock valve) disposed at the intersection of the first end 122 of the flow restrictor conduit 120 with its respective branch conduit 110. Each flow regulator 140 may be configured to direct the flow of fluid through its respective flow restrictor conduit 120 (as schematically shown in FIG. 7A), or may be configured to direct the flow of fluid fully through the respective branch conduit 110 and bypassing the flow restrictor conduit 120 (i.e., the flow regulator 140 may close the path through the flow restrictor conduit 110) (as schematically shown in FIG. 7B). When the manifold is in the first configuration A, as shown in FIG. 7A where the flow regular 140 may be configured to direct the flow of fluid through its respective flow restrictor conduit 120, the U-type manifold is configured for a non-clogging feed stream (e.g., water, buffer, etc.). Conversely, when the manifold is in the second configuration B, as shown in FIG. 7A where the flow regular 140 may be configured to have the flow of fluid bypass the flow restrictor conduit 120, the U-type manifold 100 is configured for a clogging feed stream.

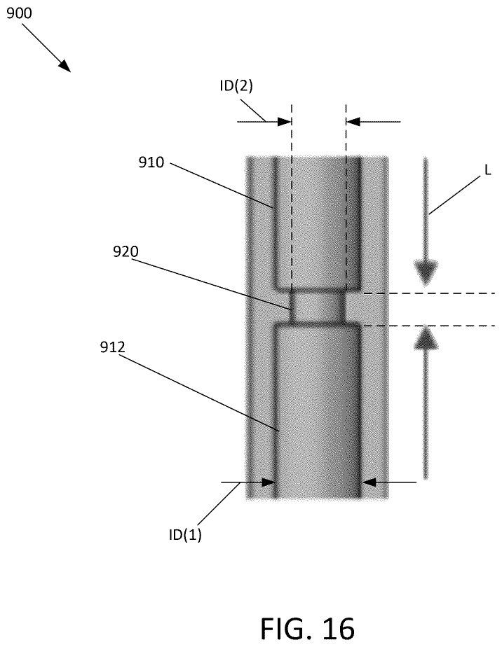

Flow restrictors 126 may be chosen to have a flow resistance greater than (e.g., approximately 3× to 10×) that of the filtration devices 130 (e.g., flow resistance of flow restrictors 126A-126D is three to ten times greater than that of filtration devices 130A-130D, respectively). The resistance of the flow restrictors 126 may be tightly controlled due to the nature of their design and the geometry of the flow restrictor 126 compared with that of the filtration devices 130. If the flow at each branch conduit 110 is only allowed to be directed to the flow restrictor conduit 120 and through a flow restrictor 126 while the branch conduit 110 is clamped or valved (i.e., see FIG. 7A where the U-type manifold 100 is in configuration A), effective flow resistance at each branch conduit 110 can be dominated by the flow restrictor 126, and not by the flow resistance of each filtration device 130. Therefore, with the flow restrictors 126 having the same flow resistance, flow rate into each branch conduit 110 can become uniform or within approximately a 10%-20% variation. As explained above, this is particularly beneficial for non-clogging feed streams. The flow restrictors 126 may include, but are not limited to, a section of the respective conduit having a smaller internal diameter, fittings, pinch clamps, in-line orifice flow restrictors, etc. An example embodiment of the flow restrictor 126 being a smaller internal diameter or a narrowing of the internal diameter of the flow restrictor conduit 120 is shown in FIG. 16.

Because the impact of a clogging feed stream on the flow distribution may not be as significant as that of a non-clogging feed stream, the second configuration B of the U-type manifold 100 may be utilized for clogging feed streams. For clogging feed streams through the U-type manifold 100 in the second configuration B, more flow will initially come into the branch conduits 110 with lower flow resistances (due to the flow resistances of the filtration devices 130) and will bypass the flow restrictor conduits 120 and the respective flow restrictors 126 (because of the flow regulators 140), which will cause the filtration devices 130 in those respective branch conduits 110 to become clogged faster. Then, the clogging feed stream will flow through other filtration devices 130 of the other branch conduits 110 with relatively lower flow resistances at that time. Thus, the overall amount of flow through each filtration device 130 will eventually become balanced.

Turning to FIGS. 7C-7F, illustrated are various charts and schematic depictions of a U-type manifold 100 and the results of a computational analysis on the flow distribution for a variety of scenarios using a U-type manifold 100 as described above in relation to FIGS. 7A and 7B. The computational analysis was performed using SOLIDWORKS® Flow Simulation to determine the effect the presence of a flow restrictor in the U-type manifold 100 would have on the flow distribution through the U-type manifold 100. For the analysis performed in FIGS. 7C-7F, unless otherwise indicated, the U-type manifold 100 may contain first and second conduits 102, 104 having an internal diameter of approximately 0.75 inches, branch conduits 110 having an internal diameter of approximately 0.5 inches, and the branch conduits 110 may be spaced from one another by approximately 5 inches. Moreover, the inlet boundary condition was 300 liters per square meter per hour (hereinafter “LMH”) and the outlet boundary condition was approximately 1 standard atmospheric pressure unit (hereinafter “atm”).

For the first analysis 150 of the U-type manifold 100 as illustrated in FIG. 7C, a U-type manifold 100 was analyzed having ten branch conduits 110A-110J and no filtration devices 130, which is depicted at 152. The diagram at 154 depicts the various pressures throughout the U-type manifold 100 when a feed stream flows through the U-type manifold 100. As shown in the diagram at 154, the branch conduits 110A-110C closest to the inlet 106 and outlet 108 of the U-type manifold 100 experience a lower pressure than that of the branch conduits 110G-110J farthest from the inlet 106 and outlet 108 of the U-type manifold 100. In other words, the farther a branch conduit 110 is from the inlet 106 and outlet 108 of the U-type manifold 100, the greater the pressure experienced by that branch conduit 110. As further depicted in the graph 156, because of the differences in pressures between the branch conduits 110A-110J, there is a large discrepancy between the flow distribution of the first branch conduit 110A and the tenth branch conduit 110J. As shown in the graph 156, when a flow restrictor 120 is not present in the branch conduits 110A-110J of the U-type manifold 100, the first branch conduit 110A receives nearly 25% of the flow distribution while each successive branch conduit 110B-110F receives a smaller amount of the flow distribution then its previous branch conduit 110A-110I. Moreover, the last branch conduit 110J receives less than 5% of the flow distribution of the feed stream flowing through the U-type manifold 100. Thus, if the branch conduits 110A-110J of the U-type manifold 100 depicted at 152 in FIG. 7C did contain filtration devices 130, the filtration device of the first branch conduit 110A would be over-flushed from a non-clogging feed stream, while the filtration device of the last branch conduit 110J would be under-flushed. The schematic diagram 158 in FIG. 7C further depicts the location of the flow restrictors 120A-120J that were utilized to further analyze the flow distribution of the U-type manifold 100. As depicted in the graph 156, the U-type manifold 100 was further analyzed where the branch conduits 110A-110J each contained a flow restrictor 120A-120J. In one iteration, the flow restrictors 120A-120J each had an internal diameter of approximately 0.4 inches, and in a second iteration, the flow restrictors 120A-120J each had an internal diameter of approximately 0.3 inches. As the internal diameters of the flow restrictors 120A-120J were decreased (i.e., the resistance was increased), the flow of the feed stream through the U-type manifold became more evenly distributed. For example, as shown in the graph 156, when the flow restrictors 120A-120J had an internal diameter of approximately 0.3 inches, the first branch conduit 110A received less than 15% of the flow distribution, the last branch conduit 110 received close to 10% of the flow distribution, and the flow distribution of intermediate branch conduits 110B-110I gradually decreased. Finally, as depicted in the table 159 illustrated in FIG. 7C, the pressure differential (AP) with no restrictor was 1.11 psi, where the flow restrictors 120A-120J increase the pressure differential (e.g., 1.72 psi for the flow restrictors 120A-120J having an internal diameter of approximately 0.3 inches).

Turning to FIG. 7D, depicted is the second analysis 160 of the U-type manifold 100 having three branch conduits 110A-110C and no filtration devices 130 as depicted at 162. As indicated on the flow distribution graph 164, each of the branch conduits 110A-110C contained an internal diameter of 0.5 inches. The flow distribution graph 164 depicts the flow distribution of the U-type manifold 100, as depicted at 162, having no flow restrictors, in which each of the second branch conduit 110B and third branch conduit 110C receive a lower percentage of the flow distribution than the first branch conduit 110A. The flow distribution graph 164 also depicts the flow distribution of the U-type manifold 100, as depicted at 166, where each of the branch conduits 110A-110C contains a flow restrictor 120A-120C, respectively, and each of those flow restrictors 120A-120C has an internal diameter of approximately 0.3 inches. Returning to the flow distribution graph 164, the flow distribution of the U-type manifold 100 having flow restrictors 120A-120C, as depicted at 166, is substantially even or equal across each of the branch conduits 110A-110C.

Turning to FIG. 7E, depicted is the third analysis 170 of the U-type manifold 100, as depicted at 172, having three branch conduits 110A-110C and no filtration devices 130, but where the first branch conduit 110A has a different internal diameter than that of the second and third branch conduits 110B, 110C. More specifically, the first branch conduit 110A has an internal diameter of approximately 0.4 inches, while the second and third branch conduits 110B, 110C have internal diameters of approximately 0.5 inches. This causes the first branch conduit 110A to have a greater flow resistance than the second and third branch conduits 110B, 110C. The flow distribution graph 174 depicts the flow distribution of the U-type manifold 100, as depicted at 172, having no flow restrictors, in which each of the second branch conduit 110B and third branch conduit 110C receive a greater percentage of the flow distribution than that of the first branch conduit 110A because of the greater internal diameter of the second and third branch conduits 110B, 110C (i.e., lower resistance of the second and third branches 110B, 110C than that of the first branch 110A). More specifically, the second and third branch conduits 110B, 110C each receive greater than 30% of the flow distribution (with the third branch conduit 110C receiving close to 40% of the flow distribution), while the first branch conduit 110A receives less than 30% of the flow distribution. The flow distribution graph 174 also depicts the flow distribution of the U-type manifold 100, as depicted at 176, where each of the branch conduits 110A-110C contains a flow restrictor 120A-120C, respectively, where each of those flow restrictors 120A-120C has an internal diameter of approximately 0.3 inches. The presence of the flow restrictors 120A-120C effectively causes each of the branches 110A-110C to have the same flow resistance. Returning to the flow distribution graph 174, the flow distribution of the U-type manifold 100 having flow restrictors 120A-120C, as depicted at 176, is substantially even or equal across each of the branch conduits 110A-110C (with the first branch conduit 110A having a slightly lower distribution of the flow, but still greater than 30% of the flow distribution).

Turning to FIG. 7F, depicted is the fourth analysis 180 of the U-type manifold 100, as depicted at 182, having three branch conduits 110A-110C and no filtration devices 130, but where the second branch conduit 110B has a different internal diameter than that of the first and third branch conduits 110A, 110C. More specifically, the second branch conduit 110B has an internal diameter of approximately 0.4 inches, while the first and third branch conduits 110A, 110C have internal diameters of approximately 0.5 inches. This causes the second branch conduit 110B to have a greater flow resistance than the first and third branch conduits 110A, 110C. The flow distribution graph 184 depicts the flow distribution of the U-type manifold 100, as depicted at 182, having no flow restrictors, in which each of the first branch conduit 110A and third branch conduit 110C receive a greater percentage of the flow distribution than that of the second branch conduit 110B because of the greater internal diameter of the first and third branch conduits 110A, 110C (i.e., lower resistance of the first and third branches 110A, 110C than that of the second branch 110B). More specifically, the first and third branch conduits 110A, 110C each receive nearly 40% of the flow distribution, while the first branch conduit 110A receives slightly more than 20% of the flow distribution. The flow distribution graph 184 also depicts the flow distribution of the U-type manifold 100, as depicted at 186, where each of the branch conduits 110A-110C contains a flow restrictor 120A-120C, respectively, where each of those flow restrictors 120A-120C has an internal diameter of approximately 0.3 inches.

The presence of the flow restrictors 120A-120C effectively causes each of the branches 110A-110C to have the same flow resistance. Returning to the flow distribution graph 184, the flow distribution of the U-type manifold 100 having flow restrictors 120A-120C, as depicted at 186, is substantially even or equal across each of the branch conduits 110A-110C (with the second branch conduit 110B having a slightly lower distribution of the flow, but still greater than 30% of the flow distribution).

Turning to FIG. 7G, depicted is the fifth analysis 190 of the U-type manifold 100, as depicted at 192, having three branch conduits 110A-110C and no filtration devices 130, but where the third branch conduit 110C has a different internal diameter than that of the first and second branch conduits 110A, 110B. More specifically, the third branch conduit 110C has an internal diameter of approximately 0.4 inches, while the first and second branch conduits 110A, 110B have internal diameters of approximately 0.5 inches. This causes the third branch conduit 110C to have a greater flow resistance than the first and second branch conduits 110A, 110B. The flow distribution graph 194 depicts the flow distribution of the U-type manifold 100, as depicted at 192, having no flow restrictors, in which each of the first branch conduit 110A and second branch conduit 110B receive a greater percentage of the flow distribution than that of the third branch conduit 110C because of the greater internal diameter of the first and second branch conduits 110A, 110B (i.e., lower resistance of the first and second branches 110A, 110B than that of the third branch 110C). More specifically, the first branch conduit 110A receives nearly 40% of the flow distribution, the second branch conduit 110B receives greater than 35% of the flow distribution, and the third branch conduit 110C receives less than 25% of the flow distribution. The flow distribution graph 194 also depicts the flow distribution of the U-type manifold 100, as depicted at 196, where each of the branch conduits 110A-110C contains a flow restrictor 120A-120C, respectively, where each of those flow restrictors 120A-120C has an internal diameter of approximately 0.3 inches. The presence of the flow restrictors 120A-120C effectively causes each of the branches 110A-110C to have the same flow resistance. Returning to the flow distribution graph 194, the flow distribution of the U-type manifold 100 having flow restrictors 120A-120C, as depicted at 196, is substantially even or equal across each of the branch conduits 110A-110C (with the third branch conduit 110C having a slightly lower distribution of the flow, but still greater than 30% of the flow distribution).

As demonstrated from the above analyses of the U-type manifold 100, including flow restrictors on each of the branch conduits of a U-type manifold 100 causes the flow distribution to be more evenly distributed across the branch conduits despite the number of branch conduits and despite the branch conduits having differing internal diameters.

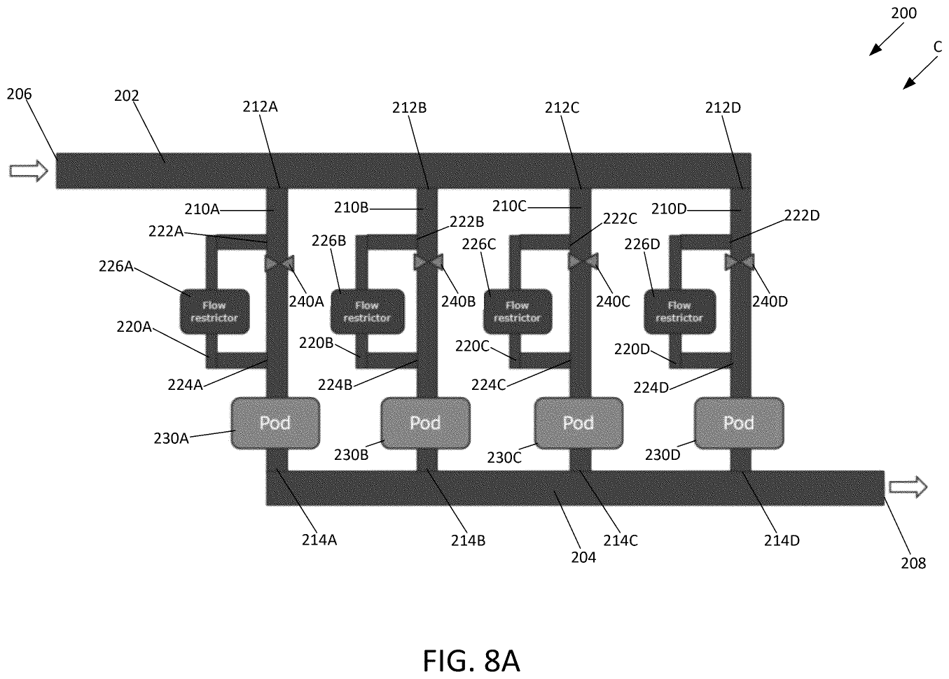

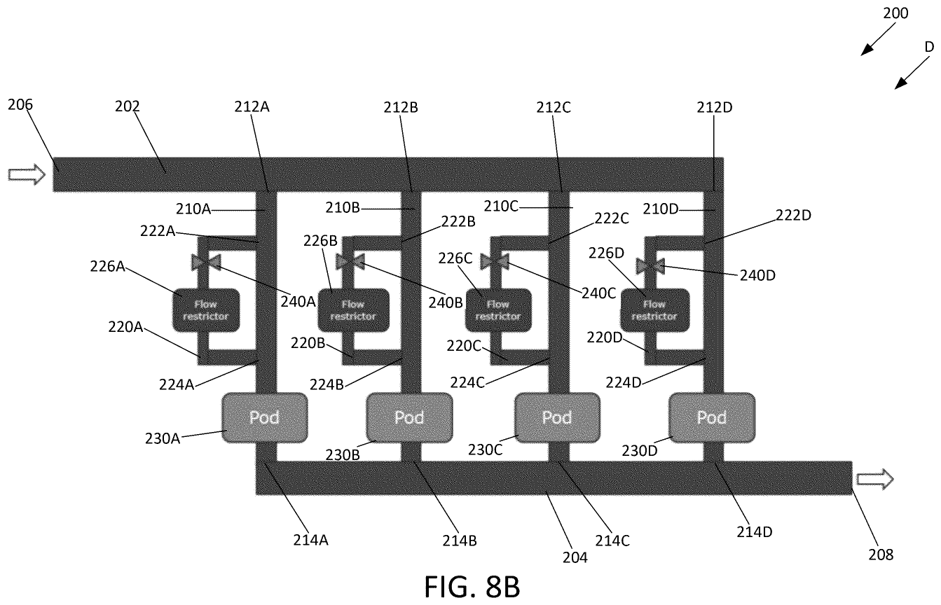

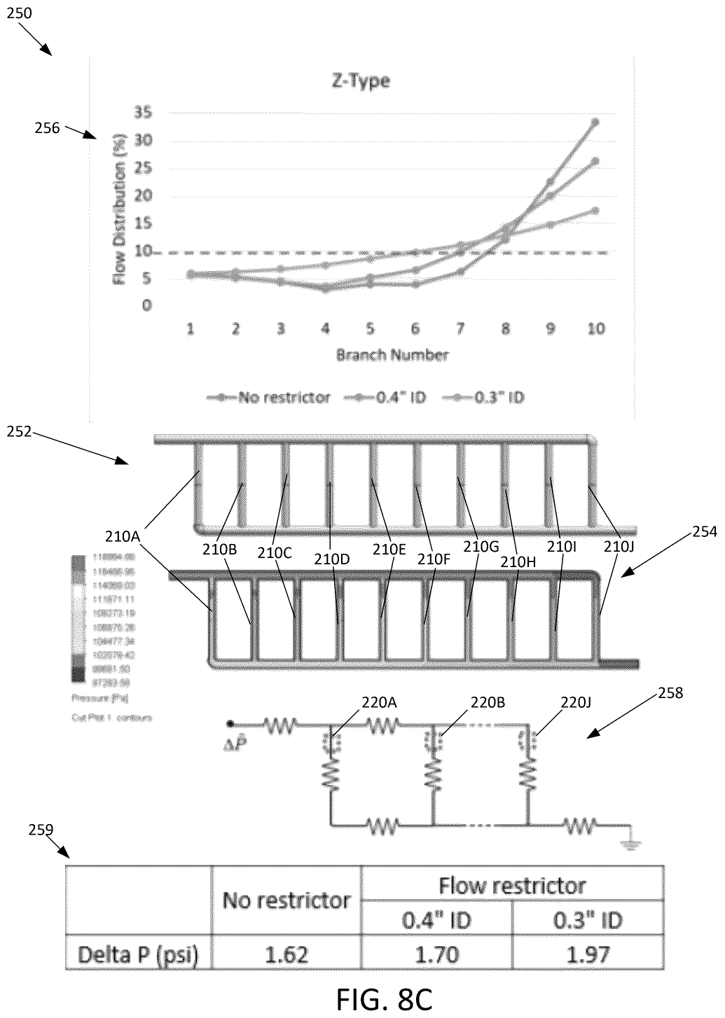

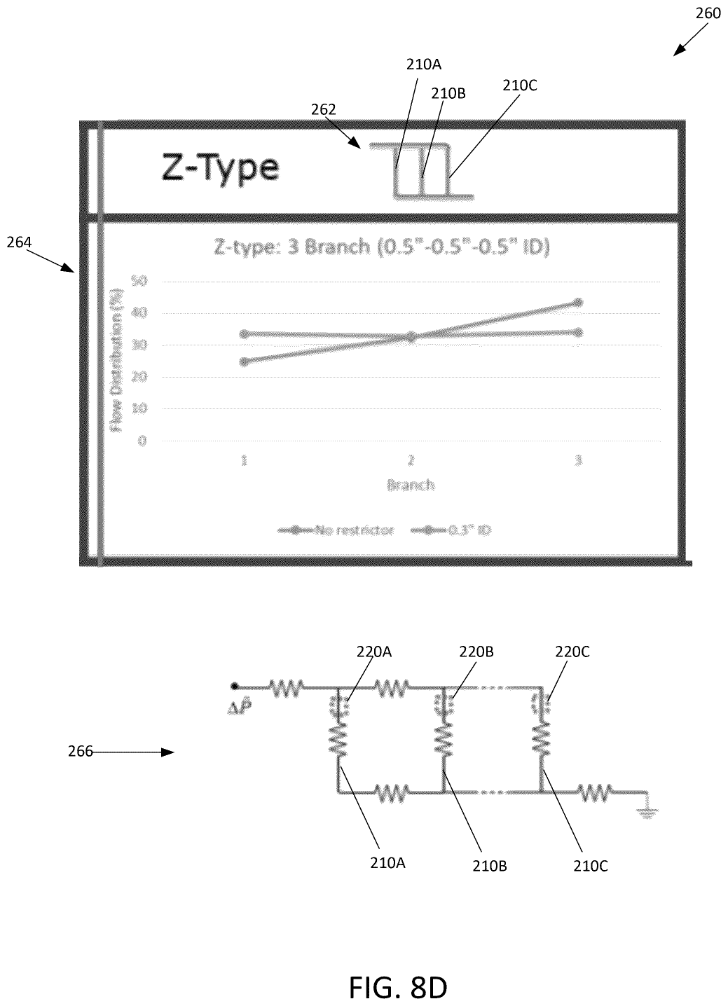

Turning now to FIGS. 8A and 8B, illustrated is a Z-type manifold 200 used with flow restrictors 226A-226D (collectively referred to as 226) and pod-type filtration devices 230A-230D (collectively referred to as 230). As illustrated in both FIGS. 8A and 8B, the Z-type manifold 200 includes a first conduit 202 (also referred to as a main inlet conduit) and a second conduit 204 (also referred to as a main outlet conduit). The first conduit 202 may include an inlet 206 for the Z-type manifold 200, while the second conduit 204 may include an outlet 208 for the Z-type manifold 200. Unlike the U-type manifold 100, the flow stream into the inlet 206 of the first conduit 202 may be in the same direction as that of the flow stream out of the outlet 208 of the second conduit 204. The first conduit 202 and the second conduit 204 may be connected to one another via a number of branch conduits 210A-210D (collectively referred to as 210). While FIGS. 8A and 8B illustrate four branch conduits 210, the Z-type manifold 200 may include any number of branch conduits 210 greater than one. Each branch conduit 210 may include a first end 212 coupled to the first conduit 202 and an opposite second end 214 coupled to the second conduit 204.

As further illustrated in FIGS. 8A and 8B, each branch conduit 210A-210D of the Z-type manifold 200 may include a flow restrictor conduit 220A-220D (collectively referred to as 220). Like the U-type manifold 100, each flow restrictor conduit 220 may include a first end 222 and an opposite second end 224. The first end 222 of a flow restrictor conduit 220 may be coupled to a branch conduit 210 more proximate to the first end 212 of that branch conduit 210 than the second end 224 of the flow restrictor conduit 220, while the second end 224 of the flow restrictor conduit 220 may be coupled to that same branch conduit 210 more proximate to the second end 214 of that branch conduit 210 than the first end 222 of the flow restrictor conduit 220. For example, the first end 222A of the flow restrictor conduit 220A may be coupled to the branch conduit 210A at a location that is closer to the first end 212A of the branch conduit 210A than the second end 224A of the flow restrictor conduit 220A, while the second end 224A of the flow restrictor conduit 220A may be coupled to the branch conduit 210A at a location that is closer to the second end 214A of the branch conduit 210A than the first end 222A of the flow restrictor conduit 220A. As further illustrated in FIGS. 8A and 8B, each flow restrictor conduit 220 may contain a flow restrictor 226A-226D (collectively referred to as 226) disposed between the first end 222 and the second end 224 of the respective flow restrictor conduit 220.

With continued reference to FIGS. 8A and 8B, disposed along each branch conduit 210A-210D is a pod-type filtration device or pod 230A-230D (collectively referred to as 230), respectively. As illustrated, the filtration device 230 is disposed along its respective branch conduit 210 between the intersection of the second end 224 of the flow restrictor conduit 220 with the branch conduit 210 and the second end 214 of the branch conduit 210. In other words, the filtration device 230 of each branch conduit 210 may be disposed downstream of the respective flow restrictor conduit 220 of each branch conduit 210.

Similar to the U-type manifold 100, the Z-type manifold 200 may further include a flow regulator 240A-240D (collectively referred to as 240), such as, but not limited to, a clamp, a valve, etc. While FIG. 8A schematically illustrates a flow regulator 240 on each branch conduit 210 as a clamp disposed between the intersections of the first and second ends 222, 224 of the flow restrictor conduit 220 with the branch conduit 210, and while FIG. 8B schematically illustrates a flow regulator on each flow restrictor conduit 220 as a clamp disposed between the first and second ends 222, 224 of the flow restrictor conduit 220, the flow regulator 240 may also be a valve (e.g., a stopcock valve) disposed at the intersection of the first end 222 of the flow restrictor conduit 220 with its respective branch conduit 210. Each flow regular 240 may be configured to direct the flow of fluid through its respective flow restrictor conduit 220 (as schematically shown in FIG. 8A), or may be configured to direct the flow of fluid through the respective branch conduit 210 and bypassing the flow restrictor conduit 220 (i.e., to close the path through the flow restrictor conduit 210) (as schematically shown in FIG. 8B). When the manifold is in the first configuration C, as shown in FIG. 8A where the flow regular 240 may be configured to direct the flow of fluid through its respective flow restrictor conduit 220, the Z-type manifold 200 is configured for a non-clogging feed stream (e.g., water, buffer, etc.). Conversely, when the Z-type manifold 200 is in the second configuration D, as shown in FIG. 8B where the flow regular 240 may be configured to have the flow of fluid bypass the flow restrictor conduit 220, the Z-type manifold 200 is configured for a clogging feed stream.

Like the flow restrictors 126 of the U-type manifold 100, the flow restrictors 226 of the Z-type manifold 200 may be chosen to have a flow resistance greater than (e.g., approximately 3× to 10×) that of the filtration devices 230 (e.g., flow resistance of flow restrictors 226A-226D is three to ten times greater than that of filtration devices 230A-230D, respectively). The resistance of the flow restrictors 226 may be tightly controlled due to the nature of their design and the geometry of the flow restrictor 226 compared with that of the filtration devices 230. If the flow at each branch conduit 210 is only allowed to be directed to the flow restrictor conduit 220 and through a flow restrictor 226 while the branch conduit 210 is clamped or valved (i.e., see FIG. 8A where the Z-type manifold 200 is in configuration C), the effective flow resistance at each branch conduit 210 can be dominated by the flow restrictor 226, and not by the flow resistance of each filtration device 230. Therefore, with the flow restrictors 226 having the same flow resistance, the flow rate into each branch conduit 210 can become uniform or within approximately a 10%-20% variation across the branch conduits 210. As explained above, this is particularly beneficial for non-clogging feed streams. The flow restrictors 226 may include, but are not limited to, a section of the respective conduit having a smaller internal diameter, fittings, pinch clamps, in-line orifice flow restrictors, etc. An example embodiment of the flow restrictor 226 being a smaller internal diameter or a narrowing of the internal diameter of the flow restrictor conduit 220 is shown in FIG. 16.

Because the impact of a clogging feed stream on the flow distribution may not be as significant as that of a non-clogging feed stream, the second configuration D of the Z-type manifold 200 may be utilized for clogging feed streams. For clogging feed streams through the Z-type manifold 200 in the second configuration D, more flow will initially come into the branch conduits 210 with lower flow resistances (due to the flow resistances of the filtration devices 230) and will bypass the flow restrictor conduits 220 and the respective flow restrictors 226 (because of the flow regulators 240), which will cause the filtration devices 230 in those respective branch conduits 210 to become clogged faster. Then, the clogging feed stream will flow through other filtration devices 230 of the other branch conduits 210 with relatively lower flow resistances at that time. Thus, the overall amount of flow through each filtration device 230 will eventually become balanced.

Turning to FIGS. 8C and 8D, illustrated are various charts and schematic depictions of a Z-type manifold 200 and the results of a computational analysis on the flow distribution for a variety of scenarios using a Z-type manifold 200 as described above in relation to FIGS. 8A and 8B. The computational analysis was performed using SOLIDWORKS® Flow Simulation to determine the effect of the presence of a flow restrictor in the Z-type manifold 200 would have on the flow distribution through the Z-type manifold 200. For the analysis performed in FIGS. 8C and 8D, unless otherwise indicated, the Z-type manifold 200 may contain first and second conduits 202, 204 having an internal diameter of approximately 0.75 inches, branch conduits 210 having an internal diameter of approximately 0.5 inches, and the branch conduits 210 may be spaced from one another by approximately 5 inches. Moreover, the inlet boundary condition was 300 LMH and the outlet boundary condition was approximately 1 atm.

For the first analysis 250 of the Z-type manifold 200 as illustrated in FIG. 8C, a Z-type manifold 200 was analyzed having ten branch conduits 210A-210J and no filtration devices 230, which is depicted at 252. The diagram at 254 depicts the various pressures throughout the Z-type manifold 200 when a feed stream flows through the Z-type manifold 200. As shown in the diagram at 254, the branch conduits 210G-210J closest to the outlet 208 of the Z-type manifold 200 experience a lower pressure than that of the branch conduits 210A-110F closest to the inlet 206 of the Z-type manifold 200. In other words, the closest a branch conduit 210 is to the inlet 206 (and farthest from the outlet 208) of the Z-type manifold 200, the greater the pressure experienced by that branch conduit 210. As further depicted in the graph 256, because of the differences in pressures between the branch conduits 210A-210J, there is a large discrepancy between the flow distribution of the first branch conduit 210A and the tenth branch conduit 210J. As shown in the graph 256, when a flow restrictor 220 is not present in the branch conduits 210A-210J of the Z-type manifold 200, the first branch conduit 210A receives approximately 5% of the flow distribution, the fourth branch conduit 210D receives the lowest flow distribution of less than 5%, and the tenth branch conduit 210J receives the largest flow distribution of nearly 35%. The branch conduits 210A-210D gradually decrease (from the first branch conduit 210A to the fourth branch conduit 210D) in their respective percentage of the flow distribution, while branch conduits 210E-210J gradually increase (from the fifth branch conduit 210E to the tenth branch conduit 210J) in their respective percentage of the flow distribution. In other words, each successive branch conduit 210B-210D receives a smaller amount of the flow distribution then its previous branch conduit, while each successive branch conduit 210E-210J receives a larger amount of the flow distribution than its previous branch conduit. Thus, if the branch conduits 210A-210J of the Z-type manifold 200 depicted at 252 in FIG. 8C did contain filtration devices 230, the filtration devices 230A-230D of the first fourth branch conduits 210A-210D would be under-flushed from a non-clogging feed stream, while the filtration device 230J of the last branch conduit 210J would be over-flushed.

The schematic diagram 258 in FIG. 8C further depicts the location of the flow restrictors 220A-220J that were utilized to further analyze the flow distribution of the Z-type manifold 200. As depicted in the graph 256, the Z-type manifold 200 was further analyzed where the branch conduits 210A-210J each contained a flow restrictor 220A-220J. In one iteration, the flow restrictors 220A-220J each had an internal diameter of approximately 0.4 inches, and in a second iteration, the flow restrictors 220A-220J each had an internal diameter of approximately 0.3 inches. As the internal diameters of the flow restrictors 220A-220J were decreased (i.e., the resistance was increased), the flow of the feed stream through the Z-type manifold 200 became more evenly distributed. For example, as shown in the graph 256, when the flow restrictors 220A-220J had an internal diameter of approximately 0.3 inches, the first branch conduit 210A received slightly more than 5% of the flow distribution, the last branch conduit 210J received slightly more than 15% of the flow distribution, and the flow distribution of intermediate branch conduits 210B-210I gradually increased from the first branch conduit 210A to the last branch conduit 210J. Finally, as depicted in the table 259 illustrated in FIG. 8C, the pressure differential (AP) with no restrictor was 1.62 psi, where the flow restrictors 220A-220J increase the pressure differential (e.g., 1.97 psi for the flow restrictors 220A-220J having an internal diameter of approximately 0.3 inches).

Turning to FIG. 8D, depicted is the second analysis 260 of the Z-type manifold 200 having three branch conduits 210A-210C and no filtration devices 230 as depicted at 262. As indicated on the flow distribution graph 264, each of the branch conduits 210A-210C contained an internal diameter of 0.5 inches. The flow distribution graph 264 depicts the flow distribution of the Z-type manifold 200, as depicted at 262, having no flow restrictors, in which the first branch conduit 210A receives the least amount of the flow distribution and the third branch conduit 210C receives the largest amount of the flow distribution. More specifically, the first branch conduit 210A receives approximately 25% of the flow distribution, the second branch conduit 210B receives slightly more than 30% of the flow distribution, and the third branch conduit 210C receives approximately 45% of the flow distribution. The flow distribution graph 264 also depicts the flow distribution of the Z-type manifold 200, as depicted at 266, where each of the branch conduits 210A-210C contains a flow restrictor 220A-220C, respectively, and each of those flow restrictors 220A-220C has an internal diameter of approximately 0.3 inches. As depicted, the flow distribution of the Z-type manifold 200 having flow restrictors 220A-220C (as shown at 266) is substantially even or equal across each of the branch conduits 210A-210C.

As demonstrated from the above analyses of the Z-type manifold 200, including flow restrictors on each of the branch conduits of a Z-type manifold 200 causes the flow distribution to be more evenly distributed across the branch conduits despite the number of branch conduits.

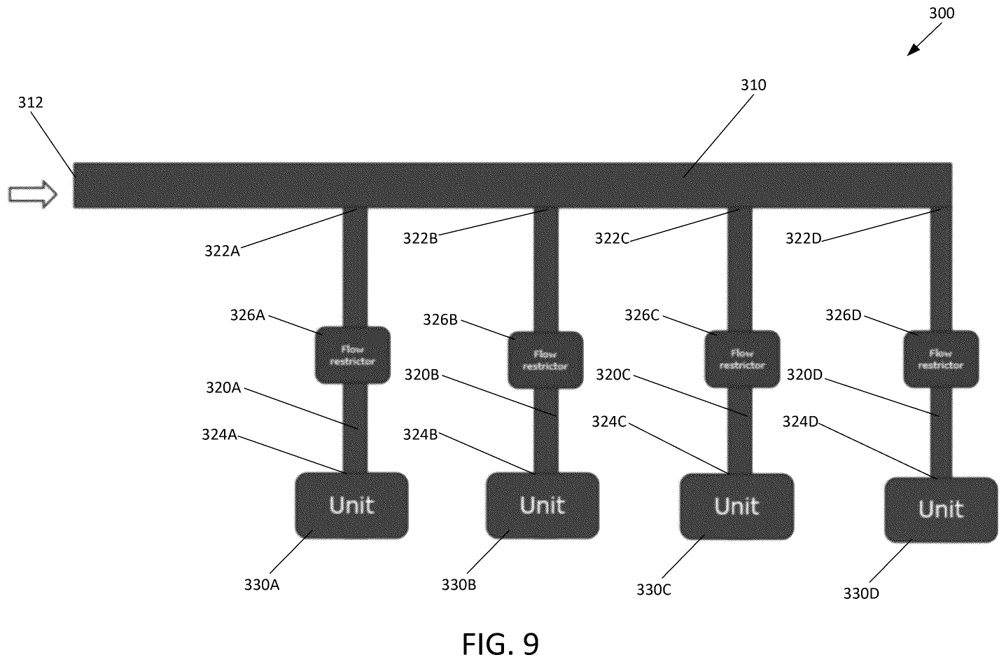

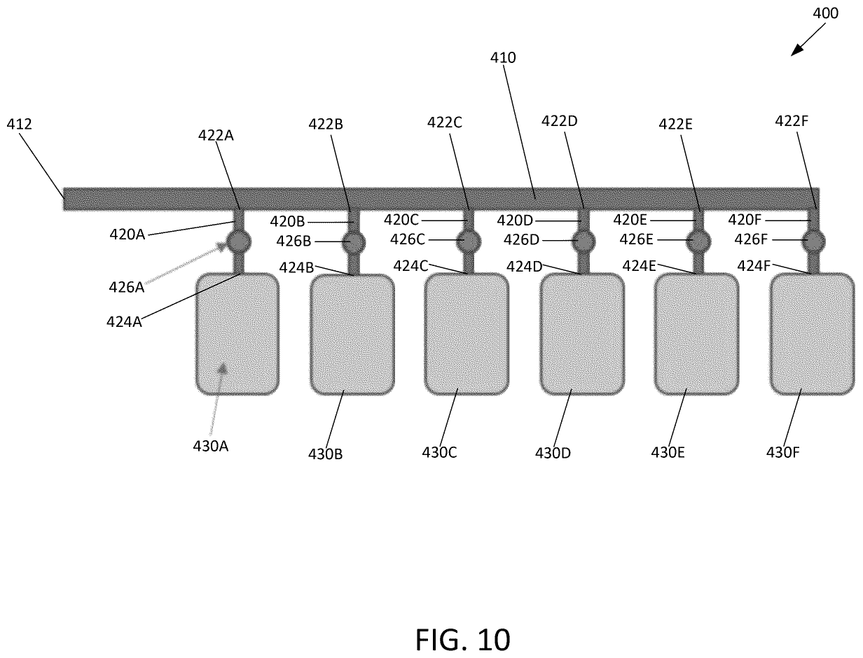

The use of flow restrictors, like that of flow restrictors 126, 226, may also be utilized in manifolds without outlets, like that shown in FIGS. 9, 10, and 12. As illustrated in FIG. 9, the first inlet manifold 300 includes a main conduit 310 and a series of branch conduits 320A-320D (collectively referred to as 320) extending from the main conduit 310. While FIG. 9 depicts the first inlet manifold 300 with four branch conduits 320A-320D, an inlet manifold may include any number of branch conduits 320 greater than one (as illustrated in FIG. 10 with the second inlet manifold 400 that contains six branch conduits 420A-420F and explained in further detail below). As further illustrated in FIG. 9, the main conduit 310 may include an inlet 312 through which a feed stream may flow in order to enter the main conduit 310 and eventually flow into each of the branch conduits 320A-320D. Each of the branch conduits 320A-320D may extend from the same side of the main conduit 310. As illustrated, each branch conduit 320A-320D may include a first end 322A-322D (collectively referred to as 322), respectively, and an opposite second end 324A-324D (collectively referred to as 324), respectively. The first end 322A-322D of each branch conduit 320A-320D may be coupled to the main conduit 310, while the second end 324A-324D of each branch conduit 320A-320D may be coupled to a collection unit 330A-330D (e.g., a container, a tank, a bag, etc.) (collectively referred to as 330) to be filled by the feed stream flowing through the first inlet manifold 300. As FIG. 9 further illustrates, each branch conduit 320A-320D may include a flow restrictor 326A-326D (collectively referred to as 326) that is disposed between the first end 322A-322D and the second end 324A-324D of the respective branch conduit 320A-320D. Like the flow restrictors 126, 226 described above, the flow restrictors 326 of the first inlet manifold 300 may include, but are not limited to, a section of the respective conduit having a smaller internal diameter (see FIG. 16 for an example embodiment), fittings, pinch clamps, in-line orifice flow restrictors, etc. The flow restrictors 326 of the first inlet manifold 300 enable and promote a more uniform distribution of the feed stream into the collection units 330 by increasing the flow resistance of each of the branch conduits 320 such that they are uniform across each of them.

Turning to FIG. 10, illustrated is another example embodiment of a second inlet manifold 400, where the second inlet manifold 410 is configured to supply a series of collection bags 430A-430F with the feed stream (e.g., a biological feed stream) flowing through the second inlet manifold 400. The second inlet manifold 400 may be substantially similar to that of the first inlet manifold 300, such that the second inlet manifold 400 includes a main conduit 410 having an inlet 412 through which a feed stream enters the second inlet manifold 400. The second inlet manifold 400 may also include six branch conduits 420A-420F (collectively referred to as 420), as opposed to the four branch conduits 320A-320D of the first inlet manifold 300, where the branch conduits 420 extend from the same side of the main conduit 410. As further illustrated, each branch conduit 420 includes a flow restrictor 426A-426F (collectively referred to as 426) that is disposed between the first end 422A-422F (collectively referred to as 422) of the respective branch conduit 420 and the second end 424A-424F (collectively referred to as 424) of the respective branch conduit 420. Similar to the first inlet manifold 300, the first ends 422 of the branch conduits 420 of the second inlet manifold 400 may be coupled to the main conduit 410, while the second ends 424 of the branch conduits 420 are coupled to the collection bags 430A-430F (collectively referred to as 430). Like the flow restrictors 126, 226, 326 described above, the flow restrictors 426 of the second inlet manifold 400 may include, but are not limited to, a section of the respective conduit having a smaller internal diameter (see FIG. 16 for an example embodiment), fittings, pinch clamps, in-line orifice flow restrictors, etc. The flow restrictors 426 of the second inlet manifold 400 enable and promote a more uniform distribution of the feed stream into the collection bags 430 by increasing the flow resistance of each of the branch conduits 420 such that they are uniform across each of them.

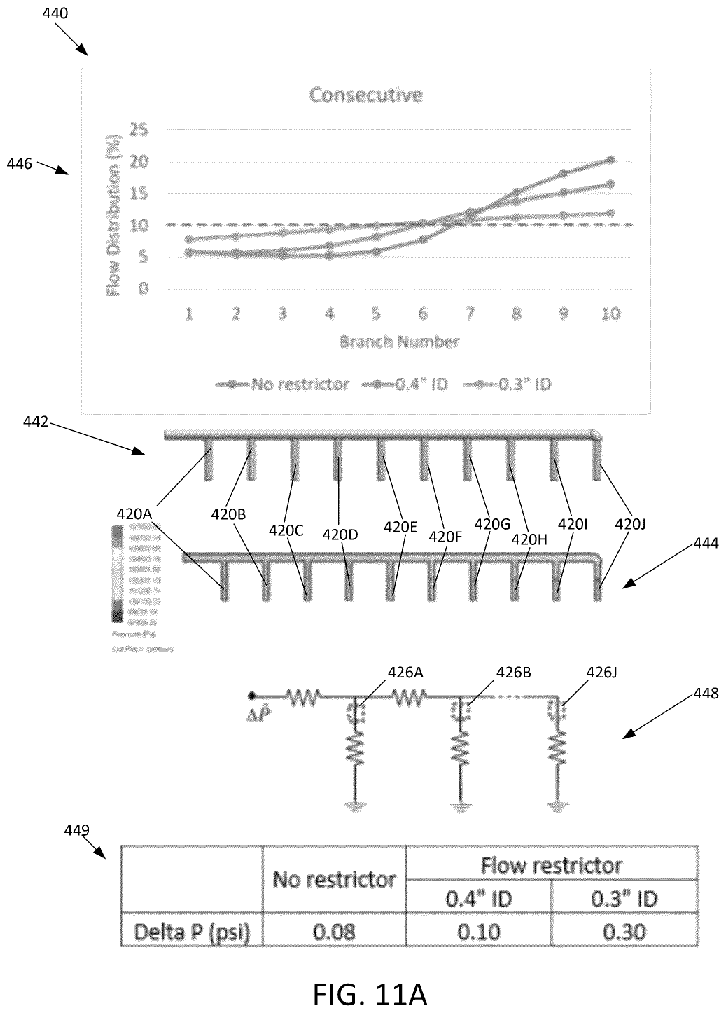

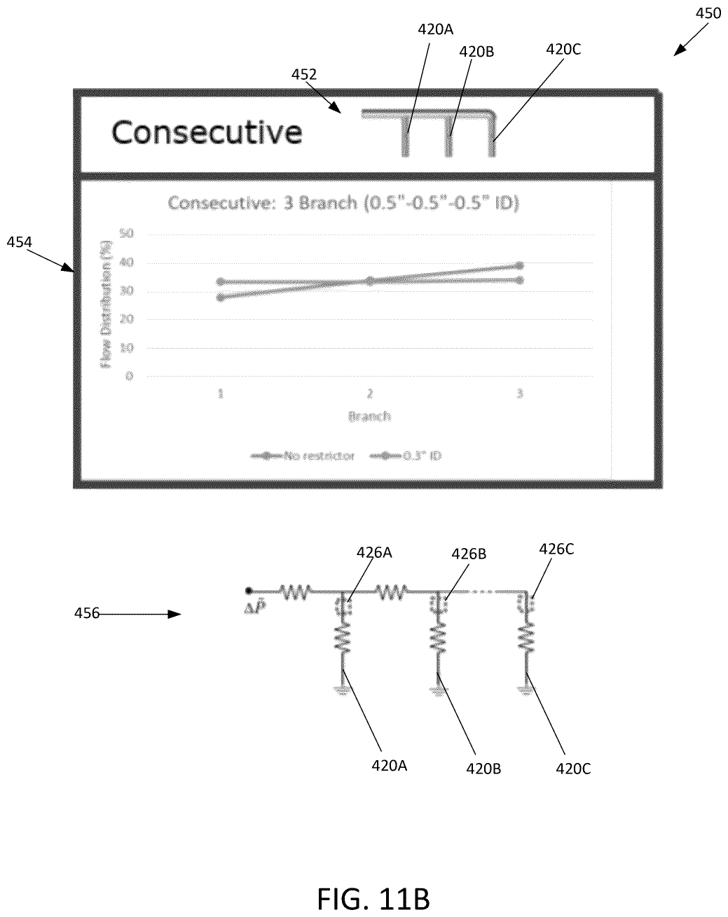

Turning to FIGS. 11A and 11B, illustrated are various charts and schematic depictions of an inlet manifold 400 and the results of a computational analysis on the flow distribution for a variety of scenarios using the inlet type manifold 400 as described above in relation to FIG. 10. The computational analysis was performed using SOLIDWORKS® Flow Simulation to determine the effect of the presence of a flow restrictor in the inlet type manifold 400 would have on the flow distribution through the inlet type manifolds 400. For the analysis performed in FIGS. 11A and 11B, unless otherwise indicated, the inlet type manifold 400 may contain a main conduit 410 having an internal diameter of approximately 0.75 inches, branch conduits 420 having an internal diameter of approximately 0.5 inches, and the branch conduits 420 may be spaced from one another by approximately 5 inches. Moreover, the inlet boundary condition was 300 LMH and the outlet boundary condition was approximately 1 atm.

For the first analysis 440 of the inlet type manifolds 300, 400 as illustrated in FIG. 11A, an inlet type manifold 300, 400 was analyzed having ten branch conduits 420A-420J and no collection bags 430, which is depicted at 442. The diagram at 442 depicts the various pressures throughout the inlet type manifold 400 when a feed stream flows through the inlet type manifold 300, 400. As shown in the diagram at 444, the branch conduits 420F-420J of the inlet type manifold 400 experience a lower pressure than that of the branch conduits 420A-420E closest to the inlet 412 of the inlet type manifold 400. In other words, the closest a branch conduit 420 is to the inlet 412 of the inlet type manifold 400, the greater the pressure experienced by that branch conduit 420. As further depicted in the graph 446, because of the differences in pressures between the branch conduits 420A-420J, there is a large discrepancy between the flow distribution of the first branch conduit 420A and the tenth branch conduit 420J. As shown in the graph 446, when a flow restrictor 426 is not present in the branch conduits 420A-420J of the inlet type manifold 400, the first, second, third, and fourth branch conduits 420A-420D receive approximately 5% of the flow distribution, the fifth through ninth branch conduits 420E-420I gradually increase from slightly more than 5% of the flow distribution (fifth branch conduit 420E) to approximately 18% of the flow distribution (ninth branch conduit 4201), and the tenth branch conduit 420J receives the largest flow distribution of approximately 20%. In other words, the flow distribution is not evenly distributed across the ten branch conduits 420A-420J. Thus, if the branch conduits 420A-420J of the inlet type manifold 400 depicted at 442 in FIG. 11A did contain collection bags 430A-430J, the collection bags 430A-43OF of the first through sixth branch conduits 420A-420F would receive a lower amount of a biological feed stream, while the collection bags 430G-430J of the seventh through tenth branch conduits 420G-420J would receive an overabundance of the biological feed stream. In other words, an inlet type manifold 400 without any flow restrictors is incapable of uniformly distributing a biological feed stream comprising a biopharmaceutical product to the collection bags 430A-430J.

The schematic diagram 448 in FIG. 11A further depicts the location of the flow restrictors 426A-220J that were utilized to further analyze the flow distribution of the inlet type manifold 400. As depicted in the graph 446, the inlet type manifold 400 was further analyzed where the branch conduits 420A-420J each contained a flow restrictor 426A-426J. In one iteration, the flow restrictors 426A-426J each had an internal diameter of approximately 0.4 inches, and in a second iteration, the flow restrictors 426A-426J each had an internal diameter of approximately 0.3 inches. As the internal diameters of the flow restrictors 426A-426J were decreased (i.e., the resistance of the branch conduits 420A-420J was increased), the flow of the feed stream through the inlet type manifold 400 became more evenly distributed. For example, as shown in the graph 446, when the flow restrictors 426A-426J had an internal diameter of approximately 0.3 inches, the first branch conduit 420A received slightly more than 5% of the flow distribution, the last branch conduit 420J received slightly more than 15% of the flow distribution, and the flow distribution of intermediate branch conduits 420B-420I gradually increased from the first branch conduit 210A to the last branch conduit 210J. Finally, as depicted in the table 449 illustrated in FIG. 11A, the pressure differential (AP) with no restrictor was 0.08 psi, where the flow restrictors 426A-426J increase the pressure differential (e.g., 0.30 psi for the flow restrictors 426A-426J having an internal diameter of approximately 0.3 inches).

Turning to FIG. 11B, depicted is the second analysis 450 of the inlet type manifold 400 having three branch conduits 420A-420C and no filtration devices as depicted at 452. As indicated on the flow distribution graph 454, each of the branch conduits 420A-420C contained an internal diameter of 0.5 inches. The flow distribution graph 454 depicts the flow distribution of the inlet type manifold 400, as depicted at 452, having no flow restrictors, in which the first branch conduit 420A receives the least amount of the flow distribution and the third branch conduit 420C receives the largest amount of the flow distribution. More specifically, the first branch conduit 420A receives less than 30% of the flow distribution, the second branch conduit 420B receives approximately 35% of the flow distribution, and the third branch conduit 420C receives slightly less than 40% of the flow distribution. The flow distribution graph 454 also depicts the flow distribution of the inlet type manifold 400, as depicted at 456, where each of the branch conduits 420A-420C contains a flow restrictor 426A-426C, respectively, and each of those flow restrictors 426A-426C has an internal diameter of approximately 0.3 inches. As depicted, the flow distribution of the inlet type manifold 400 having flow restrictors 426A-426C (as shown at 456) is substantially even or equal across each of the branch conduits 420A-420C (each branch conduit 420A-420C receives approximately 33% of the flow distribution).

As demonstrated from the above analyses of the inlet type manifold 400, including flow restrictors on each of the branch conduits of an inlet type manifold 400 causes the flow distribution to be more evenly distributed across the branch conduits despite the number of branch conduits.

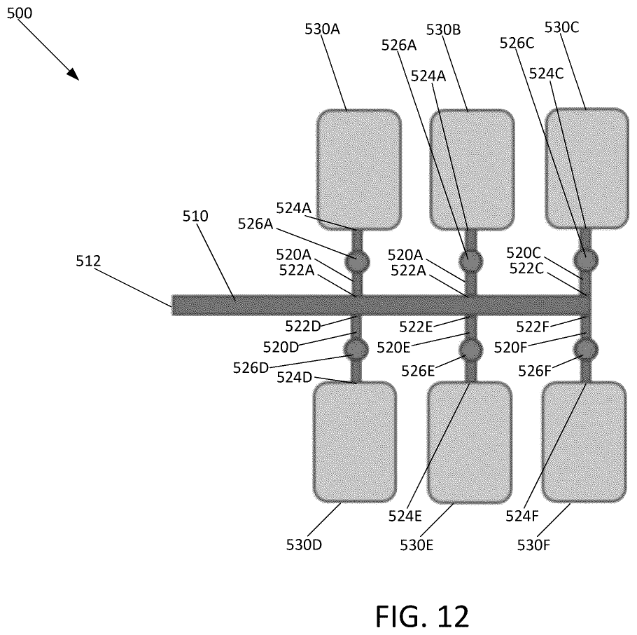

Turning to FIG. 12, illustrated is yet another example embodiment of an inlet manifold 500. The third inlet manifold 500 may be substantially similar to the second inlet manifold 400 in that the third inlet manifold 500 includes a main conduit 510 having an inlet 512 and six branch conduits 520A-520F (collectively referred to as 520) extending from the main conduit 510. In addition, the branch conduits 520 may each include a first end 522A-522F (collectively referred to as 522) coupled to the main conduit 510 and an opposite second end 524A-524F (collectively referred to as 524) coupled to a collection bag 530A-530F (collectively referred to as 530). Disposed between the first and second ends 522, 524 of each branch conduit 520 may be a flow restrictor 526A-526F (collectively referred to as 526) that are configured to promote and enable a more uniform flow of the feed stream (e.g., a biological feed stream) to the collection bags 530. Unlike the first and second inlet manifolds 300, 400, the third inlet manifold 500 illustrated in FIG. 12 depicts the branch conduits 520 extending from the main conduit 510 on different sides (e.g., opposing sides) from one another and are oriented in a symmetrical pattern. In other words, three branch conduits 520A-520C may extend from one side of the main conduit 510, while three branch conduits 520D-520F extend from another side of the main conduit 510. In some embodiments, the branch conduits 520 may be opposed to one another (as depicted in FIG. 12), such that branch conduit 520A is oriented opposite of that of branch conduit 520D, branch conduit 520B is oriented opposite of that of branch conduit 520E, etc. Like that of the other inlet manifolds 300, 400, the flow restrictors 526 of the third inlet manifold 500 enable and promote a more uniform distribution of the feed stream into the collection bags 530. Like the flow restrictors 126, 226, 326, 426 described above, the flow restrictors 526 of the third inlet manifold 500 may include, but are not limited to, a section of the respective conduit having a smaller internal diameter (see FIG. 16 for an example embodiment), fittings, pinch clamps, in-line orifice flow restrictors, etc.

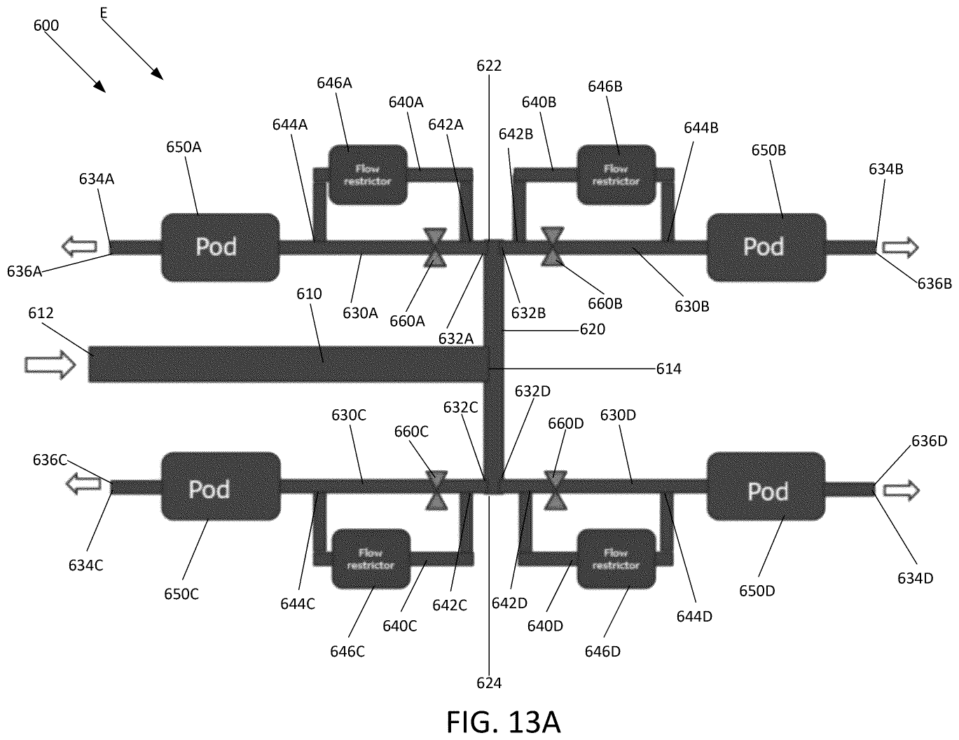

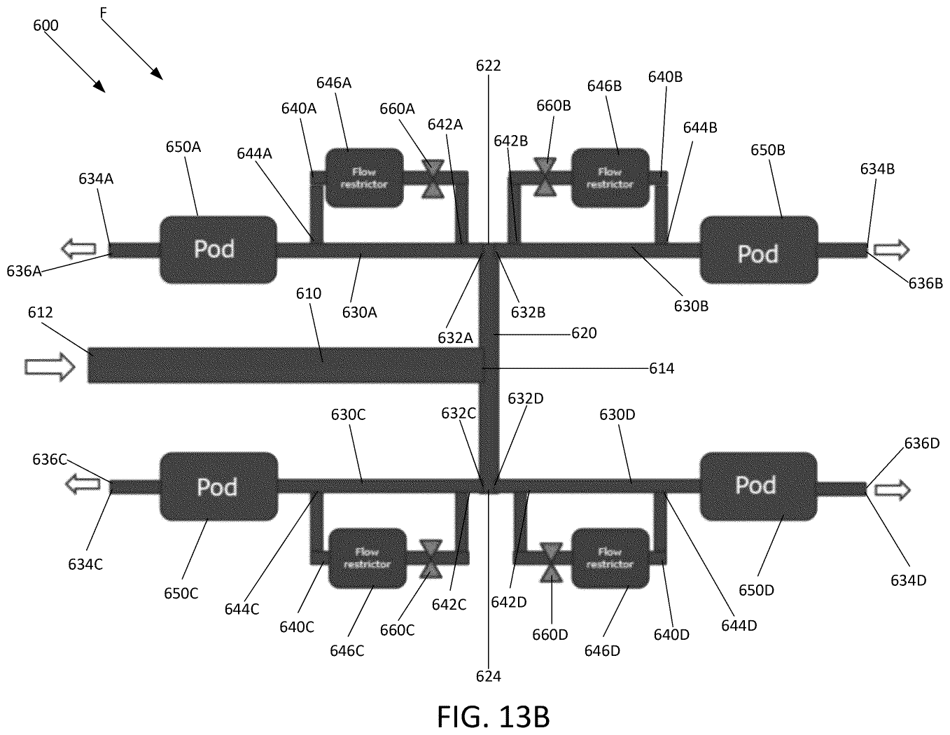

Turning to FIGS. 13A and 13B, illustrated is a bifurcated manifold 600 used with flow restrictors 646A-646D (collectively referred to as 646) and pod-type filtration devices 650A-650D (collectively referred to as 650). As illustrated in both FIGS. 13A and 13B, the bifurcated manifold 600 includes main conduit 610 having a first end 612 and an opposite second end 614, where the first end 612 may serve as an inlet 616 for a feed stream to enter the bifurcated manifold 600. The bifurcated manifold 600 may further include any number of tiered branch conduits to support the symmetrical bifurcation of the feed stream through the bifurcated manifold 600. In the illustrated example embodiment of FIGS. 13A and 13B, the bifurcated manifold 600 includes a primary branch conduit 620 that is coupled to the main conduit 610 at the second end 614, and four secondary branch conduits 630A-630D (collectively referred to as 630) that are each coupled to the primary branch conduit 620. The primary branch conduit 620 may include a first end 622 and an opposite second end 624, where the second end 614 of the main conduit 610 is coupled to the primary branch conduit 620 between the first and second ends 622, 624 of the primary branch conduit 620. As further illustrated in FIGS. 13A and 13B, each of the secondary branch conduits 630 include a first end 632A-632D (collectively referred to as 632) and an opposite second end 634A-634D (collectively referred to as 634), where each of the second ends 634 of the secondary branch conduits 630 serve as outlets 636A-636D (collectively referred to as 636) of the bifurcated manifold 600. The first end 632A of secondary branch conduit 630A and the first end 632B of secondary branch conduit 630B may be coupled to the first end 622 of the primary branch conduit 620, while the first end 632C of secondary branch conduit 630C and the first end 632D of secondary branch conduit 630D may be coupled to the second end 624 of the primary branch conduit 620. Secondary branch conduit 630A and secondary branch conduit 630B may extend from the primary branch conduit 620 in generally opposing directions. Similarly, secondary branch conduit 630C and secondary branch conduit 630D may also extend from the primary branch conduit 620 in generally opposing directions. In other words, secondary branch conduit 630A and secondary branch conduit 630C may extend from the primary branch conduit 620 in the same general direction as one another, while secondary branch conduit 630B and secondary branch conduit 630D may extend from the primary branch conduit 620 in the same general direction. While FIGS. 13A and 13B illustrate one primary branch conduit 620 and four secondary branch conduits 630, the bifurcated manifold 600 may include any number of tiered branch conduits (e.g., primary, secondary, tertiary, etc.), as well as number of conduits for each tier (e.g., any number of primary conduits, any number of secondary conduits, any number of tertiary conduits, etc.).

As further illustrated in FIGS. 13A and 13B, each secondary branch conduit 630A-630D of the bifurcated manifold 600 may include a flow restrictor conduit 640A-640D (collectively referred to as 640). Like the U-type manifold 100 and the Z-type manifold 200, each flow restrictor conduit 640 may include a first end 642A-642D (collectively referred to as 642) and an opposite second end 644A-644D (collectively referred to as 644). The first end 642 of a flow restrictor conduit 640 may be coupled to a respective secondary branch conduit 630 more proximate to the first end 632 of that secondary branch conduit 630 than the second end 644 of the flow restrictor conduit 640, while the second end 644 of the flow restrictor conduit 640 may be coupled to that same secondary branch conduit 630 more proximate to the second end 634 of that secondary branch conduit 630 than the first end 642 of the flow restrictor conduit 640. For example, the first end 642A of the flow restrictor conduit 640A may be coupled to the secondary branch conduit 630A at a location that is closer to the first end 632A of the secondary branch conduit 630A than the second end 644A of the flow restrictor conduit 640A, while the second end 644A of the flow restrictor conduit 640A may be coupled to the secondary branch conduit 630A at a location that is closer to the second end 634A of the secondary branch conduit 630A than the first end 642A of the flow restrictor conduit 640A. As further illustrated in FIGS. 13A and 13B, each flow restrictor conduit 640 may contain a flow restrictor 646A-646D (collectively referred to as 646) disposed between the first end 642 and the second end 644 of the respective flow restrictor conduit 640.

With continued reference to FIGS. 13A and 13B, disposed along each secondary branch conduit 630A-630D is a pod-type filtration device or pod 650A-650D (collectively referred to as 650), respectively. As illustrated, the filtration device 650 is disposed along its respective secondary branch conduit 630 between the intersection of the second end 644 of the flow restrictor conduit 640 with the secondary branch conduit 630 and the second end 634 of the secondary branch conduit 630. In other words, the filtration device 650 of each secondary branch conduit 630 may be disposed downstream of the respective flow restrictor conduit 640 of each secondary branch conduit 630.

Similar to the U-type manifold 100 and the Z-type manifold 200, the bifurcated manifold 600 may further include a flow regulator 660A-660D (collectively referred to as 660), such as, but not limited to, a clamp, a valve, etc. While FIG. 13A schematically illustrates a flow regulator 660 on each secondary branch conduit 630 as a clamp disposed between the intersections of the first and second ends 642, 644 of the flow restrictor conduit 640 with the secondary branch conduit 630, and while FIG. 13B schematically illustrates a flow regulator 660 on each flow restrictor conduit 640 as a clamp disposed between the first and second ends 642, 644 of the flow restrictor conduit 640, the flow regulator 660 may also be a valve (e.g., a stopcock valve) disposed at the intersection of the first end 642 of the flow restrictor conduit 640 with its respective secondary branch conduit 630. In addition, while not illustrated, additional flow regulators may be disposed at the intersection of the secondary branch conduits 630 with the primary branch conduits 620.

Each flow regular 660 may be configured to direct the flow of fluid through its respective flow restrictor conduit 640 (as schematically shown in FIG. 13A), or may be configured to direct the feed stream through the bifurcated manifold 600 to bypass the flow restrictor conduit 640 (i.e., to close the path through the flow restrictor conduit 640) (as schematically shown in FIG. 13B). When the bifurcated manifold 600 is in the first configuration E, as shown in FIG. 13A where the flow regular 660 may be configured to direct the feed stream through its respective flow restrictor conduit 640, the bifurcated manifold 600 is configured for a non-clogging feed stream (e.g., water, buffer, etc.). Conversely, when the bifurcated manifold 600 is in the second configuration F, as shown in FIG. 13B where the flow regular 660 may be configured to have the feed stream bypass the flow restrictor conduit 640, the bifurcated manifold 600 is configured for a clogging feed stream.

Like the flow restrictors 126 of the U-type manifold 100 and the flow restrictors 226 of the Z-type manifold 200, the flow restrictors 646 of the bifurcated manifold 600 may be chosen to have a flow resistance greater than (e.g., approximately 3× to 10×) that of the filtration devices 650 (e.g., flow resistance of flow restrictors 646A-646D is three to ten times greater than that of filtration devices 650A-650D, respectively). The resistance of the flow restrictors 646 may be tightly controlled due to the nature of their design and the geometry of the flow restrictor 646 compared with that of the filtration devices 650. If the flow at each secondary branch conduit 630 is directed toward the flow restrictor conduit 640 and through a flow restrictor 646 while the secondary branch conduit 630 is clamped or valved (i.e., see FIG. 13A where the bifurcated manifold 600 is in the first configuration E), effective flow resistance at each secondary branch conduit 630 can be dominated by the flow restrictors 646, and not by the flow resistance of each filtration device 650. Therefore, with the flow restrictors 646 having the same flow resistance, flow rate into each secondary branch conduit 630 can become uniform or within approximately a 10%-20% variation. As explained above, this is particularly beneficial for non-clogging feed streams. The flow restrictors 646 may include, but are not limited to, a section of the respective conduit having a smaller internal diameter, fittings, pinch clamps, in-line orifice flow restrictors, etc. An example embodiment of the flow restrictors 646 being a smaller internal diameter or a narrowing of the internal diameter of the flow restrictor conduit 640 is shown in FIG. 16.

Because the impact of a clogging feed stream on the flow distribution may not be as significant as that of a non-clogging feed stream, the second configuration F of the bifurcated manifold 600 may be utilized for clogging feed streams. For clogging feed streams through the bifurcated manifold 600 in the second configuration F, more flow will initially come into the secondary branch conduits 630 with lower flow resistances (due to the flow resistances of the filtration devices 650) and will bypass the flow restrictor conduits 640 and the respective flow restrictors 646 (because of the flow regulators 660), which will cause the filtration devices 650 in those respective secondary branch conduits 630 to become clogged faster. Then, the clogging feed stream will flow through the other filtration devices 650 of the other secondary branch conduits 630 with relatively lower flow resistances at that time. Thus, the overall amount of flow through each filtration device 650 will eventually become balanced.

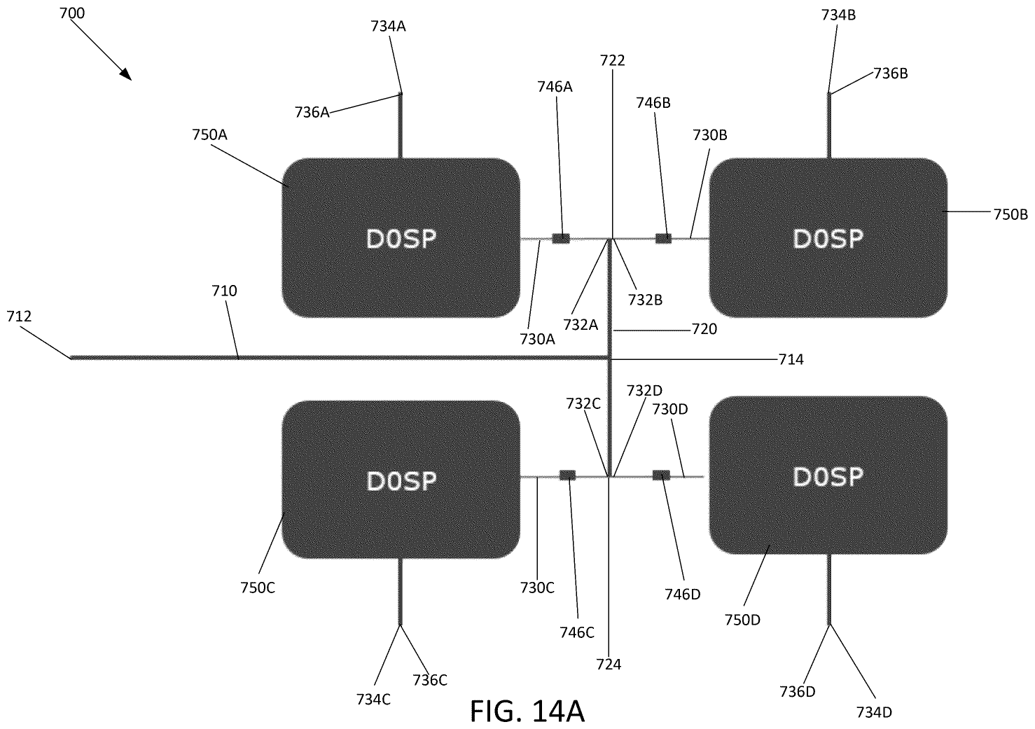

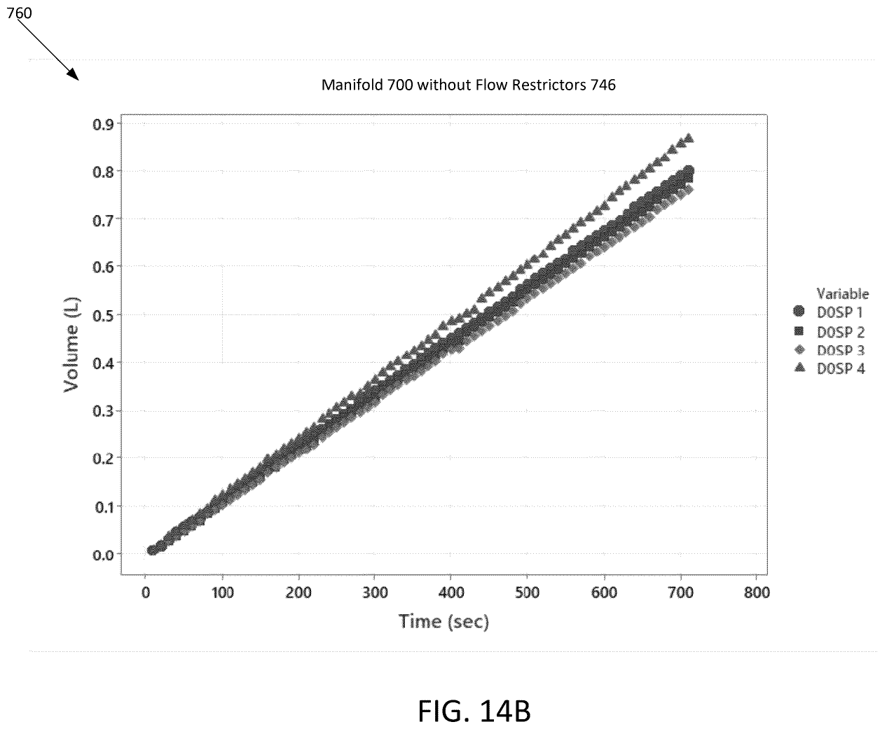

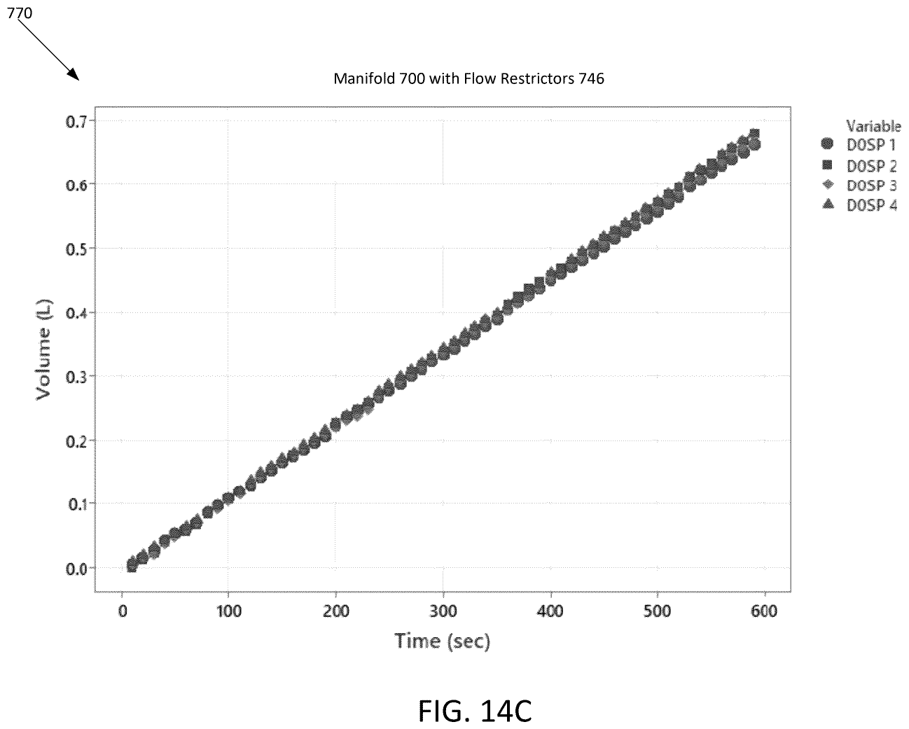

Turning to FIGS. 14A-14C, illustrated is a schematic diagram of a bifurcated manifold 700 equipped with four identical pod filtration devices 750A-750D (collectively referred to as 750), and graphs presenting data of the volume of the flow through the manifold 700 for each pod filtration device 750A-750D receives over a period of time. The schematic bifurcated manifold 700 depicted in FIG. 14A may be a schematic representation of a manifold setup utilized to capture the date presented in the graphs 760, 770 depicted in FIGS. 14B and 14C. Furthermore, the schematic bifurcated manifold 700 depicted in FIG. 14A may be substantially identical to that of the bifurcated manifold illustrated in FIGS. 13A and 13B except that the schematic bifurcated manifold 700 may or may not include flow restrictor conduits like that of the bifurcated manifold 600.

As illustrated in FIG. 14A, the bifurcated manifold 700 includes main conduit 710 having a first end 712 and an opposite second end 714, where the first end 712 may serve as an inlet 716 for a feed stream to enter the bifurcated manifold 700. In the illustrated embodiment, the main conduit 710 may have an internal diameter of approximately 0.25 inches (approximately ¼th of an inch). The bifurcated manifold 700 further includes a primary branch conduit 720 that is coupled to the main conduit 710 at the second end 714, and four secondary branch conduits 730A-730D (collectively referred to as 730) that are each coupled to the primary branch conduit 720. The primary branch conduit 720 may include a first end 722 and an opposite second end 724, where the second end 714 of the main conduit 710 is coupled to the primary branch conduit 720 between the first and second ends 722, 724 of the primary branch conduit 720. The primary branch conduit 720 may have an internal diameter of approximately 0.125 inches (approximately ⅛th of an inch).

As further illustrated in FIG. 14A, each of the secondary branch conduits 730 includes a first end 732A-732D (collectively referred to as 732) and an opposite second end 734A-734D (collectively referred to as 734), where each of the second ends 734 of the secondary branch conduits 730 serve as outlets 736A-736D (collectively referred to as 736) of the bifurcated manifold 700. The first end 732A of secondary branch conduit 730A and the first end 732B of secondary branch conduit 730B may be coupled to the first end 722 of the primary branch conduit 720, while the first end 732C of secondary branch conduit 730C and the first end 732D of secondary branch conduit 730D may be coupled to the second end 724 of the primary branch conduit 720. Secondary branch conduit 730A and secondary branch conduit 730B may extend from the primary branch conduit 720 in generally opposing directions. Similarly, secondary branch conduit 730C and secondary branch conduit 730D may also extend from the primary branch conduit 720 in generally opposing directions. In other words, secondary branch conduit 730A and secondary branch conduit 730C may extend from the primary branch conduit 720 in the same general direction as one another, while secondary branch conduit 730B and secondary branch conduit 730D may extend from the primary branch conduit 720 in the same general direction. Each of the secondary branch conduits 730 may have an internal diameter of approximately 0.0625 inches (approximately 1/16th of an inch).

As further illustrated in FIG. 14A, disposed along each secondary branch conduit 730A-730D is a pod-type filtration device or pod 750A-750D (collectively referred to as 750), respectively. As illustrated, each filtration devices 750 is disposed along its respective secondary branch conduit 730 between the first end 732 and the second end 734 of the respective secondary branch conduit 730. Moreover, each of the pod-type filtration devices 750 may be identical to one another, where each pod-type filtration device 750 may be a depth filter having a non-woven media grade of silica filter aid with polyacrylic fibers. In addition, each of the pod-type filtration devices 750 may have a filter area of approximately 135 cm². Additionally disposed along each secondary branch conduit 730A-730D is a flow restrictor 746A-746D (collectively referred to as 746), where each flow restrictor is disposed between the first end 732 of the secondary branch conduit 730A-730D and the pod-type filtration device 750A-750D. In other words, the filtration device 750 of each secondary branch conduit 730 may be disposed downstream of the respective flow restrictor 746 of each secondary branch conduit 730. The flow restrictors 746 evaluated were substantially identical to one another and had an orifice of approximately 0.02 inches in diameter.

Turning to FIGS. 14B and 14C, illustrated are two graphs 760, 770. The first graph 760 (depicted in FIG. 14B) illustrates a plot of volume vs. time for each of the pod-filtration devices 750 of the manifold 700 during flushing but without any flow restrictors 746. The second graph 770 (depicted in FIG. 14C) illustrates a plot of volume vs. time for each of the pod-filtration devices 750 of the manifold 700 during flushing, but where the manifold 700 includes the flow restrictors 746 as schematically shown in FIG. 14A. As demonstrated between the differences between the first graph 760 and the second graph 770, the presence of the flow restrictors 746 reduced the variability of the flowrate between the pod-type filtration devices 750. More specifically, as shown in the first graph 760 of FIG. 14B, as the time increases, the fourth pod-type filtration device 750D receives a greater amount of volume of the flow than that of the first, second, and third pod-type filtration devices 750A, 750B, 750C. The first pod-type filtration device 750A receives more volume of the flow for a given amount of time than the second and third pod-type filtration devices 750B, 750C, while the third pod-type filtration device 750C receives the lowest volume of the flow for a given amount of time. Conversely, as shown in the second graph 770 of FIG. 14C, the variability in the volume of the flow received by the pod-type filtration devices 750 is reduced (i.e., at a given amount of time, each pod-type filtration device 750 receives a flow that is substantially closer or more equal to that of the other pod-type filtration devices 750 than that shown in the first graph 760 of FIG. 14B where the manifold 700 does not include flow restrictors 746).

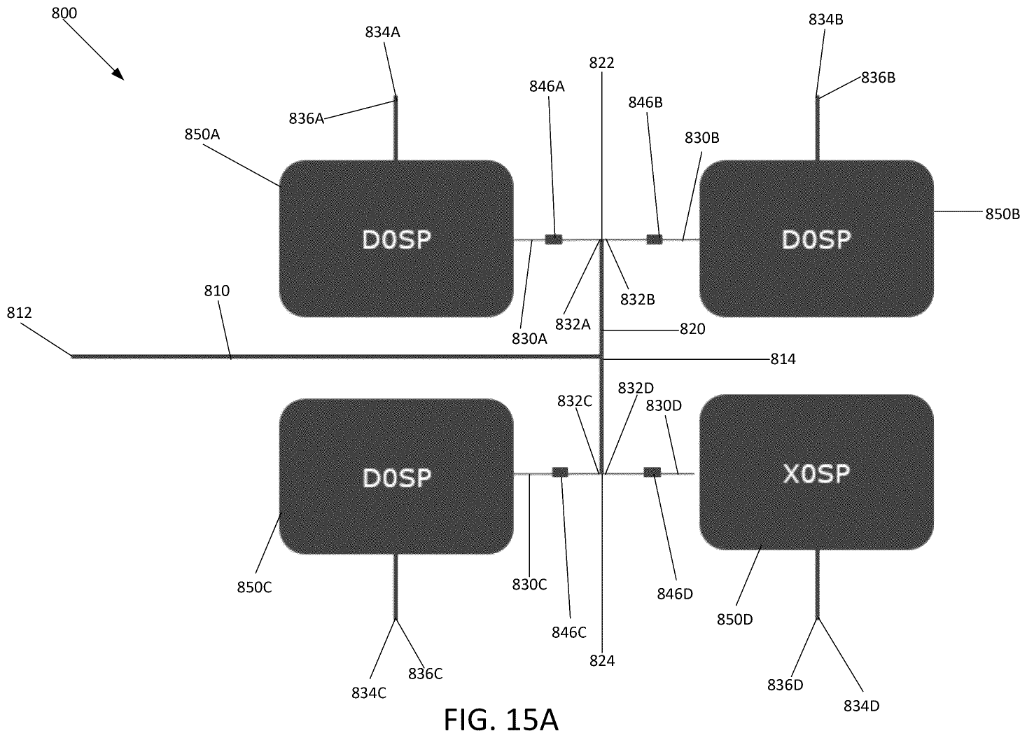

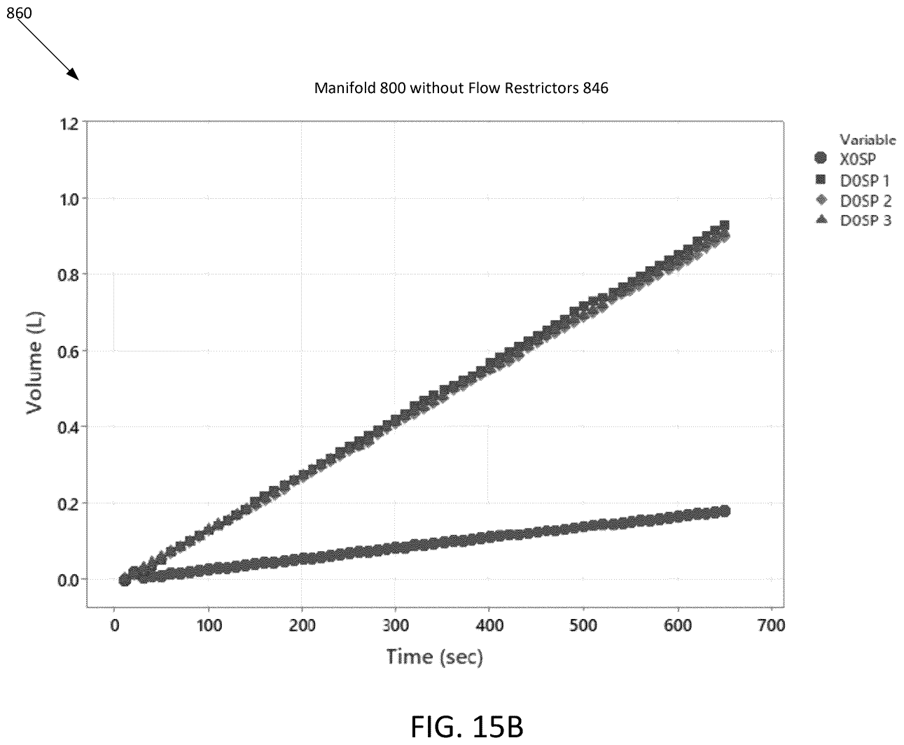

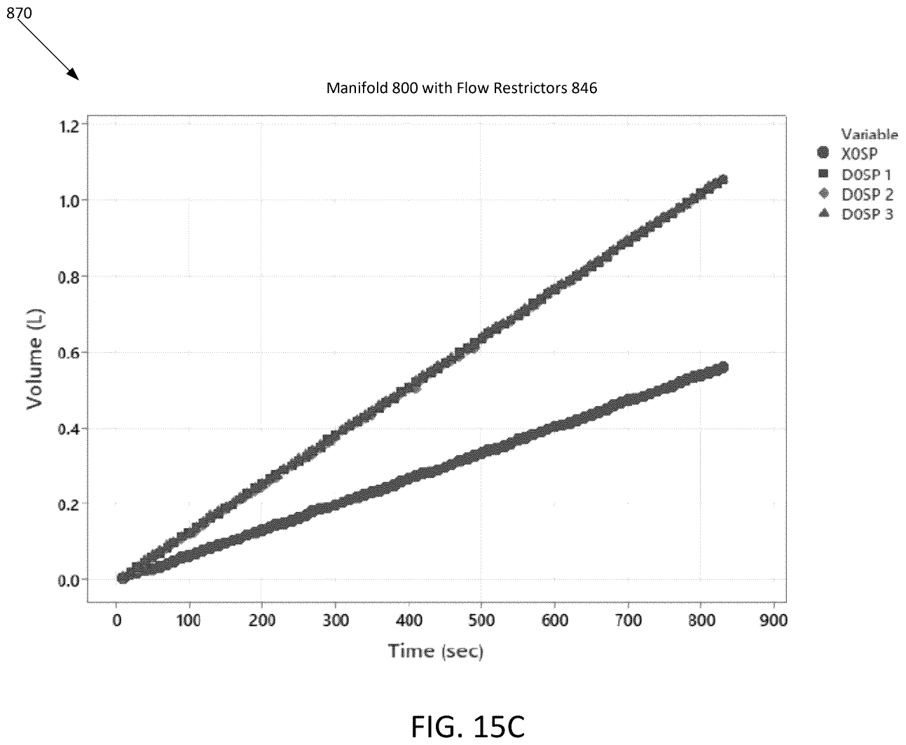

Turning to FIGS. 15A-15C, illustrated is a schematic diagram of a bifurcated manifold 800 equipped with four pod filtration devices 850A-850D (collectively referred to as 850), and graphs 860, 870 presenting data of the volume of the flow through the manifold 800 for each pod filtration device 850A-850D receives over a period of time. The schematic bifurcated manifold 800 depicted in FIG. 15A may be a schematic representation of a manifold setup utilized to capture the date presented in the graphs 860, 870 depicted in FIGS. 15B and 15C. Furthermore, the schematic bifurcated manifold 800 depicted in FIG. 15A may be substantially identical to that of the bifurcated manifold illustrated in FIGS. 13A and 13B except that the schematic bifurcated manifold 800 does not include flow restrictor conduits like that of the bifurcated manifold 600. Thus, the schematic bifurcated manifold 800 may be substantially identical to that of the schematic bifurcated manifold 700 depicted in FIG. 14A other than the filtration devices 850A-850D may differ from one another.

As illustrated in FIG. 15A, the bifurcated manifold 800 includes main conduit 810 having a first end 812 and an opposite second end 814, where the first end 812 may serve as an inlet 816 for a feed stream to enter the bifurcated manifold 800. In the illustrated embodiment, the main conduit 810 may have an internal diameter of approximately 0.25 inches (approximately ¼th of an inch). The bifurcated manifold 800 further includes a primary branch conduit 820 that is coupled to the main conduit 810 at the second end 814, and four secondary branch conduits 830A-830D (collectively referred to as 830) that are each coupled to the primary branch conduit 820. The primary branch conduit 820 may include a first end 822 and an opposite second end 824, where the second end 814 of the main conduit 810 is coupled to the primary branch conduit 820 between the first and second ends 822, 824 of the primary branch conduit 820. The primary branch conduit 820 may have an internal diameter of approximately 0.125 inches (approximately ⅛th of an inch).

As further illustrated in FIG. 15A, each of the secondary branch conduits 830 includes a first end 832A-832D (collectively referred to as 832) and an opposite second end 834A-834D (collectively referred to as 834), where each of the second ends 834 of the secondary branch conduits 830 serve as outlets 836A-836D (collectively referred to as 836) of the bifurcated manifold 800. The first end 832A of secondary branch conduit 830A and the first end 832B of secondary branch conduit 830B may be coupled to the first end 822 of the primary branch conduit 820, while the first end 832C of secondary branch conduit 830C and the first end 832D of secondary branch conduit 830D may be coupled to the second end 824 of the primary branch conduit 820. Secondary branch conduit 830A and secondary branch conduit 830B may extend from the primary branch conduit 820 in generally opposing directions. Similarly, secondary branch conduit 830C and secondary branch conduit 830D may also extend from the primary branch conduit 820 in generally opposing directions. In other words, secondary branch conduit 830A and secondary branch conduit 830C may extend from the primary branch conduit 820 in the same general direction as one another, while secondary branch conduit 830B and secondary branch conduit 830D may extend from the primary branch conduit 820 in the same general direction. Each of the secondary branch conduits 830 may have an internal diameter of approximately 0.0625 inches (approximately 1/16th of an inch).

As further illustrated in FIG. 15A, disposed along each secondary branch conduit 830A-830D is a pod-type filtration device or pod 850A-850D (collectively referred to as 850), respectively. As illustrated, each filtration devices 850 is disposed along its respective secondary branch conduit 830 between the first end 832 and the second end 834 of the respective secondary branch conduit 830. Unlike the pod-type filtration devices 750 of the schematic bifurcated manifold 700, the pod-type filtration devices 850 of the schematic bifurcated manifold 800 are not all identical to one another. More specifically, the first, second, and third pod-type filtration devices 850A, 850B, 850C may be identical to one another, while the fourth pod-type filtration device 850D may be different from the first, second, and third pod-type filtration devices 850A, 850B, 850C. The first, second, and third pod-type filtration devices 850A, 850B, 850C may be depth filters that each have a non-woven layer and a coarse media grade (larger nominal pore size rating) of silica filter aid with polyacrylic fibers. The fourth pod-type filtration device 850D may also be a depth filter, but a depth filter that has a tight grade (smaller nominal pore size rating) of silica filter aid with polyacrylic fibers. Moreover, each of the pod-type filtration devices 850 may have a filter area of approximately 135 cm². The fourth pod-type filtration device 850D may have a substantially tighter permeability than that of the first, second, and third pod-type filtration devices 850A, 850B, 850C.

Additionally disposed along each secondary branch conduit 830A-830D is a flow restrictor 846A-846D (collectively referred to as 846), where each flow restrictor is disposed between the first end 832 of the secondary branch conduit 830A-830D and the pod-type filtration device 850A-850D. In other words, the filtration device 850 of each secondary branch conduit 830 may be disposed downstream of the respective flow restrictor 846 of each secondary branch conduit 830. Like that of the schematic bifurcated manifold 700 of FIG. 14A, the flow restrictors 846 of the schematic bifurcated manifold 800 that were evaluated were substantially identical to one another and had an orifice of approximately 0.02 inches in diameter.

Turning to FIGS. 15B and 15C, illustrated are two graphs 860, 870. The first graph 860 (depicted in FIG. 15B) illustrates a plot of volume vs. time for each of the pod-filtration devices 850 of the manifold 800 during flushing but without any flow restrictors 846. The second graph 870 (depicted in FIG. 15C) illustrates a plot of volume vs. time for each of the pod-filtration devices 850 of the manifold 800 during flushing, but where the manifold 800 includes the flow restrictors 846 as schematically shown in FIG. 15A. As demonstrated between the differences between the first graph 860 and the second graph 870, the presence of the flow restrictors 846 reduced the variability of the flowrate between the pod-type filtration devices 850, and causes the volume of flow to be more equal across the pod-type filtration devices 850, even when the pod-type filtration devices 850 are different from one another and have different resistances. As depicted in both graphs 860, 870 illustrated in FIGS. 15B and 15C, respectively, because the fourth pod-type filtration device 850D has a substantially tighter permeability than that of the first, second, and third pod-type filtration devices 850A, 850B, 850C, the fourth pod-type filtration device 850D receives a significantly smaller amount of the volume of the flow than that of the first, second, and third pod-type filtration devices 850A, 850B, 850C. However, the presence of the flow restrictors 846 in the manifold 800 reduces the difference in the volume of flow between that received by the first, second, and third pod-type filtration devices 850A, 850B, 850C and that received by the fourth pod-type filtration device 850D.

For example, when the manifold 800 is not equipped with flow restrictors 846, the first, second, and third pod-type filtration devices 850A, 850B, 850C, at 600 seconds, each received a flow volume of slightly more than 0.8 L. As further depicted, the fourth pod-type filtration device 850D, at 600 seconds, received a flow volume slightly lower than 0.2 L. Thus, for the manifold 800 without flow restrictors 846, the volume difference between the first, second, and third pod-type filtration devices 850A, 850B, 850C and the fourth pod-type filtration device 850D, at 600 seconds, is greater than 0.6 L.

Conversely, when the manifold 800 is equipped with flow restrictors 846, the first, second, and third pod-type filtration devices 850A, 850B, 850C, at 600 seconds, each received a flow volume of slightly less than 0.8 L, while the fourth pod-type filtration device 850D, at 600 seconds, received a flow volume of approximately 0.4 L. Thus, for the manifold 800 without flow restrictors 846, the volume difference between the first, second, and third pod-type filtration devices 850A, 850B, 850C and the fourth pod-type filtration device 850D, at 600 seconds, is slightly less than 0.4 L.

In addition to reducing the differences of the flow volume between the different types of pod-type filtration devices 850, the presence of the flow restrictors 846 causes the flow volumes of the same pod-type filtration devices 850A, 850B, 850C to converge toward one another. As shown in the first graph 860 of FIG. 15B, as the time increases, the first pod-type filtration device 850D receives a greater amount of volume of the flow than that of the second and third pod-type filtration devices 850B, 850C, while the second pod-type filtration device 850B receives the lowest volume of the flow for the first, second, and third pod-type filtration devices 850A, 850B, 850C. The variability of the flow volume between the first, second, and third pod-type filtration devices 850A, 850B, 850C increases as the time increases. Conversely, as shown in the second graph 870 of FIG. 15C, the variability in the volume of the flow received by the first, second, and third pod-type filtration devices 850A, 850B, 850C is reduced (i.e., at a given amount of time, each of the first, second, and third pod-type filtration devices 850A, 850B, 850C receives a flow that is substantially closer or more equal than that shown in the first graph 860 of FIG. 15B where the manifold 800 does not include flow restrictors 846). Thus, as demonstrated between the first and second graphs 860, 870 of FIGS. 15B and 15C, respectively, the presence of the flow restrictors 846 serves to reduce the variability of the flow volume received by the pod-type filtration devices 850 regardless of whether or not the pod-type filtration devices 850 are identical to one another or are different versions of a pod-type filtration device.

As previously explained, FIG. 16 illustrates an example embodiment of a flow restrictor 920, where the flow restrictor 920 is a narrowing of the internal diameter of the conduit 900 in which the flow restrictor 920 is placed. The flow restrictor 920 illustrated in FIG. 16 may be an example embodiment of the previously described flow restrictors including the flow restrictor 126 of the U-type manifold 100, the flow restrictor 226 of the Z-type manifold 200, the flow restrictors 326, 426, 526 of the inlet manifolds 300, 400, 500, respectively, and the flow restrictors 646, 746, 846 of the bifurcated manifolds 600, 700, 800, respectively. The conduit 900 may have an upstream segment 910 and a downstream segment 912, where the upstream and downstream segments 910, 912 have an internal diameter ID (1). The flow restrictor 920 may be disposed between the upstream and downstream segments 910, 912 of the conduit 900, may have a length L, and may have an internal diameter ID (2). As illustrated, the internal diameter ID (2) of the flow restrictor 920 may be smaller than that of the internal diameter ID (1) of the upstream and downstream segments 910, 912 of the conduit 900. The smaller internal diameter ID (2) of the flow restrictor 920 may be configured to impart a flow resistance to a feed stream flowing through the conduit 900 by constricting the area through which the feed stream must flow for at least a partial length of the conduit 900 (i.e., the length L of the flow restrictor 920).

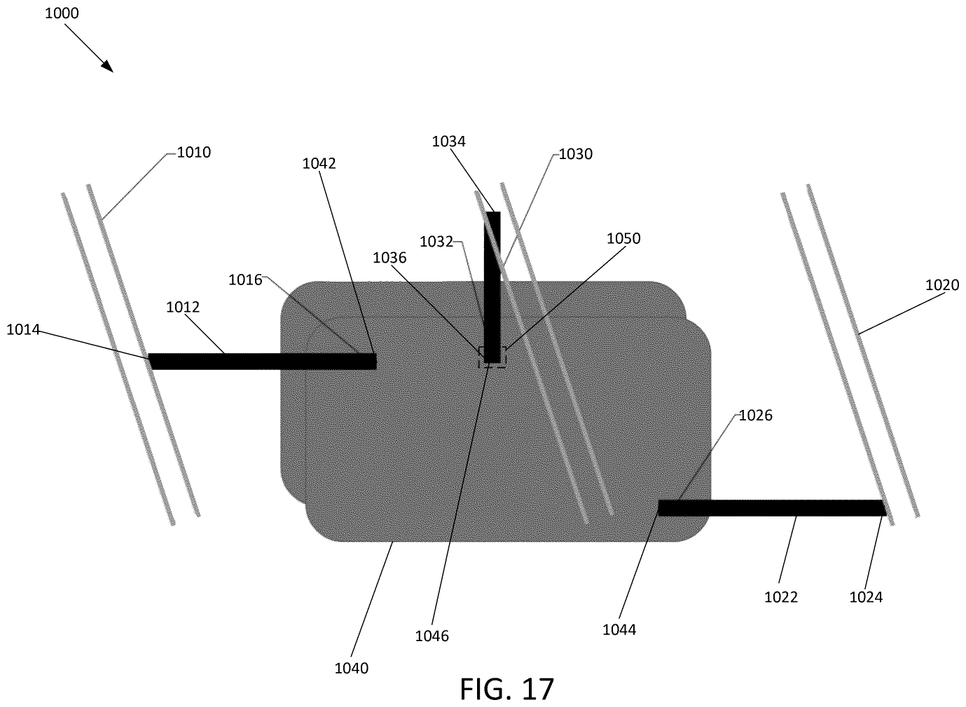

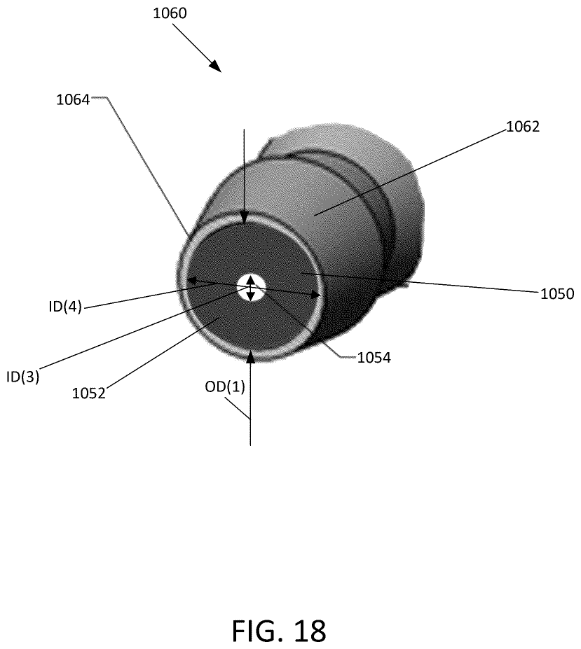

Turning to FIGS. 17 and 18, illustrated is another embodiment for integrating flow restrictors into filtration device manifolds (e.g., U-type, Z-type, and bifurcated manifolds). FIG. 17 illustrates a schematic diagram of a section of a flow manifold 1000, where the depicted section may include an inlet conduit 1010, an outlet conduit 1020, a vent conduit 1030, and a filtration device 1040 coupled to the inlet conduit 1010, the outlet conduit 1020, and the vent conduit 1030. While FIG. 17 only illustrates a single filtration device 1040, the flow manifold 1000 may be configured to be connected to a plurality of filtration devices 1040. The inlet conduit 1010 may further include an inlet connection 1012 that serves as the connection between the inlet conduit 1010 and the filtration device 1040. In the illustrated embodiment, the inlet connection 1012 may include a first end 1014 coupled to the inlet conduit 1010 and an opposite second end 1016 coupled to the filtration device 1040. Similarly, the outlet conduit 1020 may further include an outlet connection 1022 that serves as the connection between the outlet conduit 1020 and the filtration device 1040. In the illustrated embodiment, the outlet connection 1022 may include a first end 1024 coupled to the outlet conduit 1020 and an opposite second end 1026 coupled to the filtration device 1040. As further illustrated, the vent conduit 1030 may further include a vent connection 1032 that serves as the connection between the vent conduit 1030 and the filtration device 1040. In the illustrated embodiment, the vent connection 1032 may include a first end 1034 coupled to the vent conduit 1030 and an opposite second end 1036 coupled to the filtration device 1040. The inlet, outlet and vent conduits 1010, 1020, 1030 may be constructed to have an internal diameter large enough to transport the fluid flow of all of the filtration devices 1040 (not shown for illustrative purposes) that are connected to the flow manifold 1000 with minimal pressure drop. The inlet, outlet, and vent connections 1012, 1022, 1032 may be constructed to have an internal diameter that is smaller in size than the internal diameter of the inlet, outlet, and vent conduits 1010, 1020, 1030 but still large enough such that the inlet, outlet and vent connections 1012, 1022, 1032 are suitable for transporting the fluid flow of a single filtration device 1040 with minimal pressure drop. Moreover, the connection of the conduits 1010, 1020, 1030, the connection lines 1012, 1022, 1032, and the filtration devices 1040 may be made aseptically.

As depicted in FIG. 17, the filtration device 1040 may include a device inlet 1042, a device outlet 1044, and a vent port 1046. The second end 1016 of the inlet connection 1012 may be coupled to the device inlet 1042 of the filtration device 1040, while the second end 1026 of the outlet connection 1022 may be coupled to the device outlet 1044 of the filtration device 1040. Furthermore, the second end 1036 of the vent connection 1032 may be coupled to the vent port 1046 of the filtration device 1040. In a typical or conventional setup of the filtration device 1040, a feed stream may flow into the filtration device 1040 via the device inlet 1042, flow through the filtration device 1040 in order to complete a filtering or flushing process, and then flow out of the filtration device 1044 via the device outlet 1044 while the filtration device 1040 is vented via the vent port 1046 (i.e., facilitates air removal from the filtration device 1040). However, in the depicted example embodiment, a flow restrictor 1050 may be retrofitted or implemented in either the vent port 1046, proximate to the vent port 1046, or in the second end 1036 of the vent connection 1032 that is configured to impart a flow resistance onto a flow of liquid. In some embodiments, the vent port 1046 of the filtration device 1040 may be equipped with a hose barb 1060 like that illustrated in FIG. 18, where the flow restrictor 1050 may be disposed (e.g., retrofitted) within an end of the hose barb 1060. As illustrated in FIG. 18, the flow restrictor 1050 may include a body 1052 having an outer diameter OD (1) and a restrictor opening 1054, which has an internal diameter ID (3). The hose barb 1060 may include an outer surface 1062 and an internal conduit 1064 having an internal diameter ID (4). The outer diameter OD (1) of the body 1052 of the flow restrictor 1050 may be approximately equal to the internal diameter ID (4) of the internal conduit 1064 of the hose barb 1060 such that the flow restrictor 1050 may be configured to be disposed within the internal conduit 1064 of the hose barb 1060 (e.g., via a friction or interference fit). As further illustrated, the internal diameter ID (3) of the restrictor opening 1054 of the flow restrictor 1050 may be smaller than the internal diameter ID (4) of the internal conduit 1064 of the hose barb 1060. While not illustrated, the internal diameter ID (3) of the restrictor opening 1054 of the flow restrictor 1050 may also be smaller in size than the internal diameters of the inlet, outlet, and vent conduits 1010, 1020, 1030, as well as the internal diameters of the inlet, outlet, and vent connections 1012, 1022, 1032.

As previously explained, in a typical or conventional setup of the filtration device 1040, a feed stream (both a non-clogging feed stream and a clogging feed stream) may flow into the filtration device 1040 via the device inlet 1042, flow through the filtration device 1040 in order to complete a filtering process, and then flow out of the filtration device 1044 via the device outlet 1044 while the filtration device 1040 is vented via the vent port 1046 (i.e., facilitates air removal from the filtration device 1040). However, with the flow restrictor placed within the vent port 1046 of the filtration device, a non-clogging feed stream (or non-fouling flow such as a wetting flow) could be directed through the vent conduit 1030, the vent connection 1032, and into the filtration device 1040 via the flow restrictor 1050 and the vent port 1046. Venting to remove air from the upstream of the filtration device 1040 could be run in either direction using the vent conduit 1030 and the inlet conduit 1010. Then, the non-clogging feed stream (e.g., wetting flow) could flow in a forward direction with the vent conduit 1030 and vent connection 1032 serving as the inlet to the filtration device 1040 for the non-clogging feed stream and the outlet conduit 1020 and outlet connection 1022 still serving as the outlet of the filtration device 1040 for the non-clogging feed stream. In another embodiment, the non-clogging feed stream could flow in a reverse direction with the outlet conduit 1020 and outlet connection 1022 serving as the inlet of the filtration device 1040 for the non-clogging feed stream and the vent conduit 1030 and vent connection 1032 serving as the outlet of the filtration device 1040 for the non-clogging feed stream. The flow direction of the non-clogging feed stream may be chosen to best integrate with the sources of the non-clogging feed stream and end receiving vessels.

The smaller internal diameter ID (3) of the restrictor opening 1054 of the flow restrictor 1050 may be configured to impart a flow resistance to a non-clogging feed stream flowing through the flow manifold 1000 by constricting the area through which the non-clogging feed stream must flow. The internal diameter ID (3) of the restrictor opening 1054 of the flow restrictor 1050 may be chosen to select the pressure drop or resistance desired at the anticipated feed stream flow rate or pressures desired for wetting the filtration device 1040. Moreover, incorporating the flow restrictor 1050 into the vent port 1046 of the filtration device 1040 enables a more uniform distribution of the feed stream, especially a non-clogging feed stream, with existing flow manifolds 1000 (i.e., without manipulating the existing manifolds 1000 and/or with minimal changes or additional parts) by imparting a higher flow resistance to the vent pathway (i.e., the pathway through the vent conduit 1030, vent connection 1032, hose barb 1060 equipped with the flow restrictor 1050, and vent port 1046). The incorporation of the flow restrictor 1050 as described above also eliminates the need for the creation of manifolds 100, 200, 600 containing separate flow restrictor conduits 120, 220, 640, respectively.

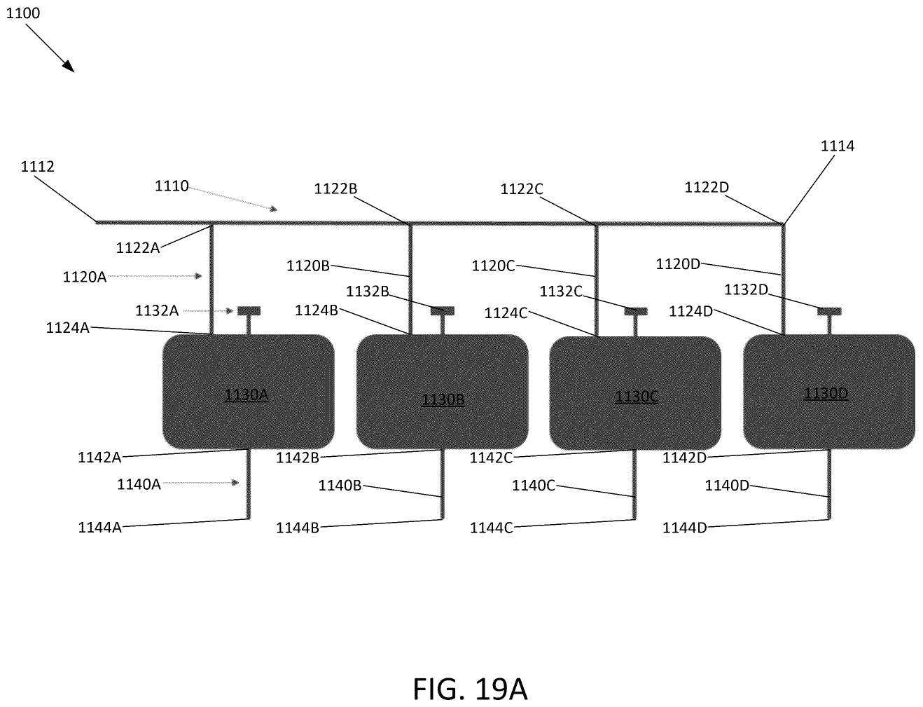

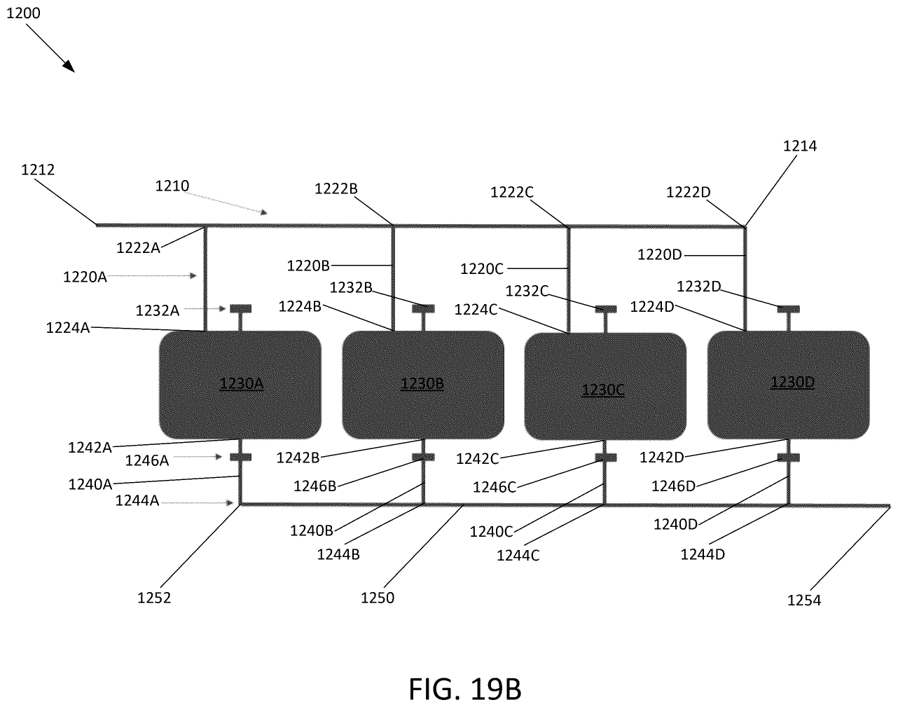

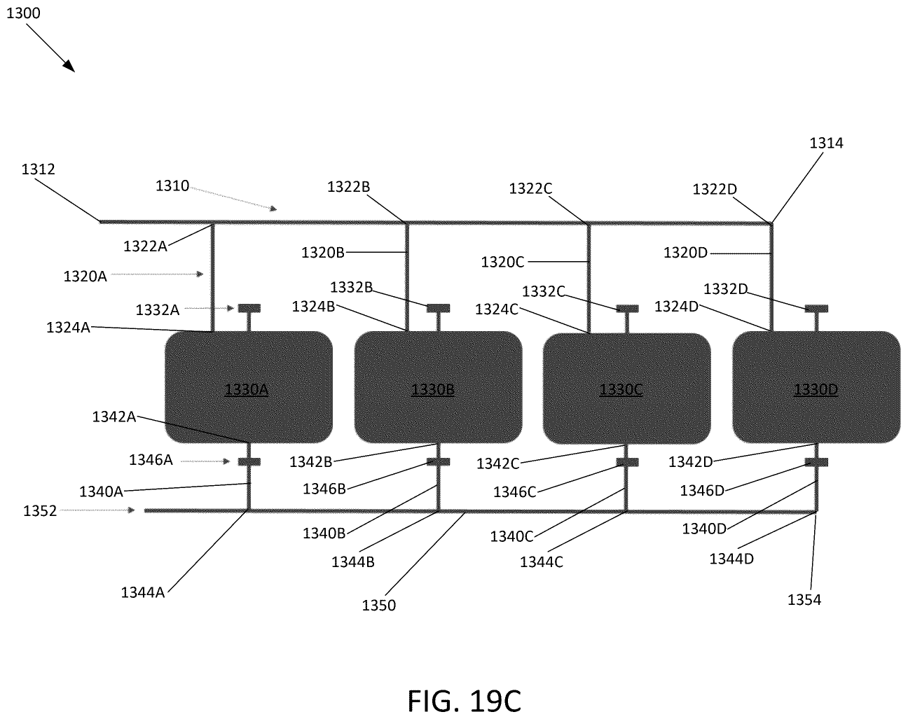

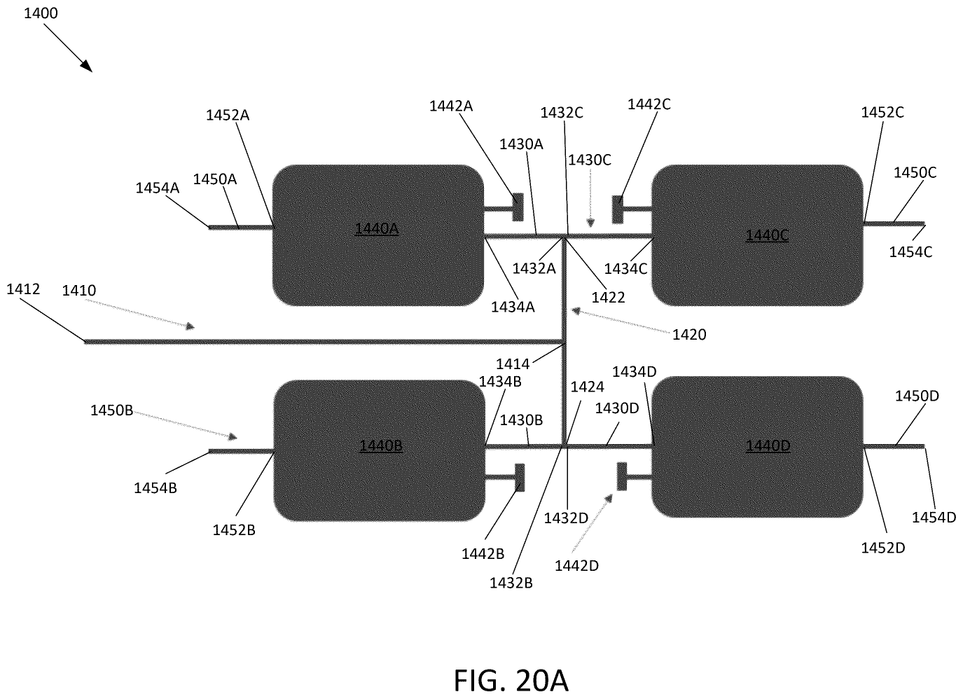

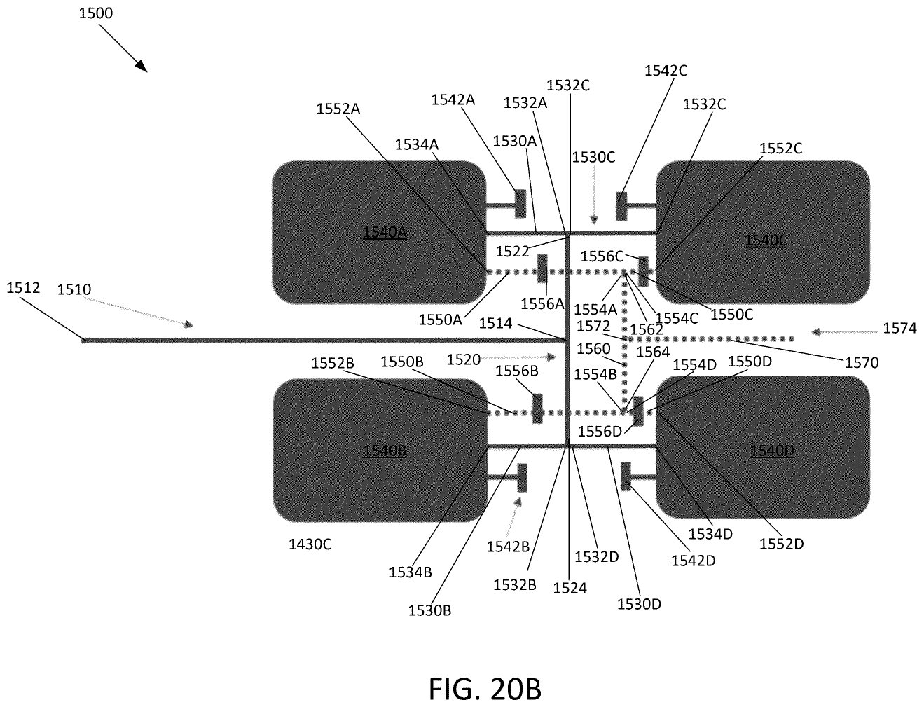

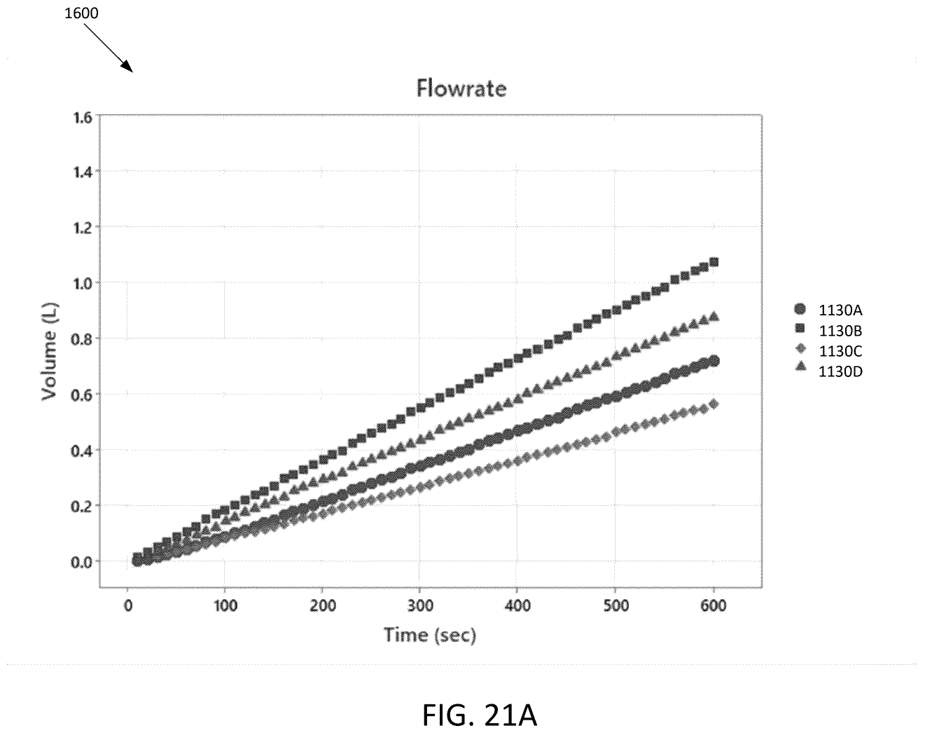

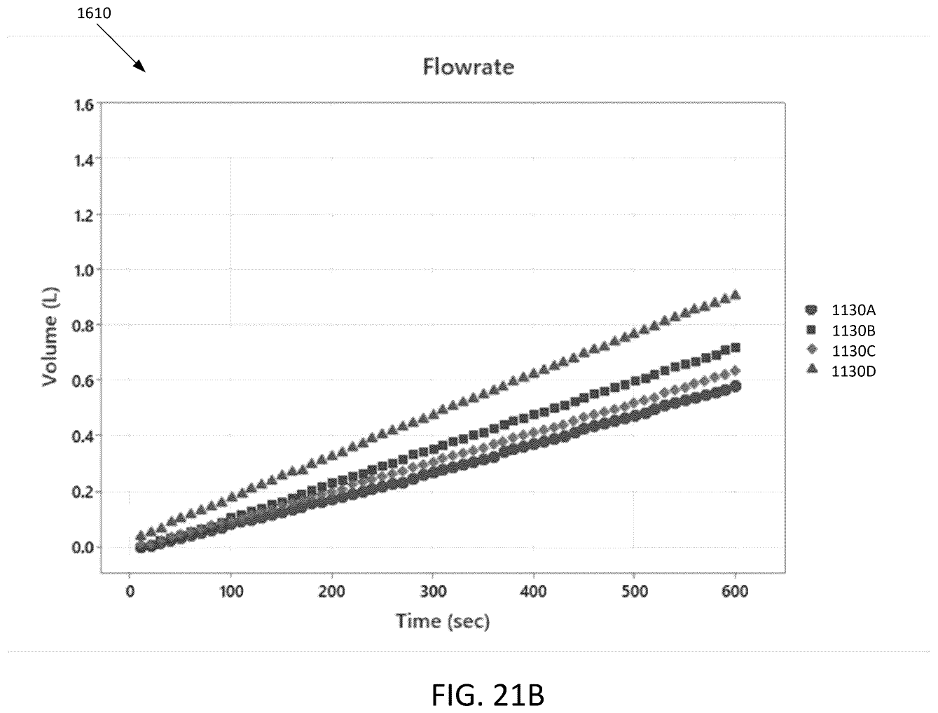

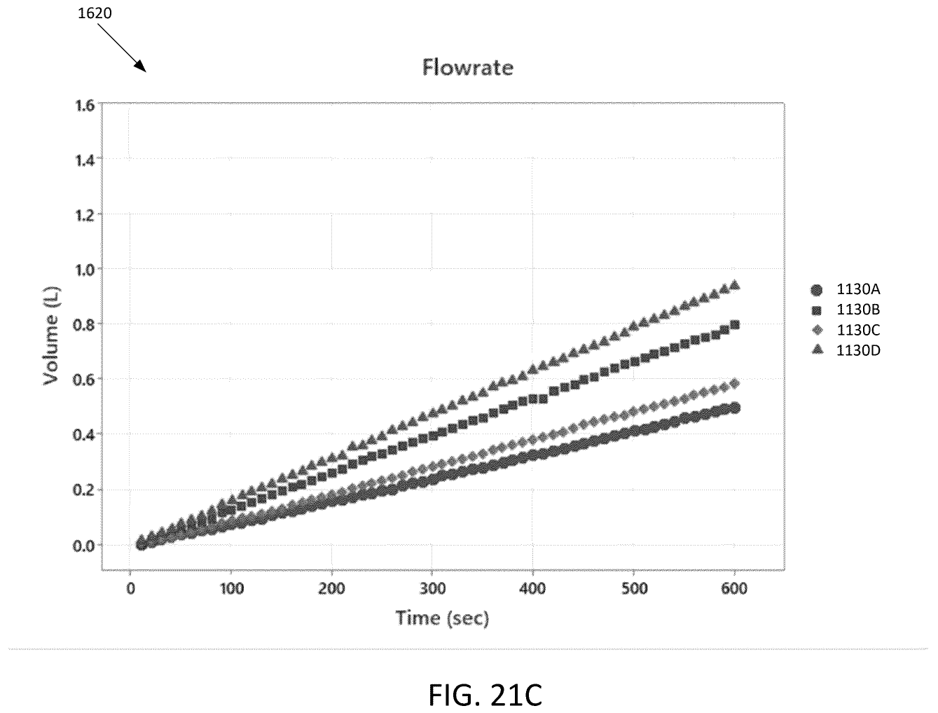

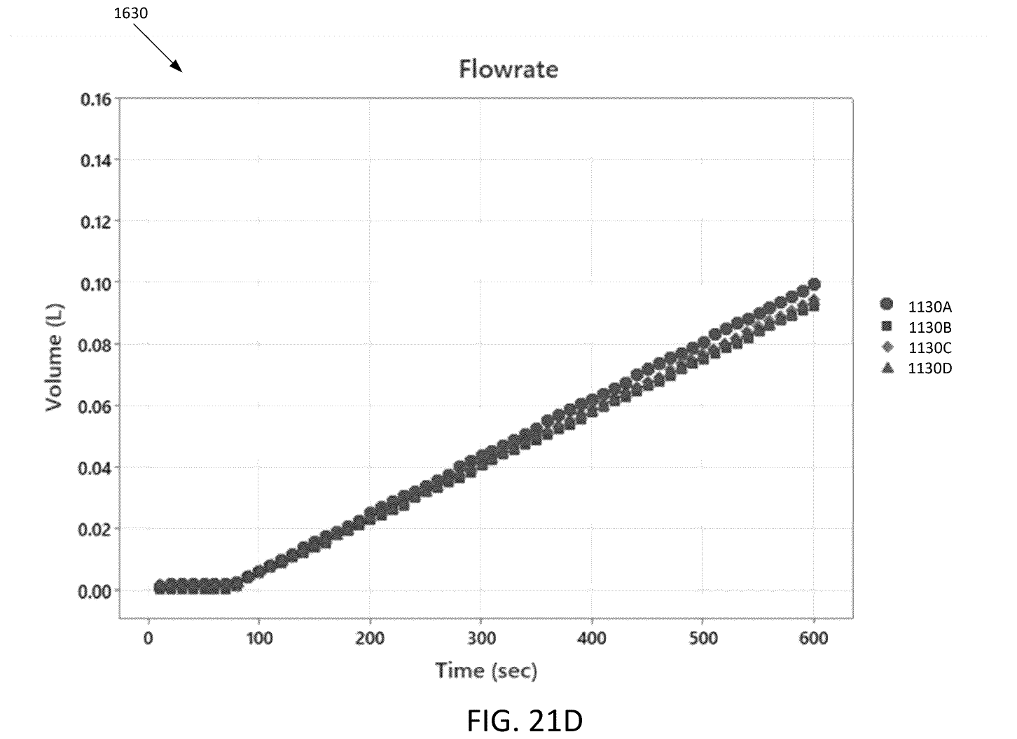

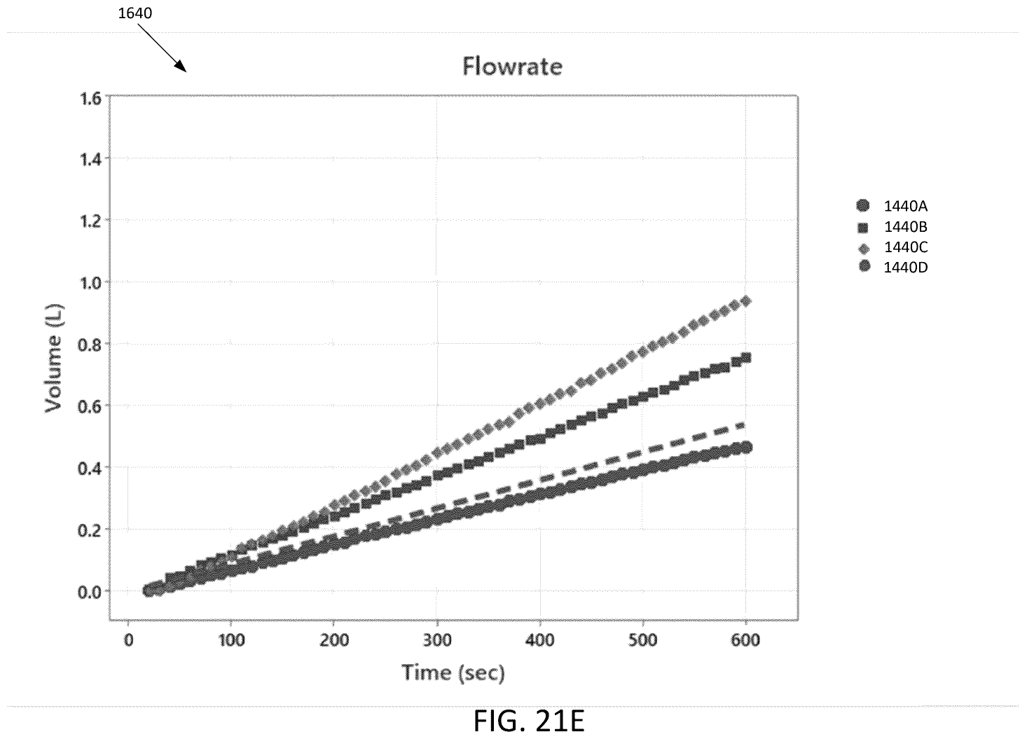

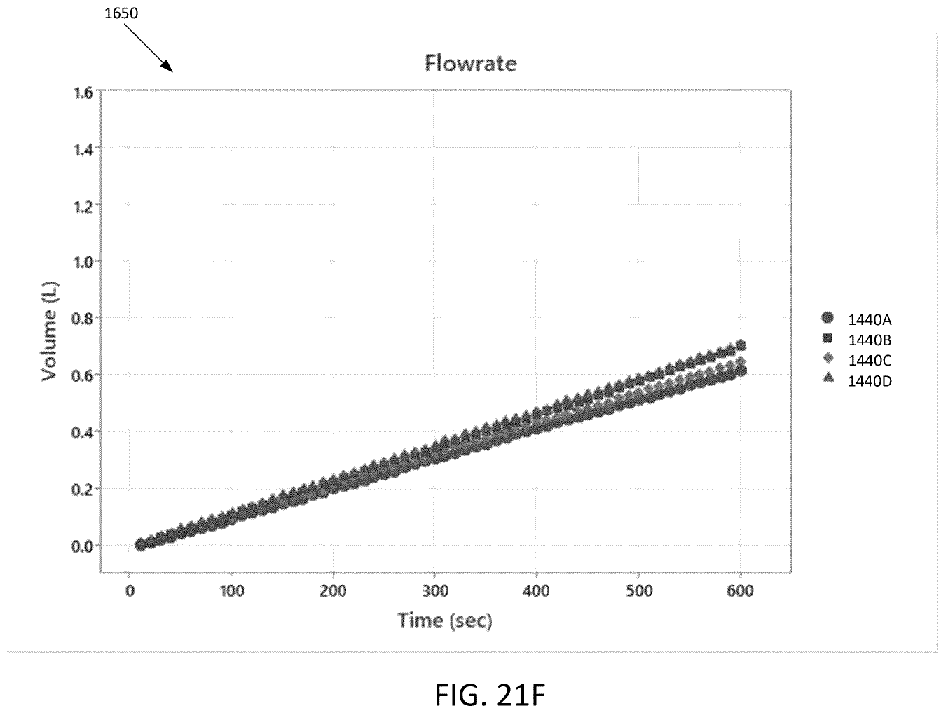

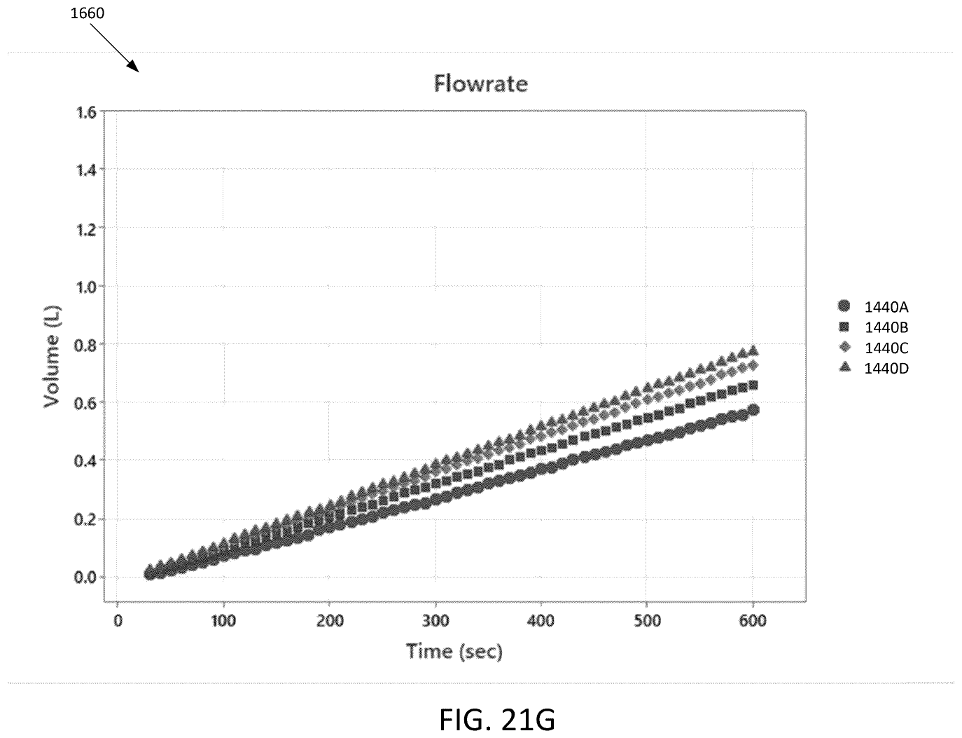

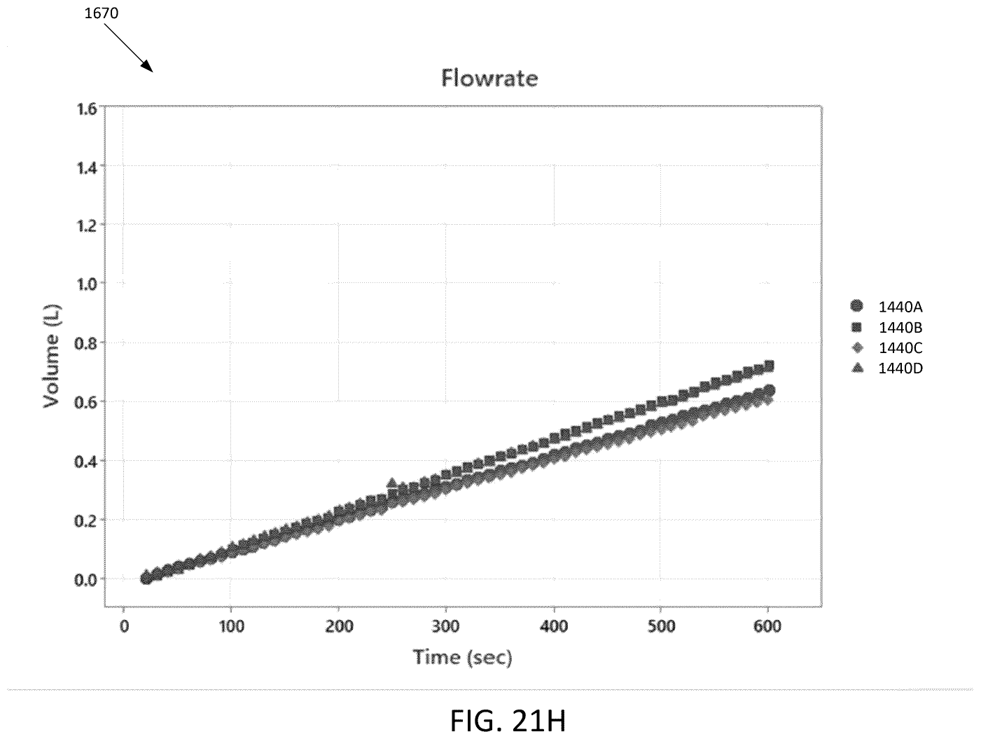

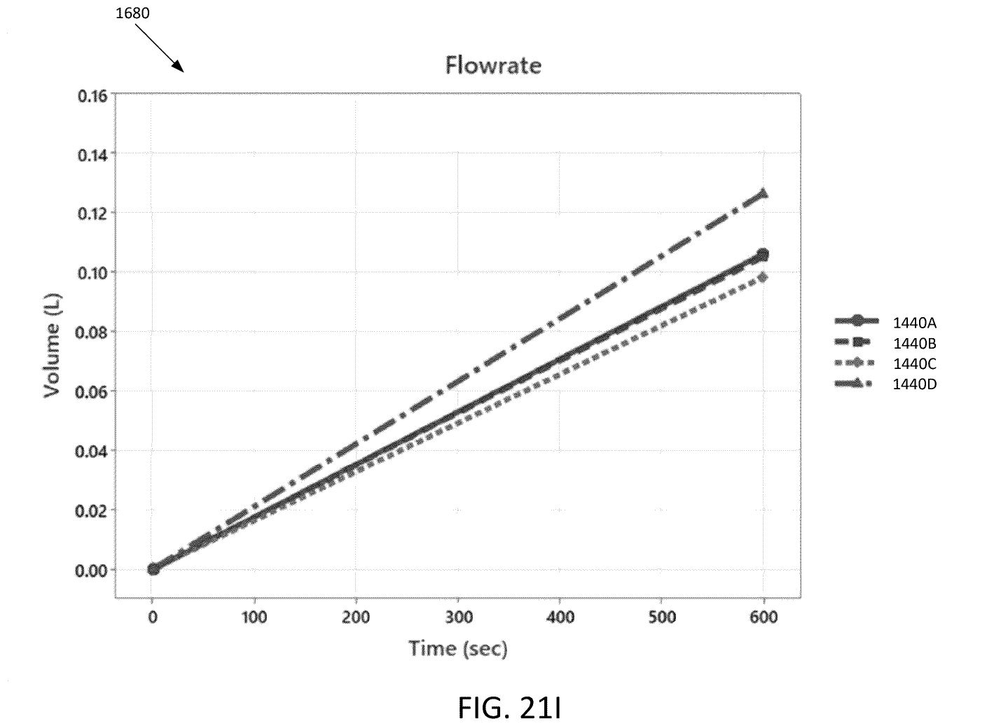

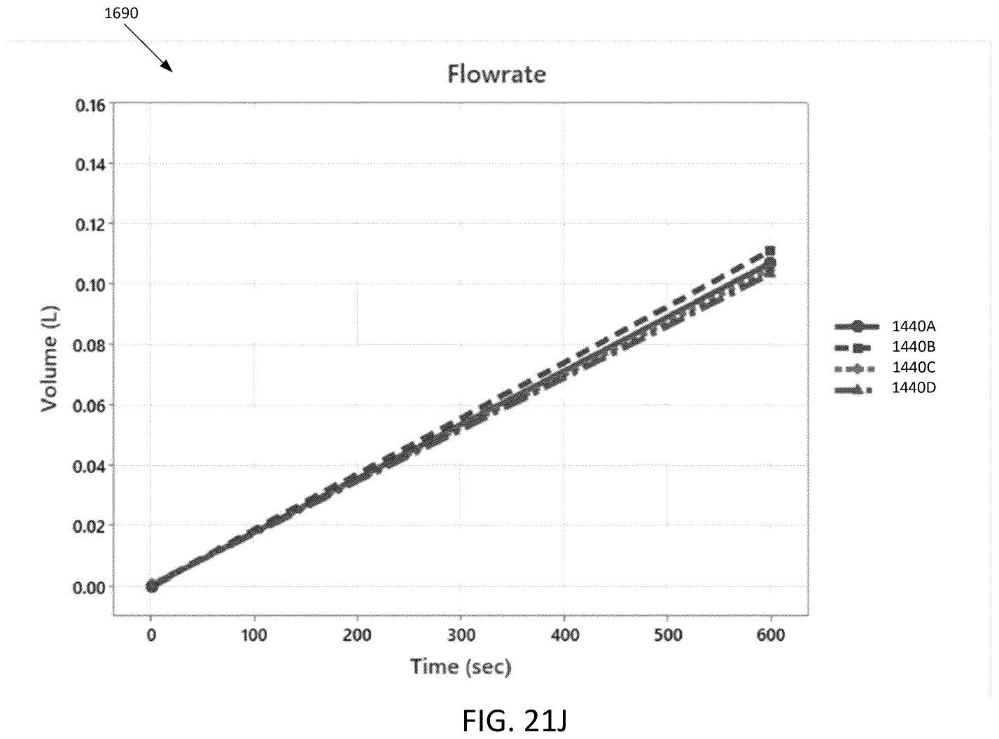

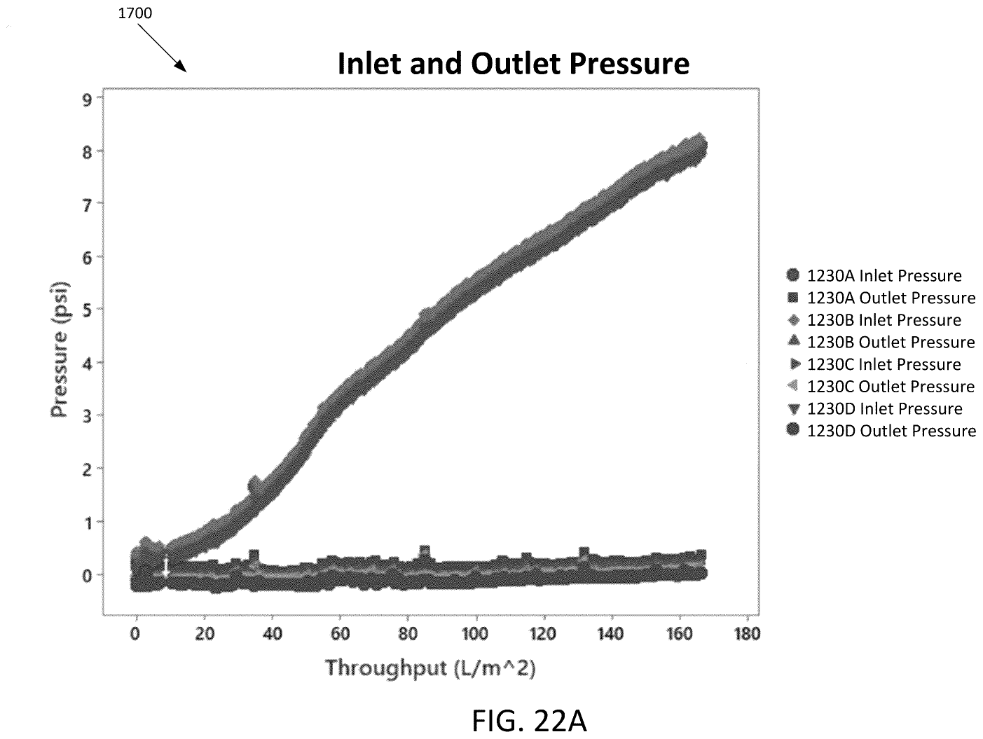

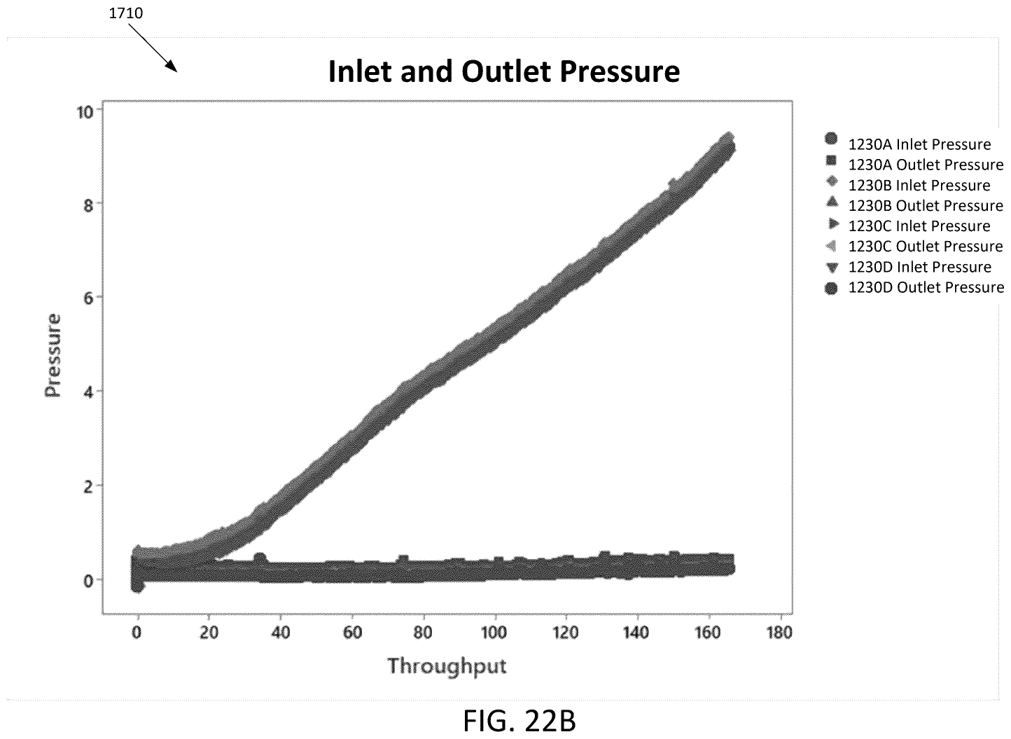

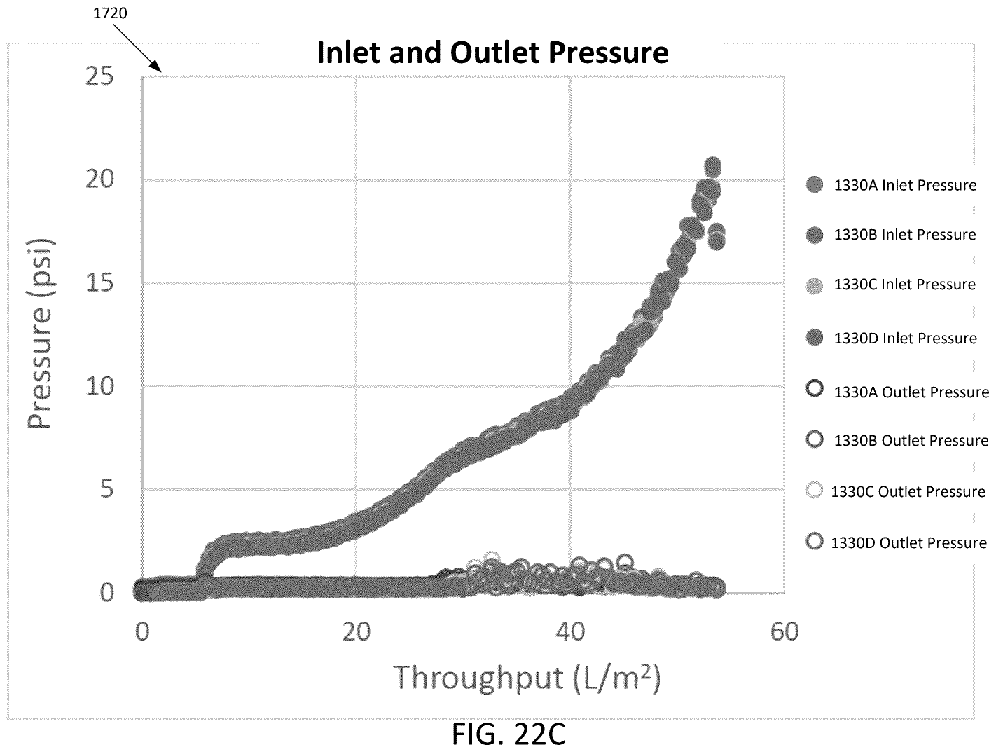

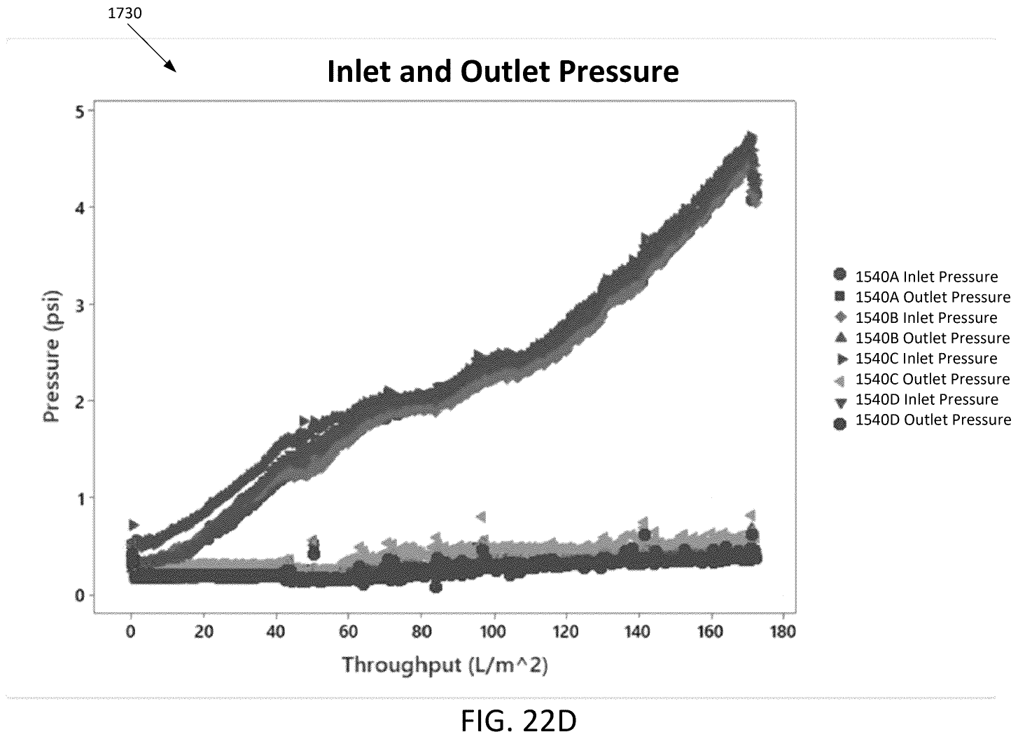

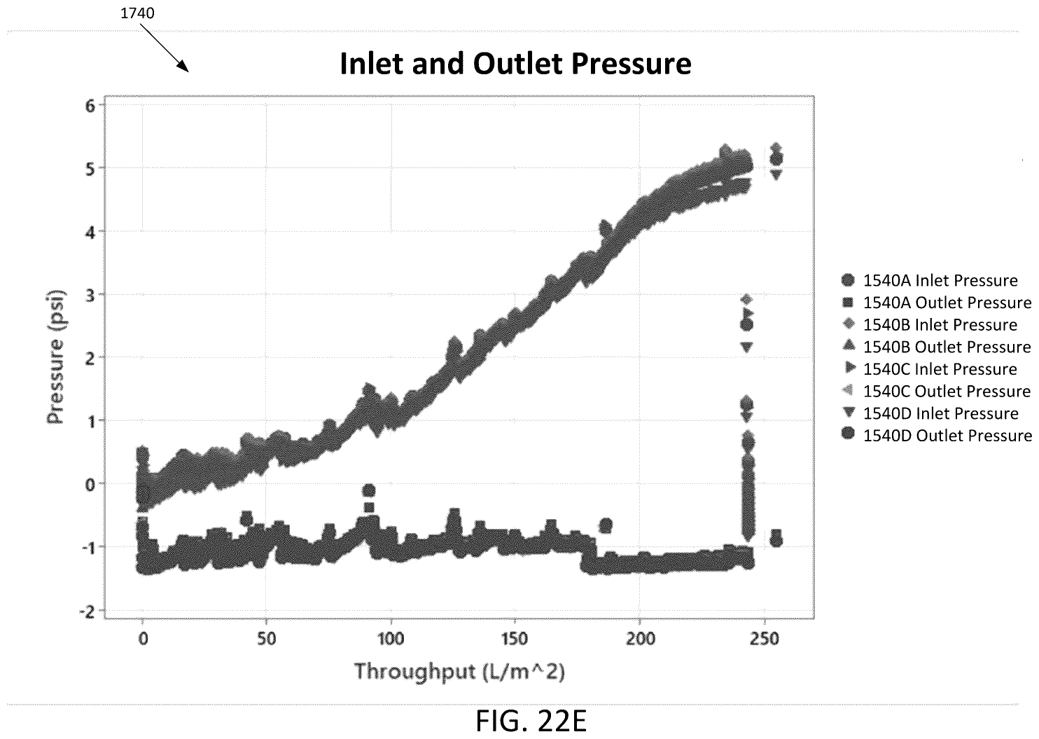

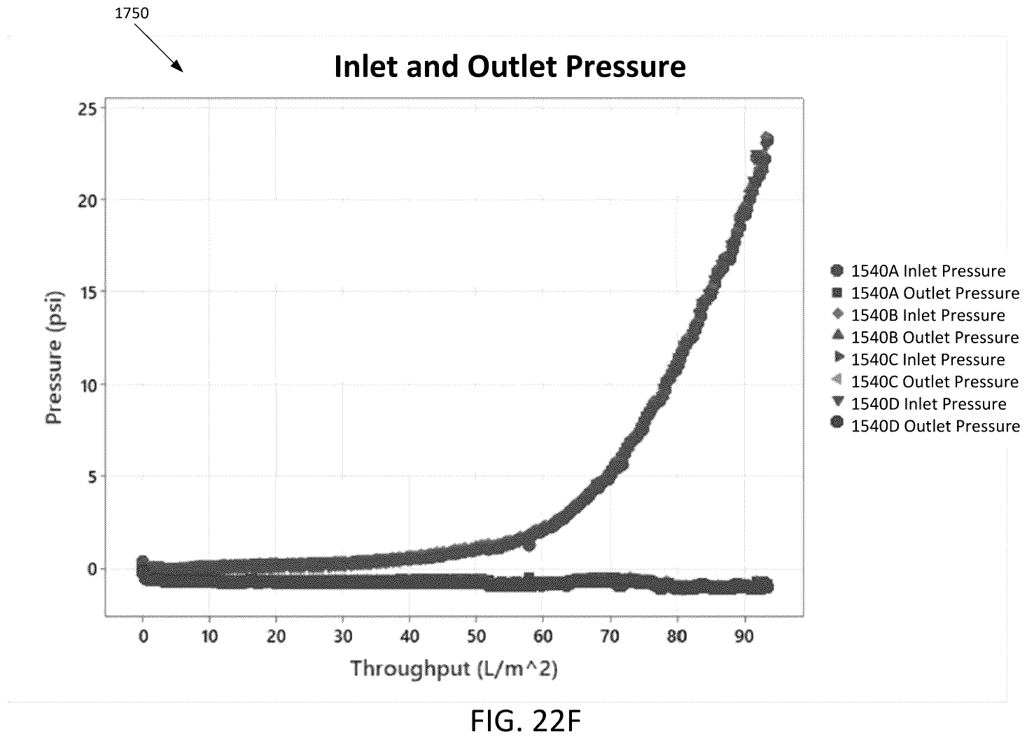

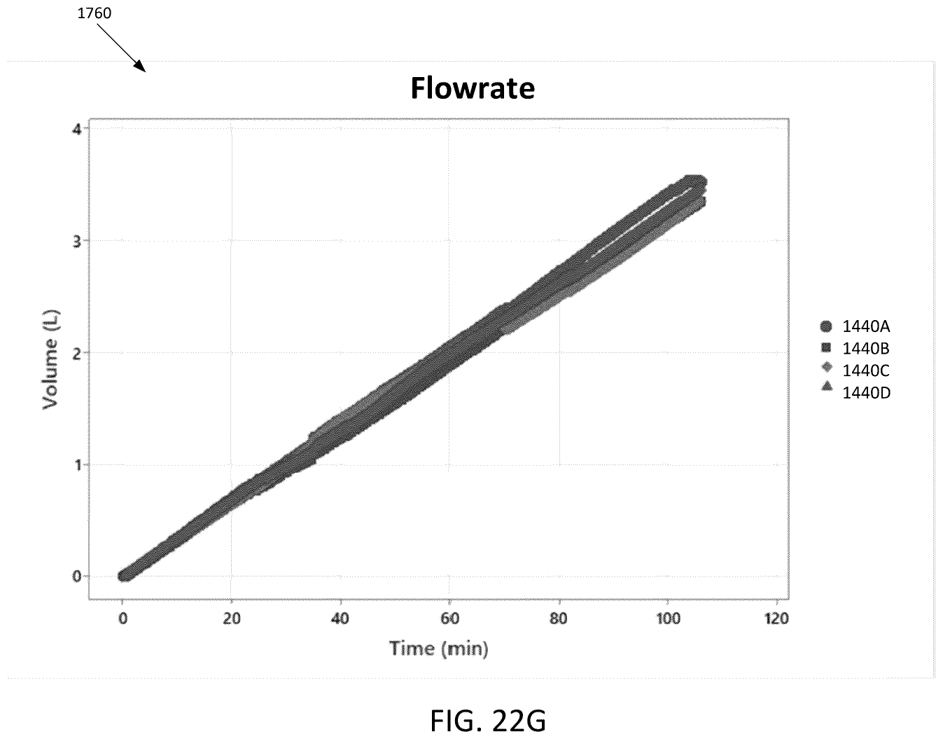

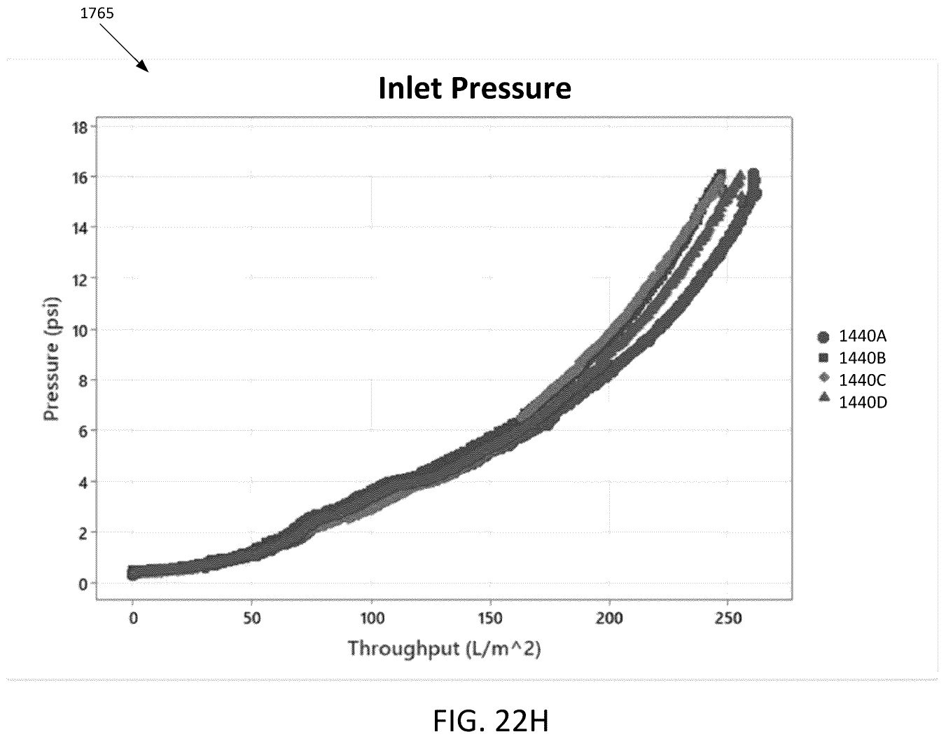

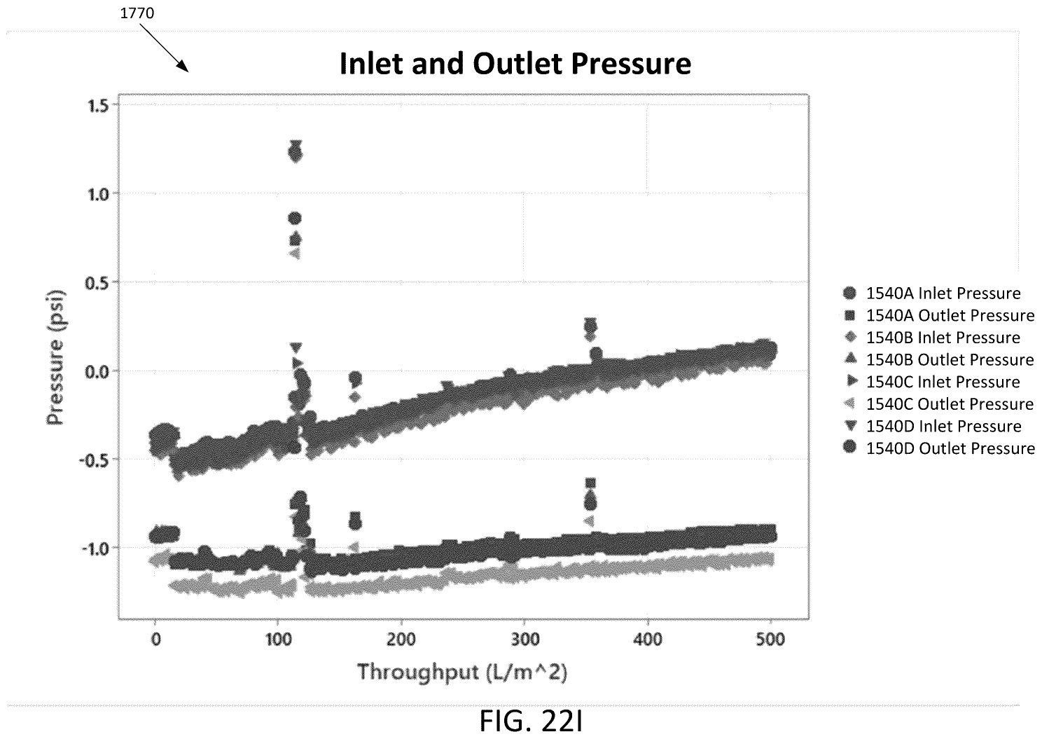

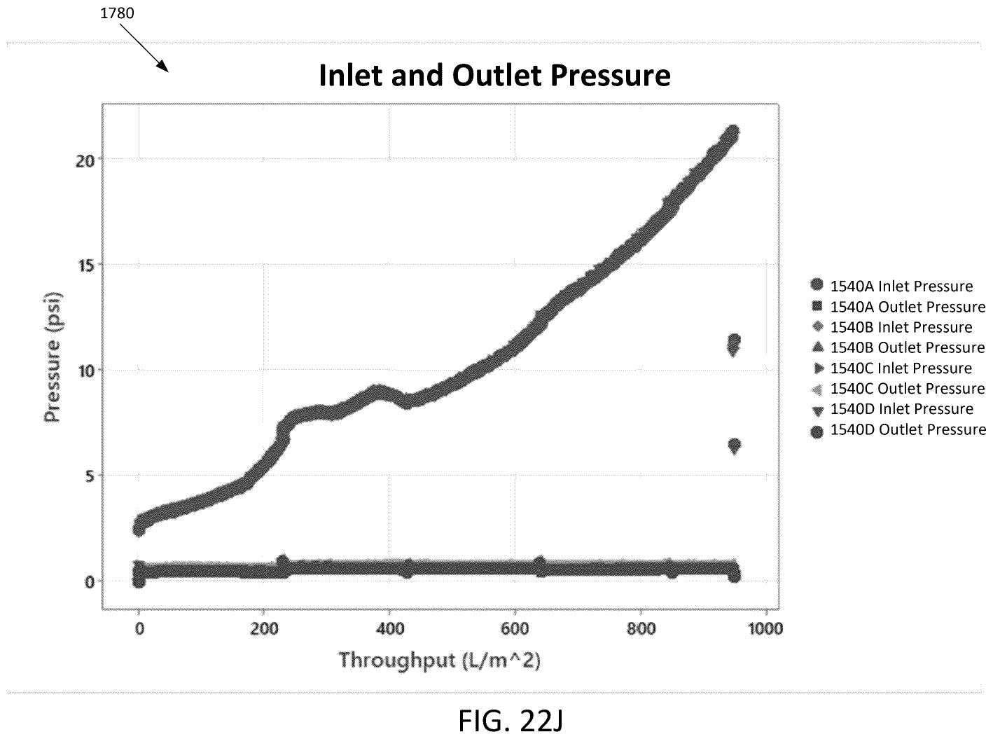

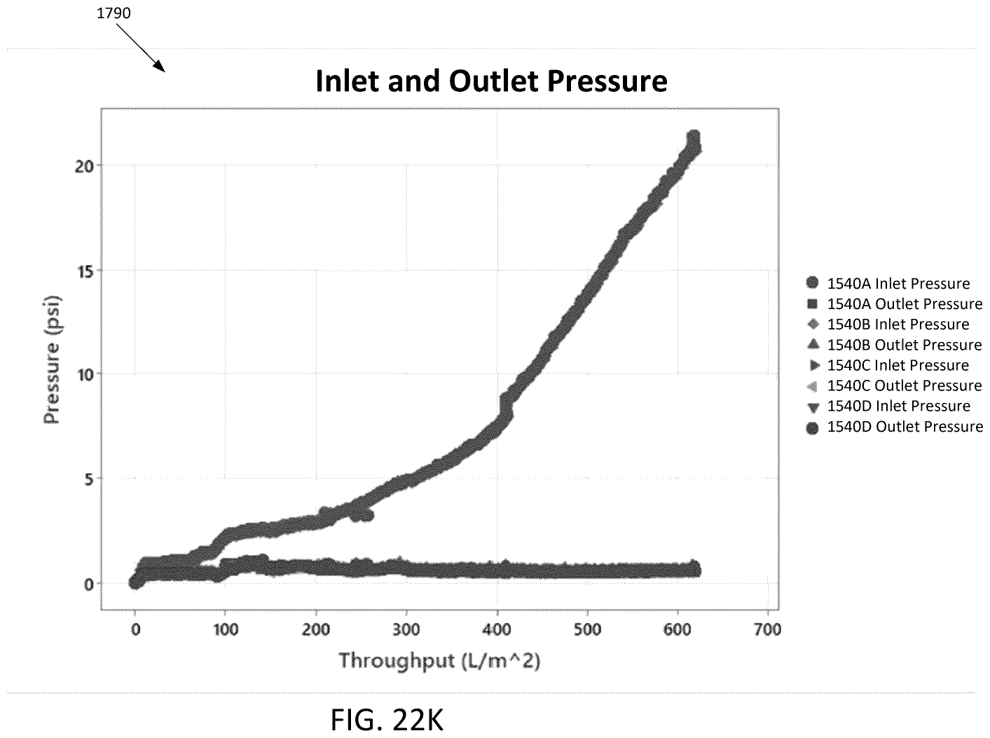

Turning to FIGS. 19A-19C and 20A-20B, illustrated are schematic depictions of manifolds 1100, 1200, 1300, 1400, 1500 that were tested and analyzed with various different grade pod-type filtrations devices and with conduits having various internal diameters in order to determine if certain manifold arrangements, including but not limited to flow arrangements (e.g., Linear, U-shaped, Z-shaped, bifurcated, etc.), conduit dimensions, etc., produce a uniform flow distribution between the pod-type filtration devices. FIGS. 21A-21J depict graphs that plot volume vs. time for each of the pod-filtration devices of certain manifolds arrangements when the manifolds receive a flushing feed stream in accordance with certain example experiments. FIGS. 22A-22K depict graphs that plot either pressure vs. throughput or volume vs. time for each of the pod-filtration devices of certain manifolds arrangements when the manifolds receive a clogging feed stream (i.e., conduct a filtration test run) in accordance with certain example experiments.

With reference to FIG. 19A, illustrated is a linear manifold 1100, which may be similar to the linear manifolds 300, 400 depicted in FIGS. 9 and 10, respectively. The linear manifold 1100 may include a main inlet conduit 1110 and a series of branch conduits 1120A-1120D (collectively referred to as 1120) extending from the main inlet conduit 1110. While FIG. 19A depicts the linear manifold 1100 with four branch conduits 1120A-1120D, a linear manifold may include any number of branch conduits 1120 greater than one. As further illustrated in FIG. 19A, the main inlet conduit 1110 may include a first end 1112, which serves as an inlet through which a feed stream may flow in order to enter the main inlet conduit 1110 and eventually flow into each of the branch conduits 1120A-1120D, and a second end 1114 opposite the first end 1112. Each of the branch conduits 1120A-1120D may extend from the same side of the main inlet conduit 1110, and may be equally spaced from one another along the main inlet conduit 1110 between the first end 1112 and the second end 1114. As illustrated, each branch conduit 1120A-1120D may include a first end 1122A-1122D (collectively referred to as 1122), respectively, and an opposite second end 1124A-1124D (collectively referred to as 1124), respectively. The first end 1122A-1122D of each branch conduit 1120A-1120D may be coupled to the main inlet conduit 1110, while the second end 1124A-1124D of each branch conduit 1120A-1120D may be coupled to a pod-type filtration device 1130A-1130D (collectively referred to as 1130). Each pod-type filtration device 1130A-1130D may be equipped with a pressure transducer 1132A-1132D, respectively, that is configured to monitor the inlet pressure to the respective pod-type filtration device 1130A-1130D. As further illustrated in FIG. 19A, connected to each of the pod-type filtration devices 1130A-1130D is an outlet conduit 1140A-1140D, respectively. Each outlet conduit 1140A-1140D includes a first end 1142A-1142D, which is coupled to a respective pod-type filtration device 1130A-1130D, and an opposite second end 1144A-1144D, which serves as an individual outlet for each pod-type filtration device 1130A-1130D. As detailed in the experimental scenarios outlined below, the bifurcated manifold 1100 illustrated in FIG. 19A was evaluated for various different conduit internal diameters and different pod-type filtration devices when the feed stream is a non-plugging feed stream.

Turning to FIG. 19B, illustrated is a linear Z-shaped manifold 1200, which may be similar to the Z-type manifold 200 depicted in FIGS. 8A and 8B. The linear Z-shaped manifold 1200 may include a main inlet conduit 1210 and a series of inlet branch conduits 1220A-1220D (collectively referred to as 1220) extending from the main inlet conduit 1210. While FIG. 19B depicts the linear Z-shaped manifold 1200 with four inlet branch conduits 1220A-1220D, a linear Z-shaped manifold may include any number of inlet branch conduits 1220 that is greater than one. As further illustrated in FIG. 19B, the main inlet conduit 1210 may include a first end 1212, which serves as an inlet through which a feed stream may flow in order to enter the linear Z-shaped manifold 1200 (e.g., enter the main inlet conduit 1210 and eventually flow into each of the inlet branch conduits 1220A-1220D, and a second end 1214 opposite the first end 1212). Each of the inlet branch conduits 1220A-1220D may extend from the same side of the main inlet conduit 1210, and may be equally spaced from one another along the main inlet conduit 1210 between the first end 1212 and the second end 1214. As illustrated, each inlet branch conduit 1220A-1220D may include a first end 1222A-1222D (collectively referred to as 1222), respectively, and an opposite second end 1224A-1224D (collectively referred to as 1224), respectively. The first end 1222A-1222D of each inlet branch conduit 1220A-1220D may be coupled to the main inlet conduit 1210, while the second end 1224A-1224D of each inlet branch conduit 1220A-1220D may be coupled to a pod-type filtration device 1230A-1230D (collectively referred to as 1230). Each pod-type filtration device 1230A-1230D is equipped with an inlet pressure transducer 1232A-1232D, respectively, that is configured to monitor the inlet pressure of the feed stream flow entering the respective pod-type filtration device 1230A-1230D.

As further illustrated in FIG. 19B, connected to each of the pod-type filtration devices 1230A-1230D is an outlet branch conduit 1240A-1240D (collectively referred to as 1240), respectively. Each outlet branch conduit 1240A-1240D includes a first end 1242A-1242D (collectively referred to as 1242), which is coupled to a respective pod-type filtration device 1230A-1230D, and an opposite second end 1244A-1244D (collectively referred to as 1244), respectively. The second end 1244A-1244D of each of the outlet branch conduits 1240A-1240D are coupled to a main outlet conduit 1250, which includes a first end 1252 and an opposite second end 1254. The second end 1244A-1244D of each of the outlet branch conduits 1240A-1240D are coupled to a main outlet conduit 1250 at equidistant locations from one another between the first and second ends 1252, 1254 of the main outlet conduit 1250. As depicted, each outlet branch conduit 1240A-1240D is also equipped with an outlet pressure transducer 1246A-1246D (collectively referred to as 1246), respectively, that is configured to monitor the outlet pressure of the feed stream flow exiting the respective pod-type filtration device 1230A-1230D. Furthermore, the second end 1254 of the main outlet conduit 1250 serves as the outlet of the linear Z-shaped manifold 1200 through which a feed stream may flow in order to exit the linear Z-shaped manifold 1200. The flow of the feed stream into the inlet (e.g., the first end 1212 of the main inlet conduit 1210) of the linear Z-shaped manifold 1200 is in the same direction as that of the flow of the feed stream out of the outlet (e.g., the second end 1254 of the main outlet conduit 1250) of the linear Z-shaped manifold 1200. As detailed in the experimental scenarios outlined below, the linear Z-shaped manifold 1200 illustrated in FIG. 19B was evaluated for various different conduit internal diameters and different pod-type filtration devices when the feed stream is a plugging feed stream.

Turning to FIG. 19C, illustrated is a linear U-shaped manifold 1300, which may be similar to the U-type manifold 100 depicted in FIGS. 7A and 7B. Like that of the linear manifold 1100 and the linear Z-shaped manifold 1200, the linear U-shaped manifold 1300 may include a main inlet conduit 1310 and a series of inlet branch conduits 1320A-1320D (collectively referred to as 1320) extending from the main inlet conduit 1310. While FIG. 19C depicts the linear U-shaped manifold 1300 with four inlet branch conduits 1320A-1320D, a linear U-shaped manifold may include any number of inlet branch conduits 1320 that is greater than one. As further illustrated in FIG. 19C, the main inlet conduit 1310 may include a first end 1312, which serves as an inlet through which a feed stream may flow in order to enter the linear U-shaped manifold 1300 (e.g., enter the main inlet conduit 1310 and eventually flow into each of the inlet branch conduits 1320A-1320D, and a second end 1314 opposite the first end 1312). Each of the inlet branch conduits 1320A-1320D may extend from the same side of the main inlet conduit 1310, and may be equally spaced from one another along the main inlet conduit 1310 between the first end 1312 and the second end 1314. As illustrated, each inlet branch conduit 1320A-1320D may include a first end 1322A-1322D (collectively referred to as 1322), respectively, and an opposite second end 1324A-1324D (collectively referred to as 1324), respectively. The first end 1322A-1322D of each inlet branch conduit 1320A-1320D may be coupled to the main inlet conduit 1310, while the second end 1324A-1324D of each inlet branch conduit 1320A-1320D may be coupled to a pod-type filtration device 1330A-1330D (collectively referred to as 1330). Each pod-type filtration device 1330A-1330D is equipped with an inlet pressure transducer 1332A-1332D, respectively, that is configured to monitor the inlet pressure of the feed stream flow entering the respective pod-type filtration device 1330A-1330D.

As further illustrated in FIG. 19C, connected to each of the pod-type filtration devices 1330A-1330D is an outlet branch conduit 1340A-1340D (collectively referred to as 1340), respectively. Each outlet branch conduit 1340A-1340D includes a first end 1342A-1342D (collectively referred to as 1342), which is coupled to a respective pod-type filtration device 1330A-1330D, and an opposite second end 1344A-1344D (collectively referred to as 1344). The second end 1344A-1344D of each of the outlet branch conduits 1340A-1340D is coupled to a main outlet conduit 1350, which includes a first end 1352 and an opposite second end 1354. The second end 1344A-1344D of each of the outlet branch conduits 1340A-1340D are coupled to a main outlet conduit 1350 at equidistant locations from one another at locations between the first and second ends 1352, 1354 of the main outlet conduit 1350. As depicted, each outlet branch conduit 1340A-1340D is also equipped with an outlet pressure transducer 1346A-1346D (collectively referred to as 1346), respectively, that is configured to monitor the outlet pressure of the feed stream flow exiting the respective pod-type filtration device 1330A-1330D. Furthermore, and opposite of that of the linear Z-shaped manifold 1200, the first end 1352 of the main outlet conduit 1350 serves as the outlet of the linear U-shaped manifold 1300 through which a feed stream may flow in order to exit the linear U-shaped manifold 1300. The flow of the feed stream into the inlet (e.g., the first end 1312 of the main inlet conduit 1310) of the linear U-shaped manifold 1300 is in the opposite direction as that of the flow of the feed stream out of the outlet (e.g., the first end 1352 of the main outlet conduit 1350) of the linear U-shaped manifold 1300. As detailed in the experimental scenarios outlined below, the linear U-shaped manifold 1300 illustrated in FIG. 19C was evaluated for various different conduit internal diameters and different pod-type filtration devices when the feed stream is a plugging feed stream.

With reference to FIG. 20A, illustrated is a bifurcated manifold 1400, which may be similar to the bifurcated manifold 600 depicted in FIGS. 13A and 13B. As illustrated in FIG. 20A, the bifurcated manifold 1400 includes main inlet conduit 1410 having a first end 1412 and an opposite second end 1414, where the first end 1412 may serve as an inlet for a feed stream to enter the bifurcated manifold 1400. The bifurcated manifold 1400 may further include any number of tiered inlet branch conduits to support the symmetrical bifurcation of the feed stream through the bifurcated manifold 1400. In the example embodiment illustrated in FIG. 20A, the bifurcated manifold 1400 includes a primary inlet branch conduit 1420 that is coupled to the main inlet conduit 1410 at the second end 1414 of the main inlet conduit 1410, and four secondary inlet branch conduits 1430A-1430D (collectively referred to as 1430) that are each coupled to the primary inlet branch conduit 1420. The primary inlet branch conduit 1420 may include a first end 1422 and an opposite second end 1424, where the second end 1414 of the main inlet conduit 1410 is coupled to the primary inlet branch conduit 1420 between the first and second ends 1422, 1424 of the primary inlet branch conduit 1420. As further illustrated in FIG. 20A, each of the secondary inlet branch conduits 1430 include a first end 1432A-1432D (collectively referred to as 1432) and an opposite second end 1434A-1434D (collectively referred to as 1434). The first ends 1432A-1432D of the secondary inlet branch conduits 1430A-1430D are coupled to the primary inlet branch conduit 1420, while each of the second ends 1434 of the secondary inlet branch conduits 1430 is coupled to a pod-type filtration device or pod 1440A-1440D (collectively referred to as 1440), respectively. More specifically, the first ends 1432A, 1432C of the first and third secondary inlet branch conduits 1430A, 1430C, respectively, are coupled to the first end 1422 of the primary inlet branch conduit 1420, while the first ends 1432B, 1432D of the second and fourth secondary inlet branch conduits 1430B, 1430D, respectively, are coupled to the second end 1424 of the primary inlet branch conduit 1420.

As further illustrated in FIG. 20A, each pod-type filtration device 1440A-1440D may be equipped with an inlet pressure transducer 1442A-1442D (collectively referred to as 1442), respectively, that is configured to monitor the inlet pressure to the respective pod-type filtration device 1440A-1440D. FIG. 20A further depicts an outlet conduit 1450A-1450D (collectively referred to as 1450) connected to each of the pod-type filtration devices 1440A-1440D, respectively. Each outlet conduit 1450A-1450D includes a first end 1452A-1452D (collectively referred to as 1452), which is coupled to a respective pod-type filtration device 1440A-1440D, and an opposite second end 1454A-1454D (collectively referred to as 1454), which serves as an individual outlet for each pod-type filtration device 1440A-1440D. As detailed in the experimental scenarios outlined below, the bifurcated manifold 1400 illustrated in FIG. 20A was evaluated for various different conduit internal diameters and different pod-type filtration devices when the feed stream is a non-plugging feed stream.

Turning to FIG. 20B, illustrated is a bifurcated manifold 1500, which may be similar to the bifurcated manifold 600 depicted in FIGS. 13A and 13B. The bifurcated manifold 1500 of FIG. 20B may be identical to the bifurcated manifold 1400 of FIG. 20A except that the bifurcated manifold 1500 includes outlet branch conduits that converge with one another to form a single outlet for the bifurcated manifold 1500. As illustrated in FIG. 20B, and similar to the bifurcated manifold 1400, the bifurcated manifold 1500 includes main inlet conduit 1510 having a first end 1512 and an opposite second end 1514, where the first end 1512 serves as an inlet for a feed stream to enter the bifurcated manifold 1500. The bifurcated manifold 1500 may further include any number of tiered inlet branch conduits to support the symmetrical bifurcation of the feed stream through the bifurcated manifold 1500. In the example embodiment illustrated in FIG. 20B, the bifurcated manifold 1500 includes a primary inlet branch conduit 1520 that is coupled to the main inlet conduit 1510 at the second end 1514 of the main inlet conduit 1510, and four secondary inlet branch conduits 1530A-1530D (collectively referred to as 1530) that are each coupled to the primary inlet branch conduit 1520. The primary inlet branch conduit 1520 may include a first end 1522 and an opposite second end 1524, where the second end 1514 of the main inlet conduit 1510 is coupled to the primary inlet branch conduit 1520 between the first and second ends 1522, 1524 of the primary inlet branch conduit 1520. As further illustrated in FIG. 20A, each of the secondary inlet branch conduits 1530 include a first end 1532A-1532D (collectively referred to as 1532) and an opposite second end 1534A-1534D (collectively referred to as 1534). The first ends 1532A-1532D of the secondary inlet branch conduits 1530A-1530D are coupled to the primary inlet branch conduit 1520, while each of the second ends 1534 of the secondary inlet branch conduits 1530 is coupled to a pod-type filtration device or pod 1540A-1540D (collectively referred to as 1440), respectively. More specifically, the first ends 1532A, 1532C of the first and third secondary inlet branch conduits 1530A, 1530C, respectively, are coupled to the first end 1522 of the primary inlet branch conduit 1520, while the first ends 1532B, 1532D of the second and fourth secondary inlet branch conduits 1530B, 1530D, respectively, are coupled to the second end 1524 of the primary inlet branch conduit 1520.

As further illustrated in FIG. 20B, each pod-type filtration device 1540A-1540D may be equipped with an inlet pressure transducer 1542A-1542D (collectively referred to as 1542), respectively, that is configured to monitor the inlet pressure to the respective pod-type filtration device 1540A-1540D. Furthermore, connected to each of the pod-type filtration devices 1540A-1540D is a secondary outlet branch conduit 1550A-1550D (collectively referred to as 1550), respectively. Each secondary outlet branch conduit 1550A-1550D includes a first end 1552A-1552D (collectively referred to as 1552), which is coupled to a respective pod-type filtration device 1540A-1540D, and an opposite second end 1554A-1554D (collectively referred to as 1554). The second end 1554A-1554D of each secondary outlet branch conduit 1550A-1550D is coupled to a primary outlet branch conduit 1560. The primary outlet branch conduit 1560 includes a first end 1562 and an opposite second end 1564. The second ends 1554A, 1554C of the first and third secondary outlet branch conduits 1550A, 1550C, respectively, are coupled to the first end 1562 of the primary outlet branch conduit 1560, while the second ends 1554B, 1554D of the second and fourth secondary outlet branch conduits 1550B, 1550D, respectively, are coupled to the second end 1564 of the primary outlet branch conduit 1560. A main outlet conduit 1570 having a first end 1572 and an opposite second end 1574 may be coupled to the primary outlet branch conduit 1560, where the first end 1572 of the main outlet conduit 1570 is coupled to the primary outlet branch conduit 1560 at a location between the first and second ends 1562, 1564 of the primary outlet branch conduit 1560. The second end 1574 of the main outlet conduit 1570 may serve as the outlet of the bifurcated manifold 1500. As detailed in the experimental scenarios outlined below, the bifurcated manifold 1500 illustrated in FIG. 20A was evaluated for various different conduit internal diameters and different pod-type filtration devices when the feed stream is a plugging feed stream.

Turning to FIGS. 21A-21J, and with continued reference to FIGS. 19A and 20A, illustrated are graphs that depict the outcome of various experiments run on the linear manifold 1100 and the bifurcated manifold 1400 by flowing a non-plugging feed stream (e.g., water) through the two manifolds 1100, 1400. In total, and as depicted in Table 1 below, ten experiments were run using the linear manifold 1100 and the bifurcated 1400, where four of the experiments were run on the linear manifold 1100 and six of the experiments were run on the bifurcated manifold 1400. The internal diameters of the inlet conduits (i.e., including the branch inlet conduits) were varied in accordance with that depicted in Table 1, as well as the filter grade and filter size. Thus, the flow resistance of pathways through the manifolds were altered between the experiments. In addition, all of the outlet tubing for the experiments were sized by the filter type used, where the internal diameter of the outlet tubing for lab scale filters was approximately 0.25 inches (¼″) and the internal diameter of the outlet tubing for the micro scale filters was approximately 0.0625 inches ( 1/16″). As depicted in Table 1, three different filters media grades were tested. The DOSP filter is a depth filter having a media grade that comprises a non-woven layer and a coarse grade of silica filter aid with polyacrylic fiber (having a larger nominal pore size rating). The XOSP filter is a depth filter having a tighter grade of silica filter aid with polyacrylic fiber (having a smaller nominal pore size rating). The 60HX filter is a depth filter having a series of polypropylene felt materials arranged in manner to form a gradient of nominal pore size ratings arranged in a manner of decreasing nominal pore size from the device inlet to the outlet.

TABLE 1
Pre-Use Flushing Experiments with Non-Plugging Feed Streams

| | | Main | Primary | Secondary | | | | |
|---|---|---|---|---|---|---|---|---|
| Experimental | Manifold | Inlet | Inlet | Inlet | Filter | | | Feed |
| Run | Type | Conduit ID | Branch ID | Branch ID | Grade | Filter Size | Outlet | Stream |
| 1 | Linear | ¼″ | ¼″ | N/A | D0SP | 135 LSP | Individual | Water |
| 2 | Linear | ½″ | ¼″ | N/A | D0SP | 135 LSP | Individual | Water |
| 3 | Linear | ½″ | ⅛″ | N/A | D0SP | 135 LSP | Individual | Water |
| 4 | Linear | ⅛″ | 1/16″ | N/A | X0SP | Micro 20 | Individual | Water |
| 5 | Bifurcated | ¼″ | ¼″ | ¼″ | D0SP | 135 LSP | Individual | Water |
| 6 | Bifurcated | ¼″ | ⅛″ | 1/16″ | D0SP | 135 LSP | Individual | Water |
| 7 | Bifurcated | ¼″ | ⅛″ | 1/16″ | 60HX | 135 LSP | Individual | Water |
| 8 | Bifurcated | ¼″ | ⅛″ | 1/16″ | X0SP | 135 LSP | Individual | Water |
| 9 | Bifurcated | ⅛″ | 1/16″ | 1/32″ | X0SP | Micro 20 | Individual | Water |
| 10 | Bifurcated | ⅛″ | 1/16″ | 1/32″ | D0SP | Micro 20 | Individual | Water |

Turning to FIG. 21A, illustrated is a graph 1600 of a plot of the volume experienced by each of the pod-type filter devices 1130A-1130D of the linear manifold 1100 in accordance with the manifold arrangement of experimental run 1 depicted in Table 1. More specifically, the linear manifold 1100 was evaluated where the main inlet conduit 1110 and the branch inlet conduits 1120A-1120D all had an internal diameter of approximately 0.25 inches. In addition, each of the branch inlet conduits 1120A-1120D were equal in length. The pod-type filtration devices 1130A-1130D coupled to the branch inlet conduits 1120A-1120D were DOSP grade filters having a filter size or surface area of approximately 135 cm² (cubic centimeters). A pump was used to flush deionized water through the linear manifold 1100 at a targeted flowrate of 300 liters per square meter per hour (LMH) for at least 50 liters per square meter (L/m²). The feed stream water was collected individually from the four outlet conduits 1140A-1140D into buckets with scales. The filter pressure and flush volume were continuously recorded by means of a data recorder. As depicted in the graph 1600 in FIG. 21A, which is a plot of the volume received by each of the pod-type filtration devices 1130A-1130D versus time for the arrangement of the linear manifold 1100 as depicted in Table 1, a large variation exists between the volume measured at each of the pod-type filtration devices 1130A-1130D. Moreover, as demonstrated in Table 2 below, which depicts the flowrates measured for each of the pod-type filtration devices 1130A-1130D, a large variation exists (e.g., approximately 227 LMH) between the fastest and slowest flowrates measured at the pod-type filtration devices 1130A-1130D. More specifically, the second pod-type filtration device 1130B received the largest flowrate and a greater share of the flow distribution of the non-plugging feed stream, while the third pod-type filtration device 1130C received the lowest flowrate and the lowest share of the flow distribution of the non-plugging feed stream. Thus, according to the data from the first experiment, a uniform flow distribution of a non-plugging feed stream could not be achieved for the arrangement of the linear manifold 1100 depicted in Table 1 for the first experimental run.

TABLE 2
Experimental Run 1 Individual Flowrates

| 1130A | 1130B | 1130C | 1130D |
|---|---|---|---|
| 321 LMH | 477 LMH | 250 LMH | 389 LMH |

Turning to FIG. 21B, illustrated is a graph 1610 of a plot of the volume experienced by each of the pod-type filter devices 1130A-1130D of the linear manifold 1100 in accordance with the manifold arrangement of experimental run 2 depicted in Table 1 above. More specifically, the linear manifold 1100 was evaluated where the main inlet conduit 1110 had an internal diameter of approximately 0.5 inches and the branch inlet conduits 1120A-1120D all had an internal diameter of approximately 0.25 inches. In addition, each of the branch inlet conduits 1120A-1120D were equal in length. The pod-type filtration devices 1130A-1130D coupled to the branch inlet conduits 1120A-1120D were DOSP grade filters having a filter size or surface area of approximately 135 cm². Like that of the first experimental run, a pump was used to flush deionized water through the linear manifold 1100 at a targeted flowrate of 300 LMH for at least 50 L/m². The feed stream water was collected individually from the four outlet conduits 1140A-1140D into buckets with scales. The filter pressure and flush volume were continuously recorded by means of a data recorder. As depicted in the graph 1610 in FIG. 21B, which is a plot of the volume received by each of the pod-type filtration devices 1130A-1130D versus time for the arrangement of the linear manifold 1100 as depicted in Table 1, a large variation exists between the volume measured at each of the pod-type filtration devices 1130A-1130D. Moreover, as demonstrated in Table 3 below, which depicts the flowrates measured for each of the pod-type filtration devices 1130A-1130D, a large variation exists (e.g., approximately 143 LMH) between the fastest and slowest flowrates measured at the pod-type filtration devices 1130A-1130D. More specifically, the fourth pod-type filtration device 1130D received the largest flowrate and a greater share of the flow distribution of the non-plugging feed stream, while the first pod-type filtration device 1130A received the lowest flowrate and the lowest share of the flow distribution of the non-plugging feed stream. Thus, according to the data from the second experiment, while the second experimental run demonstrates that varying the inner diameters of the conduits from those of the first experimental run may reduce the variation between the flowrates and flow distribution, a uniform flow distribution of a non-plugging feed stream was still not achieved for the arrangement of the linear manifold 1100 depicted in Table 1 for the second experimental run.

TABLE 3
Experimental Run 2 Individual Flowrates

| 1130A | 1130B | 1130C | 1130D |
|---|---|---|---|
| 258 LMH | 319 LMH | 282 LMH | 401 LMH |

Turning to FIG. 21C, illustrated is a graph 1620 of a plot of the volume experienced by each of the pod-type filter devices 1130A-1130D of the linear manifold 1100 in accordance with the manifold arrangement of experimental run 3 depicted in Table 1 above. More specifically, the linear manifold 1100 was evaluated where the main inlet conduit 1110 had an internal diameter of approximately 0.5 inches and the branch inlet conduits 1120A-1120D all had an internal diameter of approximately 0.125 inches. In addition, each of the branch inlet conduits 1120A-1120D were equal in length. The pod-type filtration devices 1130A-1130D coupled to the branch inlet conduits 1120A-1120D were DOSP grade filters having a filter size or surface area of approximately 135 cm². Like that of the first and second experimental runs, a pump was used to flush deionized water through the linear manifold 1100 at a targeted flowrate of 300 LMH for at least 50 L/m², where the feed stream water was collected individually from the four outlet conduits 1140A-1140D into buckets with scales. In addition, the filter pressure and flush volume were continuously recorded by means of a data recorder. As depicted in the graph 1620 in FIG. 21C, which is a plot of the volume received by each of the pod-type filtration devices 1130A-1130D versus time for the arrangement of the linear manifold 1100 as depicted in Table 1, a large variation exists between the volume measured at each of the pod-type filtration devices 1130A-1130D. Moreover, as demonstrated in Table 4 below, which depicts the flowrates measured for each of the pod-type filtration devices 1130A-1130D, a large variation exists (e.g., approximately 195 LMH) between the fastest and slowest flowrates measured at the pod-type filtration devices 1130A-1130D. More specifically, the fourth pod-type filtration device 1130D received the largest flowrate and a greater share of the flow distribution of the non-plugging feed stream, while the first pod-type filtration device 1130A received the lowest flowrate and the lowest share of the flow distribution of the non-plugging feed stream. Thus, according to the data from the third experimental run, while further varying the inner diameters of the conduits reduces the variation between the flowrates and flow distribution when compared to the first and second experimental runs, a uniform flow distribution of a non-plugging feed stream was still not achieved for the arrangement of the linear manifold 1100 depicted in Table 1 for the third experimental run.

TABLE 4
Experimental Run 3 Individual Flowrates

| 1130A | 1130B | 1130C | 1130D |
|---|---|---|---|
| 221 LMH | 355 LMH | 259 LMH | 416 LMH |

Turning to FIG. 21D, illustrated is a graph 1630 of a plot of the volume experienced by each of the pod-type filter devices 1130A-1130D of the linear manifold 1100 in accordance with the manifold arrangement of experimental run 4 depicted in Table 1 above. More specifically, the linear manifold 1100 was evaluated where the main inlet conduit 1110 had an internal diameter of approximately 0.125 inches and the branch inlet conduits 1120A-1120D all had an internal diameter of approximately 0.0625 inches. In addition, each of the branch inlet conduits 1120A-1120D were equal in length. The pod-type filtration devices 1130A-1130D coupled to the branch inlet conduits 1120A-1120D were XOSP grade filters having a filter size or surface area of approximately 20 cm². Like that of the first through third experimental runs, a pump was used to flush deionized water through the linear manifold 1100 at a targeted flowrate of 300 LMH for at least 50 L/m², where the feed stream water was collected individually from the four outlet conduits 1140A-1140D into buckets with scales. In addition, the filter pressure and flush volume were continuously recorded by means of a data recorder. As depicted in the graph 1630 in FIG. 21D, which is a plot of the volume received by each of the pod-type filtration devices 1130A-1130D versus time for the arrangement of the linear manifold 1100 as depicted in Table 1 for the fourth experimental run, a negligible variation exists between the volume measured at each of the pod-type filtration devices 1130A-1130D. Moreover, as demonstrated in Table 5 below, which depicts the flowrates measured for each of the pod-type filtration devices 1130A-1130D, a negligible variation exists (e.g., approximately 21 LMH) between the fastest and slowest flowrates measured at the pod-type filtration devices 1130A-1130D. Thus, according to the data from the fourth experimental run, by further varying the inner diameters of the conduits as well as changing the pod-type filtration devices to a smaller scale filter device having tighter filter grades, a near uniform flow distribution of a non-plugging feed stream was achieved for the arrangement of the linear manifold 1100 depicted in Table 1 for the fourth experimental run.

TABLE 5
Experimental Run 4 Individual Flowrates

| 1130A | 1130B | 1130C | 1130D |
|---|---|---|---|
| 298 LMH | 277 LMH | 283 LMH | 280 LMH |

Turning to FIG. 21E, illustrated is a graph 1640 of a plot of the volume experienced by each of the pod-type filter devices 1440A-1440D of the bifurcated manifold 1400 in accordance with the manifold arrangement of experimental run 5 depicted in Table 1 above. More specifically, the bifurcated manifold 1400 was evaluated where the main inlet conduit 1410, the primary inlet branch conduit 1420, and the secondary inlet branch conduits 1430A-1430D all had an internal diameter of approximately 0.25 inches. In addition, each of the secondary inlet branch conduits 1430A-1430D were equal in length. The pod-type filtration devices 1440A-1440D coupled to the secondary inlet branch conduits 1430A-1430D were DOSP grade filters having a filter size or surface area of approximately 135 cm². Like that previously explained with the other experimental runs, a pump was used to flush deionized water through the bifurcated manifold 1400 at a targeted flowrate of 300 LMH for at least 50 L/m², where the feed stream water was collected individually from the four outlet conduits 1450A-1450D into buckets with scales. In addition, the filter pressure and flush volume were continuously recorded by means of a data recorder. As depicted in the graph 1640 in FIG. 21E, which is a plot of the volume received by each of the pod-type filtration devices 1440A-1440D versus time for the arrangement of the bifurcated manifold 1400 as depicted in Table 1 for the fifth experimental run, a large variation exists between the volume measured at each of the pod-type filtration devices 1440A-1440D. Moreover, as demonstrated in Table 6 below, which depicts the flowrates measured for each of the pod-type filtration devices 1440A-1440D, a large variation exists (e.g., approximately 210 LMH) between the fastest and slowest flowrates measured at the pod-type filtration devices 1440A-1440D. More specifically, the third pod-type filtration device 1440C received the largest flowrate and a greater share of the flow distribution of the non-plugging feed stream, while the first pod-type filtration device 1440A received the lowest flowrate and the lowest share of the flow distribution of the non-plugging feed stream. Thus, according to the data from the fifth experimental run, a near uniform flow distribution of a non-plugging feed stream could not be achieved for the arrangement of the bifurcated manifold 1400 depicted in Table 1 for the fifth experimental run.

TABLE 6
Experimental Run 5 Individual Flowrates

| 1440A | 1440B | 1440C | 1440D |
|---|---|---|---|
| 207 LMH | 336 LMH | 417 LMH | 234 LMH |

Turning to FIG. 21F, illustrated is a graph 1650 of a plot of the volume experienced by each of the pod-type filter devices 1440A-1440D of the bifurcated manifold 1400 in accordance with the manifold arrangement of experimental run 6 depicted in Table 1 above. More specifically, the bifurcated manifold 1400 was evaluated where the main inlet conduit 1410 had an internal diameter of approximately 0.25 inches, the primary inlet branch conduit 1420 had an internal diameter of approximately 0.125 inches, and the secondary inlet branch conduits 1430A-1430D all had an internal diameter of approximately 0.0625 inches. In addition, each of the secondary inlet branch conduits 1430A-1430D were equal in length. The pod-type filtration devices 1440A-1440D coupled to the secondary inlet branch conduits 1430A-1430D were DOSP grade filters having a filter size or surface area of approximately 135 cm². Like that previously explained with the fifth experimental run, a pump was used to flush deionized water through the bifurcated manifold 1400 at a targeted flowrate of 300 LMH for at least 50 L/m², where the feed stream water was collected individually from the four outlet conduits 1450A-1450D into buckets with scales. In addition, the filter pressure and flush volume were continuously recorded by means of a data recorder. As depicted in the graph 1650 in FIG. 21F, which is a plot of the volume received by each of the pod-type filtration devices 1440A-1440D versus time for the arrangement of the bifurcated manifold 1400 as depicted in Table 1 for the sixth experimental run, a negligible variation exists between the volume measured at each of the pod-type filtration devices 1440A-1440D. Moreover, as demonstrated in Table 7 below, which depicts the flowrates measured for each of the pod-type filtration devices 1440A-1440D, a negligible variation exists (e.g., approximately 40 LMH) between the fastest and slowest flowrates measured at the pod-type filtration devices 1440A-1440D. While the differences were negligible, the second and fourth pod-type filtration devices 1440B, 1440D, respectively, received the largest flowrate and a greater share of the flow distribution of the non-plugging feed stream, while the first pod-type filtration device 1440A received the lowest flowrate and the lowest share of the flow distribution of the non-plugging feed stream. Thus, according to the data from the sixth experimental run, a near uniform flow distribution of a non-plugging feed stream was achieved for the arrangement of the bifurcated manifold 1400 depicted in Table 1 for the sixth experimental run, where the internal diameters of the main inlet conduit 1410, the primary inlet branched conduit 1420, and the secondary inlet branch conduits 1430A-1430D are varied and halved at each successive conduit group (i.e., the internal diameter of the primary inlet branch conduit 1420 is halve of that of the main inlet conduit 1410, the internal diameters of the secondary inlet branch conduits 1430A-1430D are half of the that of the primary inlet branch conduit 1420).

TABLE 7
Experimental Run 6 Individual Flowrates

| 1440A | 1440B | 1440C | 1440D |
|---|---|---|---|
| 273 LMH | 313 LMH | 289 LMH | 313 LMH |

Turning to FIG. 21G, illustrated is a graph 1660 of a plot of the volume experienced by each of the pod-type filter devices 1440A-1440D of the bifurcated manifold 1400 in accordance with the manifold arrangement of experimental run 7 depicted in Table 1 above. More specifically, the bifurcated manifold 1400 was evaluated where the main inlet conduit 1410 had an internal diameter of approximately 0.25 inches, the primary inlet branch conduit 1420 had an internal diameter of approximately 0.125 inches, and the secondary inlet branch conduits 1430A-1430D all had an internal diameter of approximately 0.0625 inches. In addition, each of the secondary inlet branch conduits 1430A-1430D were equal in length. The pod-type filtration devices 1440A-1440D coupled to the secondary inlet branch conduits 1430A-1430D were 60HX grade filters having a filter size or surface area of approximately 135 cm². Like that previously explained with the fifth and sixth experimental runs, a pump was used to flush deionized water through the bifurcated manifold 1400 at a targeted flowrate of 300 LMH for at least 50 L/m², where the feed stream water was collected individually from the four outlet conduits 1450A-1450D into buckets with scales. In addition, the filter pressure and flush volume were continuously recorded by means of a data recorder. As depicted in the graph 1660 in FIG. 21G, which is a plot of the volume received by each of the pod-type filtration devices 1440A-1440D versus time for the arrangement of the bifurcated manifold 1400 as depicted in Table 1 for the seventh experimental run, a slight variation exists between the volume measured at each of the pod-type filtration devices 1440A-1440D. Moreover, as demonstrated in Table 7 below, which depicts the flowrates measured for each of the pod-type filtration devices 1440A-1440D, a slight variation exists (e.g., approximately 89 LMH) between the fastest and slowest flowrates measured at the pod-type filtration devices 1440A-1440D. While there was a slight variability in the measured volume and flowrate, the variability was less than that experienced in the first through third experimental runs of the linear manifold 1100. As depicted in the data, the fourth pod-type filtration device 1440D received the largest flowrate and a greater share of the flow distribution of the non-plugging feed stream, while the first pod-type filtration device 1440A received the lowest flowrate and the lowest share of the flow distribution of the non-plugging feed stream. Thus, according to the data from the seventh experimental run, the flow distribution of a non-plugging feed stream for the arrangement of the bifurcated manifold 1400 depicted in Table 1 for the seventh experimental run was more uniform than that of the first three experimental runs of the linear manifold 1100. The seventh experimental run, however, did not contain as uniform of a flow distribution as that of the sixth experimental run, where only difference between the two runs was the pod-type filtration devices 1440A-1440D utilized (e.g., DOSP filters vs. 60HX filters).

TABLE 8
Experimental Run 7 Individual Flowrates

| 1440A | 1440B | 1440C | 1440D |
|---|---|---|---|
| 255 LMH | 294 LMH | 324 LMH | 344 LMH |

Turning to FIG. 21H, illustrated is a graph 1670 of a plot of the volume experienced by each of the pod-type filter devices 1440A-1440D of the bifurcated manifold 1400 in accordance with the manifold arrangement of experimental run 8 depicted in Table 1 above. More specifically, the bifurcated manifold 1400 was evaluated where the main inlet conduit 1410 had an internal diameter of approximately 0.25 inches, the primary inlet branch conduit 1420 had an internal diameter of approximately 0.125 inches, and the secondary inlet branch conduits 1430A-1430D all had an internal diameter of approximately 0.0625 inches. In addition, each of the secondary inlet branch conduits 1430A-1430D were equal in length. The pod-type filtration devices 1440A-1440D coupled to the secondary inlet branch conduits 1430A-1430D were XOSP grade filters having a filter size or surface area of approximately 135 cm². Like that previously explained with the fifth through seventh experimental runs, a pump was used to flush deionized water through the bifurcated manifold 1400 at a targeted flowrate of 300 LMH for at least 50 L/m², where the feed stream water was collected individually from the four outlet conduits 1450A-1450D into buckets with scales. In addition, the filter pressure and flush volume were continuously recorded by means of a data recorder. As depicted in the graph 1670 in FIG. 21H, which is a plot of the volume received by each of the pod-type filtration devices 1440A-1440D versus time for the arrangement of the bifurcated manifold 1400 as depicted in Table 1 for the eighth experimental run, a negligible variation exists between the volume measured at each of the pod-type filtration devices 1440A-1440D. Moreover, as demonstrated in Table 9 below, which depicts the flowrates measured for each of the pod-type filtration devices 1440A-1440D, a negligible variation exists (e.g., approximately 52 LMH) between the fastest and slowest flowrates measured at the pod-type filtration devices 1440A-1440D. While the differences were negligible, the second pod-type filtration devices 1440B received the largest flowrate and a greater share of the flow distribution of the non-plugging feed stream, while the third pod-type filtration device 1440C received the lowest flowrate and the lowest share of the flow distribution of the non-plugging feed stream. Thus, according to the data from the eight experimental run, a near uniform flow distribution of a non-plugging feed stream was achieved for the arrangement of the bifurcated manifold 1400 depicted in Table 1 for the eighth experimental run, where the arrangement of the bifurcated manifold 1400 is the same as that of the sixth and seventh experimental runs except for the pod-type filtration devices 1440A-1440D (e.g., DOSP vs. 60HX vs. XOSP).

TABLE 9
Experimental Run 8 Individual Flowrates

| 1440A | 1440B | 1440C | 1440D |
|---|---|---|---|
| 283 LMH | 322 LMH | 270 LMH | 316 LMH |

Turning to FIG. 21I, illustrated is a graph 1680 of a plot of the volume experienced by each of the pod-type filter devices 1440A-1440D of the bifurcated manifold 1400 in accordance with the manifold arrangement of experimental run 9 depicted in Table 1 above. More specifically, the bifurcated manifold 1400 was evaluated where the main inlet conduit 1410 had an internal diameter of approximately 0.125 inches, the primary inlet branch conduit 1420 had an internal diameter of approximately 0.0625 inches, and the secondary inlet branch conduits 1430A-1430D all had an internal diameter of approximately 0.03125 inches. In addition, each of the secondary inlet branch conduits 1430A-1430D were equal in length. The pod-type filtration devices 1440A-1440D coupled to the secondary inlet branch conduits 1430A-1430D were XOSP grade filters having a filter size or surface area of approximately 20 cm². Like that previously explained with the fifth through eighth experimental runs, a pump was used to flush deionized water through the bifurcated manifold 1400 at a targeted flowrate of 300 LMH for at least 50 L/m², where the feed stream water was collected individually from the four outlet conduits 1450A-1450D into buckets with scales. In addition, the filter pressure and flush volume were continuously recorded by means of a data recorder. As depicted in the graph 1680 in FIG. 21I, which is a plot of the volume received by each of the pod-type filtration devices 1440A-1440D versus time for the arrangement of the bifurcated manifold 1400 as depicted in Table 1 for the ninth experimental run, a negligible variation exists between the volume measured at each of the pod-type filtration devices 1440A-1440D. Moreover, as demonstrated in Table 10 below, which depicts the flowrates measured for each of the pod-type filtration devices 1440A-1440D, a negligible variation exists (e.g., approximately 84 LMH) between the fastest and slowest flowrates measured at the pod-type filtration devices 1440A-1440D. While the differences were negligible, the fourth pod-type filtration devices 1440D received the largest flowrate and a greater share of the flow distribution of the non-plugging feed stream, while the third pod-type filtration device 1440C received the lowest flowrate and the lowest share of the flow distribution of the non-plugging feed stream. Thus, according to the data from the ninth experimental run, a near uniform flow distribution of a non-plugging feed stream was achieved for the arrangement of the bifurcated manifold 1400 depicted in Table 1 for the ninth experimental run. The ninth experimental run, in conjunction with the eighth experimental run, demonstrates that the bifurcated manifold 1400, having a variety of internal diameters as depicted in Table 1, promotes a near uniform flow distribution across different sized pod-type filtration devices 1440A-1440D of the same filter grade.

TABLE 10
Experimental Run 9 Individual Flowrates

| 1440A | 1440B | 1440C | 1440D |
|---|---|---|---|
| 319 LMH | 315 LMH | 295 LMH | 379 LMH |

Turning to FIG. 21J, illustrated is a graph 1690 of a plot of the volume experienced by each of the pod-type filter devices 1440A-1440D of the bifurcated manifold 1400 in accordance with the manifold arrangement of experimental run 10 depicted in Table 1 above. More specifically, the bifurcated manifold 1400 was evaluated where the main inlet conduit 1410 had an internal diameter of approximately 0.125 inches, the primary inlet branch conduit 1420 had an internal diameter of approximately 0.0625 inches, and the secondary inlet branch conduits 1430A-1430D all had an internal diameter of approximately 0.03125 inches. In addition, each of the secondary inlet branch conduits 1430A-1430D were equal in length. The pod-type filtration devices 1440A-1440D coupled to the secondary inlet branch conduits 1430A-1430D were DOSP grade filters having a filter size or surface area of approximately 20 cm². Like that previously explained with the fifth through ninth experimental runs, a pump was used to flush deionized water through the bifurcated manifold 1400 at a targeted flowrate of 300 LMH for at least 50 L/m², where the feed stream water was collected individually from the four outlet conduits 1450A-1450D into buckets with scales. In addition, the filter pressure and flush volume were continuously recorded by means of a data recorder. As depicted in the graph 1690 in FIG. 21J, which is a plot of the volume received by each of the pod-type filtration devices 1440A-1440D versus time for the arrangement of the bifurcated manifold 1400 as depicted in Table 1 for the tenth experimental run, a negligible variation exists between the volume measured at each of the pod-type filtration devices 1440A-1440D. Moreover, as demonstrated in Table 11 below, which depicts the flowrates measured for each of the pod-type filtration devices 1440A-1440D, a negligible variation exists (e.g., approximately 23 LMH) between the fastest and slowest flowrates measured at the pod-type filtration devices 1440A-1440D. While the differences were negligible, the second pod-type filtration device 1440B received the largest flowrate and a greater share of the flow distribution of the non-plugging feed stream, while the fourth pod-type filtration device 1440D received the lowest flowrate and the lowest share of the flow distribution of the non-plugging feed stream. Thus, according to the data from the tenth experimental run, a near uniform flow distribution of a non-plugging feed stream was achieved for the arrangement of the bifurcated manifold 1400 depicted in Table 1 for the tenth experimental run. The tenth experimental run, in conjunction with the fifth through ninth experimental runs, demonstrates that the bifurcated manifold 1400, having a variety of internal diameters as depicted in Table 1, promotes a near uniform flow distribution across different pod-type filtration devices 1440A-1440D (i.e., across both different filter grades and different filter sizes).

TABLE 11
Experimental Run 10 Individual Flowrates

| 1440A | 1440B | 1440C | 1440D |
|---|---|---|---|
| 321 LMH | 333 LMH | 315 LMH | 310 LMH |

Turning to FIGS. 22A-22K, and with continued reference to FIGS. 19B, 19C, 20A, and 20B, illustrated are graphs that depict the outcome of various experiments run on the linear Z-shaped manifold 1200, the linear U-shaped manifold 1300, and the bifurcated manifolds 1400, 1500 by flowing a plugging feed stream (e.g., diary whey, cell culture, and/or soy T) through the manifolds 1200, 1300, 1400, 1500. In total, and as depicted in Table 12 below, ten experiments were run using the manifolds 1200, 1300, 1400, 1500, where two of the experiments were run on the linear Z-shaped manifold 1200, one of the experiments was run on the linear U-shaped manifold 1300, one of the experiments was run on the bifurcated manifold 1400, and six of the experiments were run on the bifurcated manifold 1500. The internal diameters of the inlet conduits (i.e., including the branch inlet conduits) were varied in accordance with that depicted in Table 12, as well as the filter grade and filter size. The manifolding of the outlets was also varied, along with the type of feed stream that was pumped through the manifolds 1200, 1300, 1400, 1500. In addition, all outlet tubing for the experiments were sized by the filter type used, where the internal diameter of the outlet tubing for lab scale filters was approximately 0.25 inches (¼″) and the internal diameter of the outlet tubing for the micro scale filters was approximately 0.0625 inches ( 1/16″). As depicted in Table 12, three different filters media grades were tested, which are identical to those test in the first ten experiments and described above (e.g., DOSP filters, XOSP filters, and 60HX filters).

TABLE 12
Filtration Test Experiments with Plugging Feed Streams

| | | Main | Primary | Secondary | | | | |
|---|---|---|---|---|---|---|---|---|
| Experimental | Manifold | Inlet | Inlet | Inlet | Filter | Filter | | |
| Run | Type | Conduit ID | Branch ID | Branch ID | Grade | Size | Outlet | Feed Stream |
| 11 | Linear | ¼″ | ¼″ | N/A | D0SP | 135 LSP | Linear Z | Dairy Whey |
| 12 | Linear | ½″ | ¼″ | N/A | D0SP | 135 LSP | Linear Z | Dairy Whey |
| 13 | Linear | ¼″ | 1/16″ | N/A | X0SP | Micro 20 | Linear U | Dairy Whey |
| 14 | Bifurcated | ¼″ | ⅛″ | 1/16″ | D0SP | 135 LSP | Branched | Dairy Whey |
| 15 | Bifurcated | ¼″ | ⅛″ | 1/16″ | D0SP | 135 LSP | Branched | Cell Culture |
| 16 | Bifurcated | ¼″ | ⅛″ | 1/16″ | 60HX | 135 LSP | Branched | Soy T |
| 17 | Bifurcated | ¼″ | ⅛″ | 1/16″ | D0SP | 135 LSP | Individual | Dairy Whey |
| 18 | Bifurcated | ¼″ | ⅛″ | 1/16″ | X0SP | 135 LSP | Branched | Cell Culture |
| 19 | Bifurcated | ⅛″ | 1/16″ | 1/32″ | X0SP | Micro 20 | Branched | Cell Culture |
| 20 | Bifurcated | ⅛″ | 1/16″ | 1/32″ | D0SP | Micro 20 | Branched | Cell Culture |

Turning to FIG. 22A, illustrated is a graph 1700 of a plot of the throughput vs. pressure experienced by each of the pod-type filter devices 1230A-1230D of the linear Z-shaped manifold 1200 in accordance with the manifold arrangement of experimental run 11 depicted in Table 12. More specifically, the linear Z-shaped manifold 1200 was evaluated where the main inlet conduit 1210 and the inlet branch conduits 1220A-1220D all had an internal diameter of approximately 0.25 inches. The outlet branch conduits 1240A-1240D and the main outlet conduit 1250 had an internal diameter of approximately 0.25 inches. In addition, each of the inlet branch conduits 1220A-1220D were equal in length with one another, while each of the outlet branch conduits 1240A-1240D were equal in length with one another. The pod-type filtration devices 1230A-1230D coupled to the inlet branch conduits 1220A-1220D were DOSP grade filters having a filter size or surface area of approximately 135 cm². A pump was used to flush dairy whey having an approximate density of 30 grams per liter (g/L) through the linear Z-shaped manifold 1200 at a targeted flowrate of approximately 150 LMH. As the dairy whey was flushed through the linear Z-shaped manifold 1200, the pressures and the filtrate volumes experienced by the filter devices 1230A-1230D were continuously recorded by means of a data recorder with the aid of measurements taken by the inlet and outlet pressure transducers 1232A-1232D, 1246A-1246D, respectively. The filtrate from each of the pod-type filtration devices 1230A-1230D was collected at the second end 1254 or outlet of the linear Z-shaped manifold 1200. As depicted in the graph 1700 in FIG. 22A, which is a plot of the throughput achieved by each of the pod-type filtration devices 1230A-1230D versus pressure for the arrangement of the linear Z-shaped manifold 1200 as depicted in Table 12 for the eleventh experimental run, each of the four pod-type filtration devices 1230A-1230D had comparable pressure profiles for the respective inlets and outlets of the pod-type filtration devices 1230A-1230D. Moreover, each pod-type filtration devices 1230A-1230D delivered a filtration capacity of approximately 170 liters per square meter (L/m²). Thus, according to the data from the eleventh experiment, a near uniform depth filtration performance can be achieved using a plugging feed stream with the arrangement of the linear Z-shaped manifold 1200 depicted in Table 12 for the eleventh experimental run, where the main inlet conduit 1210 and the inlet branch conduits 1220A-1220D have equal internal diameters.

Turning to FIG. 22B, illustrated is a graph 1710 of a plot of the throughput vs. pressure experienced by each of the pod-type filter devices 1230A-1230D of the linear Z-shaped manifold 1200 in accordance with the manifold arrangement of experimental run 12 depicted in Table 12. More specifically, the linear Z-shaped manifold 1200 was evaluated where the main inlet conduit 1210 had an internal diameter of approximately 0.5 inches and each of the inlet branch conduits 1220A-1220D had an internal diameter of approximately 0.25 inches. Moreover, the outlet branch conduits 1240A-1240D and the main outlet conduit 1250 all had an internal diameter of approximately 0.25 inches. In addition, each of the inlet branch conduits 1220A-1220D were equal in length with one another, while each of the outlet branch conduits 1240A-1240D were equal in length with one another. The pod-type filtration devices 1230A-1230D coupled to the inlet branch conduits 1220A-1220D were DOSP grade filters having a filter size or surface area of approximately 135 cm². A pump was used to flush dairy whey having an approximate density of 30 g/L through the linear Z-shaped manifold 1200 at a targeted flowrate of approximately 150 LMH. As the dairy whey was flushed through the linear Z-shaped manifold 1200, the pressures and the filtrate volumes experienced by the filter devices 1230A-1230D were continuously recorded by means of a data recorder with the aid of measurements taken by the inlet and outlet pressure transducers 1232A-1232D, 1246A-1246D, respectively. The filtrate from each of the pod-type filtration devices 1230A-1230D was collected at the second end 1254 or outlet of the linear Z-shaped manifold 1200. As depicted in the graph 1710 in FIG. 22B, which is a plot of the throughput achieved by each of the pod-type filtration devices 1230A-1230D versus pressure for the arrangement of the linear Z-shaped manifold 1200 as depicted in Table 12 for the twelfth experimental run, each of the four pod-type filtration devices 1230A-1230D had comparable pressure profiles for the respective inlets and outlets of the pod-type filtration devices 1230A-1230D. Moreover, each pod-type filtration device 1230A-1230D delivered a filtration capacity of approximately 170 L/m². Thus, according to the data from the twelfth experiment, a near uniform depth filtration performance can be achieved using a plugging feed stream with the arrangement of the linear Z-shaped manifold 1200 depicted in Table 12 for the twelfth experimental run, where the main inlet conduit 1210 and the inlet branch conduits 1220A-1220D have differing internal diameters.

Turning to FIG. 22C, illustrated is a graph 1720 of a plot of the throughput vs. pressure experienced by each of the pod-type filter devices 1330A-1330D of the linear U-shaped manifold 1300 in accordance with the manifold arrangement at experimental run 13 depicted in Table 12. More specifically, the linear U-shaped manifold 1300 was evaluated where the main inlet conduit 1310 had an internal diameter of approximately 0.25 inches and each of the inlet branch conduits 1320A-1320D had an internal diameter of approximately 0.0625 inches. Moreover, the outlet branch conduits 1340A-1340D and the main outlet conduit 1350 all had an internal diameter of approximately 0.25 inches. In addition, each of the inlet branch conduits 1320A-1320D were equal in length with one another, while each of the outlet branch conduits 1340A-1340D were equal in length with one another. The pod-type filtration devices 1330A-1330D coupled to the inlet branch conduits 1320A-1320D were XOSP Micro20 grade filters having a filter size or surface area of approximately 20 cm². A pump was used to flush a dairy whey suspension having an approximate concentration of 5 g/L through the linear U-shaped manifold 1300 at a targeted flowrate of approximately 150 LMH. As the dairy whey was flushed through the linear U-shaped manifold 1300, the pressures and the filtrate volumes experienced by the filter devices 1330A-1330D were continuously recorded by means of a data recorder with the aid of measurements taken by the inlet and outlet pressure transducers 1332A-1332D, 1346A-1346D, respectively. The filtrate from each of the pod-type filtration devices 1330A-1330D was collected at the first end 1352 or outlet of the linear U-shaped manifold 1300. As depicted in the graph 1720 in FIG. 22C, which is a plot of the throughput achieved by each of the pod-type filtration devices 1330A-1330D versus pressure for the arrangement of the linear U-shaped manifold 1300 as depicted in Table 12 for the thirteenth experimental run, each of the four pod-type filtration devices 1330A-1330D had comparable pressure profiles for the respective inlets and outlets of the pod-type filtration devices 1330A-1330D. Moreover, each pod-type filtration device 1330A-1330D delivered a filtration capacity of approximately 55 L/m². Thus, according to the data from the thirteenth experiment, a near uniform depth filtration performance can be achieved using a plugging feed stream with the arrangement of the linear U-shaped manifold 1300 depicted in Table 12 for the thirteenth experimental run, where the main inlet conduit 1310 and the inlet branch conduits 1320A-1320D have differing internal diameters and where the pod-type filtration device is a micro 20 scale filter device.

Turning to FIG. 22D, illustrated is a graph 1730 of a plot of the throughput vs. pressure experienced by each of the pod-type filter devices 1540A-1540D of the bifurcated manifold 1500 in accordance with the manifold arrangement of experimental run 14 depicted in Table 12. More specifically, the bifurcated manifold 1500 was evaluated where the main inlet conduit 1510 had an internal diameter of approximately 0.25 inches, the primary inlet branch conduit 1520 had an internal diameter of approximately 0.125 inches, and each of the secondary inlet branch conduits 1530A-1530D had an internal diameter of approximately 0.0625 inches. Moreover, the secondary outlet branch conduits 1550A-1550D, the primary outlet branch conduit 1560, and the main outlet conduit 1570 all had an internal diameter of approximately 0.25 inches. In addition, each of the secondary inlet branch conduits 1530A-1530D were equal in length with one another, while each of the secondary outlet branch conduits 1550A-1550D were equal in length with one another. The pod-type filtration devices 1540A-1540D coupled to the secondary inlet branch conduits 1530A-1530D were DOSP grade filters having a filter size or surface area of approximately 135 cm². A pump was used to flush dairy whey having an approximate density of 30 g/L through the bifurcated manifold 1500 at a targeted flowrate of approximately 150 LMH. As the dairy whey was flushed through the bifurcated manifold 1500, the pressures and the filtrate volumes experienced by the filter devices 1540A-1540D were continuously recorded by means of a data recorder with the aid of measurements taken by the inlet and outlet pressure transducers 1542A-1542D, 1556A-1556D, respectively. The filtrate from each of the pod-type filtration devices 1540A-1540D was collected at the second end 1574 or outlet of the bifurcated manifold 1500. As depicted in the graph 1730 in FIG. 22D, which is a plot of the throughput achieved by each of the pod-type filtration devices 1540A-1540D versus pressure for the arrangement of the bifurcated manifold 1500 as depicted in Table 12 for the fourteenth experimental run, each of the four pod-type filtration devices 1540A-1540D had comparable pressure profiles for the respective inlets and outlets of the pod-type filtration devices 1540A-1540D. Moreover, each pod-type filtration device 1540A-1540D delivered a filtration capacity of approximately 175 L/m². Thus, according to the data from the fourteenth experiment, a near uniform depth filtration performance can be achieved using a plugging feed stream with the arrangement of the bifurcated manifold 1500 depicted in Table 12 for the fourteenth experimental run, where the various inlet conduits 1510, 1520, 1530A-1530D have differing internal diameters and where the pod-type filtration device is a DOSP media grade filter device.

Turning to FIG. 22E, illustrated is a graph 1740 of a plot of the throughput vs. pressure experienced by each of the pod-type filter devices 1540A-1540D of the bifurcated manifold 1500 in accordance with the manifold arrangement of experimental run 15 depicted in Table 12. The fifteenth experimental run was essentially a repeat of the fourteenth experimental run except that the plugging feed stream (e.g., diary whey) of the fourteenth experimental run was replaced with a cell culture feed stream (e.g., a mAb cell culture feed stream). More specifically, the bifurcated manifold 1500 was evaluated where the main inlet conduit 1510 had an internal diameter of approximately 0.25 inches, the primary inlet branch conduit 1520 had an internal diameter of approximately 0.125 inches, and each of the secondary inlet branch conduits 1530A-1530D had an internal diameter of approximately 0.0625 inches. Moreover, the secondary outlet branch conduits 1550A-1550D, the primary outlet branch conduit 1560, and the main outlet conduit 1570 all had an internal diameter of approximately 0.25 inches. In addition, each of the secondary inlet branch conduits 1530A-1530D were equal in length with one another, while each of the secondary outlet branch conduits 1550A-1550D were equal in length with one another. The pod-type filtration devices 1540A-1540D coupled to the secondary inlet branch conduits 1530A-1530D were DOSP grade filters having a filter size or surface area of approximately 135 cm². A pump was used to flush a mAb cell culture feed stream through the bifurcated manifold 1500 at a targeted flowrate of approximately 150 LMH. The mAb cell culture feed stream had a total cell density (TCD) of approximately 13.88e⁶ cells/mL, a viable cell density (VCD) of approximately 13.25 cells/mL, and a viability of approximately 95.4%. As the cell culture was flushed through the bifurcated manifold 1500, the pressures and the filtrate volumes experienced by the filter devices 1540A-1540D were continuously recorded by means of a data recorder with the aid of measurements taken by the inlet and outlet pressure transducers 1542A-1542D, 1556A-1556D, respectively.

The filtrate from each of the pod-type filtration devices 1540A-1540D was collected at the second end 1574 or outlet of the bifurcated manifold 1500. As depicted in the graph 1740 in FIG. 22E, which is a plot of the throughput achieved by each of the pod-type filtration devices 1540A-1540D versus pressure for the arrangement of the bifurcated manifold 1500 as depicted in Table 12 for the fifteenth experimental run, each of the four pod-type filtration devices 1540A-1540D had comparable pressure profiles for the respective inlets and outlets of the pod-type filtration devices 1540A-1540D. Moreover, each pod-type filtration device 1540A-1540D delivered a filtration capacity of approximately 250 L/m². Thus, according to the data from the fifteenth experiment, a near uniform depth filtration performance can be achieved using a cell culture feed stream with the arrangement of the bifurcated manifold 1500 depicted in Table 12 for the fifteenth experimental run, where the various inlet conduits 1510, 1520, 1530A-1530D have differing internal diameters and where the pod-type filtration device is a DOSP media grade filter device.

Turning to FIG. 22F, illustrated is a graph 1750 of a plot of the throughput vs. pressure experienced by each of the pod-type filter devices 1540A-1540D of the bifurcated manifold 1500 in accordance with the manifold arrangement of experimental run 16 depicted in Table 12. More specifically, the bifurcated manifold 1500 was evaluated where the main inlet conduit 1510 had an internal diameter of approximately 0.25 inches, the primary inlet branch conduit 1520 had an internal diameter of approximately 0.125 inches, and each of the secondary inlet branch conduits 1530A-1530D had an internal diameter of approximately 0.0625 inches. Moreover, the secondary outlet branch conduits 1550A-1550D, the primary outlet branch conduit 1560, and the main outlet conduit 1570 all had an internal diameter of approximately 0.25 inches. In addition, each of the secondary inlet branch conduits 1530A-1530D were equal in length with one another, while each of the secondary outlet branch conduits 1550A-1550D were equal in length with one another. The pod-type filtration devices 1540A-1540D coupled to the secondary inlet branch conduits 1530A-1530D were 60HX grade filters having a filter size or surface area of approximately 135 cm². A pump was used to flush a soy T feed stream having an approximate density of 50 g/L through the bifurcated manifold 1500 at a targeted flowrate of approximately 150 LMH. As the soy T feed stream was flushed through the bifurcated manifold 1500, the pressures and the filtrate volumes experienced by the filter devices 1540A-1540D were continuously recorded by means of a data recorder with the aid of measurements taken by the inlet and outlet pressure transducers 1542A-1542D, 1556A-1556D, respectively. The filtrate from each of the pod-type filtration devices 1540A-1540D was collected at the second end 1574 or outlet of the bifurcated manifold 1500. As depicted in the graph 1750 in FIG. 22F, which is a plot of the throughput achieved by each of the pod-type filtration devices 1540A-1540D versus pressure for the arrangement of the bifurcated manifold 1500 as depicted in Table 12 for the sixteenth experimental run, each of the four pod-type filtration devices 1540A-1540D had comparable pressure profiles for the respective inlets and outlets of the pod-type filtration devices 1540A-1540D. Moreover, each pod-type filtration device 1540A-1540D delivered a filtration capacity of approximately 100 L/m². Thus, according to the data from the sixteenth experiment, a near uniform depth filtration performance can be achieved using a plugging feed stream with the arrangement of the bifurcated manifold 1500 depicted in Table 12 for the sixteenth experimental run, where the various inlet conduits 1510, 1520, 1530A-1530D have differing internal diameters, where the pod-type filtration device is a 60HX media grade filter device, and where the plugging feed stream is soy T.

Turning to FIGS. 22G and 22H, illustrated are two graphs 1760, 1765 detailing the flow specifics experienced by each of the pod-type filter devices 1440A-1440D of the bifurcated manifold 1400 in accordance with the manifold arrangement of experimental run 17 depicted in Table 12. More specifically, the first graph 1760 plots the volume vs. time experienced by each of the pod-type filter devices 1440A-1440D of the bifurcated manifold 1400, while the second graph 1765 plots the throughput vs. pressure experienced by each of the pod-type filter devices 1440A-1440D of the bifurcated manifold 1400. With specific regard to the geometry of the bifurcated manifold 1400 of experimental run 17 depicted in Table 12, the main inlet conduit 1410 of the bifurcated manifold 1400 had an internal diameter of approximately 0.25 inches, the primary inlet branch conduit 1420 had an internal diameter of approximately 0.125 inches, and each of the secondary inlet branch conduits 1430A-1430D had an internal diameter of approximately 0.0625 inches. Moreover, each of the secondary inlet branch conduits 1430A-1430D were equal in length with one another. In addition, the outlets of the bifurcated manifold 1400 are not manifolded like that of the bifurcated manifolded 1500. Thus, extending from each of the pod-type filter devices 1440A-1440D is an outlet branch conduit 1450A-1450D, where the second ends 1454A-1454D, respectively, serve as individual outlets. The pod-type filtration devices 1440A-1440D coupled to the secondary inlet branch conduits 1430A-1430D were DOSP grade filters having a filter size or surface area of approximately 135 cm². A pump was used to flush dairy whey having an approximate density of 40 g/L through the bifurcated manifold 1400 at a targeted flowrate of approximately 150 LMH. As the dairy whey was flushed through the bifurcated manifold 1400, the pressures and the filtrate volumes experienced by the filter devices 1440A-1440D were continuously recorded by means of a data recorder with the aid of measurements taken by the inlet pressure transducers 1442A-1442D. The filtrate from each of the pod-type filtration devices 1440A-1440D was collected at each of the second ends 1454A-1454D of the outlet branch conduits 1450A-1450D, respectively of the bifurcated manifold 1400. As depicted in the graph 1760 in FIG. 22G, which is a plot of the flowrate experienced by each of the pod-type filtration devices 1440A-1440D for the arrangement of the bifurcated manifold 1400 as depicted in Table 12 for the seventeenth experimental run, each of the four pod-type filtration devices 1440A-1440D experienced comparable flowrates. Furthermore, as depicted in the graph 1765 in FIG. 22H, which is a plot of the throughput experienced by each of the pod-type filtration devices 1440A-1440D vs. pressure for the arrangement of the bifurcated manifold 1400 as depicted in Table 12 for the seventeenth experimental run, each of the four pod-type filtration devices 1440A-1440D experienced comparable pressure profiles for their respective inlets. Moreover, each pod-type filtration device 1440A-1440D delivered a filtration capacity of approximately 250 L/m². Thus, according to the data from the seventeenth experiment, a near uniform flow distribution and depth filtration performance can be achieved using a plugging feed stream with the arrangement of the bifurcated manifold 1400 depicted in Table 12 for the seventeenth experimental run, where the various inlet conduits 1410, 1420, 1430A-1430D have differing internal diameters, where the pod-type filtration device is a DOSP media grade filter device, and where the plugging feed stream is dairy whey.

Turning to FIG. 22I, illustrated is a graph 1770 of a plot of the throughput vs. pressure experienced by each of the pod-type filter devices 1540A-1540D of the bifurcated manifold 1500 in accordance with the manifold arrangement of experimental run 18 depicted in Table 12. The eighteenth experimental run was essentially a repeat of the fifteenth experimental run except that DOSP media grade filter devices were replaced with tighter media grade XOSP filter devices. More specifically, the bifurcated manifold 1500 was evaluated where the main inlet conduit 1510 had an internal diameter of approximately 0.25 inches, the primary inlet branch conduit 1520 had an internal diameter of approximately 0.125 inches, and each of the secondary inlet branch conduits 1530A-1530D had an internal diameter of approximately 0.0625 inches.

Moreover, the secondary outlet branch conduits 1550A-1550D, the primary outlet branch conduit 1560, and the main outlet conduit 1570 all had an internal diameter of approximately 0.25 inches. In addition, each of the secondary inlet branch conduits 1530A-1530D were equal in length with one another, while each of the secondary outlet branch conduits 1550A-1550D were equal in length with one another. The pod-type filtration devices 1540A-1540D coupled to the secondary inlet branch conduits 1530A-1530D were XOSP grade filters having a filter size or surface area of approximately 135 cm². A pump was used to flush a mAb cell culture feed stream through the bifurcated manifold 1500 at a targeted flowrate of approximately 150 LMH. The mAb cell culture feed stream had a TCD of approximately 37.13e⁶ cells/mL, a VCD of approximately 23.95e⁶ cells/mL, and a viability of approximately 64.5%. The cell culture was centrifuged to a low turbidity prior to the experimental run. As the cell culture was flushed through the bifurcated manifold 1500, the pressures and the filtrate volumes experienced by the filter devices 1540A-1540D were continuously recorded by means of a data recorder with the aid of measurements taken by the inlet and outlet pressure transducers 1542A-1542D, 1556A-1556D, respectively. The filtrate from each of the pod-type filtration devices 1540A-1540D was collected at the second end 1574 or outlet of the bifurcated manifold 1500. As depicted in the graph 1770 in FIG. 22I, which is a plot of the throughput achieved by each of the pod-type filtration devices 1540A-1540D versus pressure for the arrangement of the bifurcated manifold 1500 as depicted in Table 12 for the eighteenth experimental run, each of the four pod-type filtration devices 1540A-1540D had comparable pressure profiles for the respective inlets and outlets of the pod-type filtration devices 1540A-1540D. Moreover, each pod-type filtration device 1540A-1540D delivered a filtration capacity of approximately 500 L/m² and did not build pressure. Each of the pod-type filtration devices 1540A-1540D were within 10 nephelometric turbidity units (NTU) of one another when measured individually, which demonstrates that each branch (combination of secondary inlet branch conduit 1530A-1530D and pod-type filtration devices 1540A-1540D, respectively) of the manifold 1500 performed equally. Thus, according to the data from the eighteenth experiment, a near uniform depth filtration performance can be achieved using a cell culture feed stream with the arrangement of the bifurcated manifold 1500 depicted in Table 12 for the eighteenth experimental run, where the various inlet conduits 1510, 1520, 1530A-1530D have differing internal diameters and where the pod-type filtration device is an XOSP media grade filter device.

Turning to FIG. 22J, illustrated is a graph 1780 of a plot of the throughput vs. pressure experienced by each of the pod-type filter devices 1540A-1540D of the bifurcated manifold 1500 in accordance with the manifold arrangement of experimental run 19 depicted in Table 12. The nineteenth experimental run was essentially a repeat of the eighteenth experimental run except that lab scale XOSP media grade filter devices were scaled down to XOSP micro 20 filter devices, and the internal diameters of the conduits were downsized accordingly. More specifically, the bifurcated manifold 1500 was evaluated where the main inlet conduit 1510 had an internal diameter of approximately 0.125 inches, the primary inlet branch conduit 1520 had an internal diameter of approximately 0.0625 inches, and each of the secondary inlet branch conduits 1530A-1530D had an internal diameter of approximately 0.03125 inches. Moreover, the secondary outlet branch conduits 1550A-1550D, the primary outlet branch conduit 1560, and the main outlet conduit 1570 all had an internal diameter of approximately 0.25 inches. In addition, each of the secondary inlet branch conduits 1530A-1530D were equal in length with one another, while each of the secondary outlet branch conduits 1550A-1550D were equal in length with one another. The pod-type filtration devices 1540A-1540D coupled to the secondary inlet branch conduits 1530A-1530D were XOSP grade filters having a filter size or surface area of approximately 20 cm². A pump was used to flush a mAb cell culture feed stream through the bifurcated manifold 1500 at a targeted flowrate of approximately 150 LMH. The mAb cell culture feed stream had a TCD of approximately 37.13e⁶ cells/mL, a VCD of approximately 23.95e⁶ cells/mL, and a viability of approximately 64.5%. The cell culture was centrifuged to a low turbidity prior to the experimental run. As the cell culture was flushed through the bifurcated manifold 1500, the pressures and the filtrate volumes experienced by the filter devices 1540A-1540D were continuously recorded by means of a data recorder with the aid of measurements taken by the inlet and outlet pressure transducers 1542A-1542D, 1556A-1556D, respectively. The filtrate from each of the pod-type filtration devices 1540A-1540D was collected at the second end 1574 or outlet of the bifurcated manifold 1500. As depicted in the graph 1780 in FIG. 22J, which is a plot of the throughput achieved by each of the pod-type filtration devices 1540A-1540D versus pressure for the arrangement of the bifurcated manifold 1500 as depicted in Table 12 for the nineteenth experimental run, each of the four pod-type filtration devices 1540A-1540D had comparable pressure profiles for the respective inlets and outlets of the pod-type filtration devices 1540A-1540D. Moreover, each pod-type filtration device 1540A-1540D delivered a filtration capacity of approximately 950 L/m². Thus, according to the data from the nineteenth experiment, a near uniform depth filtration performance can be achieved using a cell culture feed stream with the arrangement of the bifurcated manifold 1500 depicted in Table 12 for the nineteenth experimental run, where the various inlet conduits 1510, 1520, 1530A-1530D have differing internal diameters and where the pod-type filtration device is a micro 20 XOSP media grade filter device.

Turning to FIG. 22K, illustrated is a graph 1790 of a plot of the throughput vs. pressure experienced by each of the pod-type filter devices 1540A-1540D of the bifurcated manifold 1500 in accordance with the manifold arrangement of experimental run 20 depicted in Table 12. The twentieth experimental run was essentially a repeat of the nineteenth experimental run except that micro scale XOSP media grade filter devices were swapped for DOSP micro 20 filter devices. Like the bifurcated manifold 1500 of the nineteenth experiment, the bifurcated manifold 1500 of the twentieth experiment had a main inlet conduit 1510 having an internal diameter of approximately 0.125 inches, a primary inlet branch conduit 1520 having an internal diameter of approximately 0.0625 inches, and secondary inlet branch conduits 1530A-1530D each having an internal diameter of approximately 0.03125 inches. Moreover, the secondary outlet branch conduits 1550A-1550D, the primary outlet branch conduit 1560, and the main outlet conduit 1570 all had an internal diameter of approximately 0.25 inches. In addition, each of the secondary inlet branch conduits 1530A-1530D were equal in length with one another, while each of the secondary outlet branch conduits 1550A-1550D were equal in length with one another. The pod-type filtration devices 1540A-1540D coupled to the secondary inlet branch conduits 1530A-1530D were DOSP grade filters having a filter size or surface area of approximately 20 cm². A pump was used to flush a mAb cell culture feed stream through the bifurcated manifold 1500 at a targeted flowrate of approximately 150 LMH. The mAb cell culture feed stream had a TCD of approximately 37.13e⁶ cells/mL, a VCD of approximately 23.95e⁶ cells/mL, and a viability of approximately 64.5%. The cell culture was centrifuged to a low turbidity prior to the experimental run. As the cell culture was flushed through the bifurcated manifold 1500, the pressures and the filtrate volumes experienced by the filter devices 1540A-1540D were continuously recorded by means of a data recorder with the aid of measurements taken by the inlet and outlet pressure transducers 1542A-1542D, 1556A-1556D, respectively. The filtrate from each of the pod-type filtration devices 1540A-1540D was collected at the second end 1574 or outlet of the bifurcated manifold 1500. As depicted in the graph 1790 in FIG. 22K, which is a plot of the throughput achieved by each of the pod-type filtration devices 1540A-1540D versus pressure for the arrangement of the bifurcated manifold 1500 as depicted in Table 12 for the twentieth experimental run, each of the four pod-type filtration devices 1540A-1540D had comparable pressure profiles for the respective inlets and outlets of the pod-type filtration devices 1540A-1540D. Moreover, each pod-type filtration device 1540A-1540D delivered a filtration capacity of approximately 650 L/m². Thus, according to the data from the twentieth experiment, a near uniform depth filtration performance can be achieved using a cell culture feed stream with the arrangement of the bifurcated manifold 1500 depicted in Table 12 for the twentieth experimental run, where the various inlet conduits 1510, 1520, 1530A-1530D have differing internal diameters and where the pod-type filtration device is a micro 20 DOSP media grade filter device.

While the apparatuses presented herein have been illustrated and described in detail and with reference to specific embodiments thereof, it is nevertheless not intended to be limited to the details shown, since it will be apparent that various modifications (e.g., various different types of filter devices) and structural changes may be made therein without departing from the scope of the inventions and within the scope and range of equivalents of the claims.

In addition, various features from one of the embodiments may be incorporated into another of the embodiments. That is, it is believed that the disclosure set forth above encompasses multiple distinct inventions with independent utility. While each of these inventions has been disclosed in a preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Accordingly, it is appropriate that the appended claims be construed broadly and in a manner consistent with the scope of the disclosure as set forth in the following claims.

It is also to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment of the invention. Additionally, it is also to be understood that the components of the apparatuses described herein, the heat extraction assembly described herein, or portions thereof may be fabricated from any suitable material or combination of materials, such as, but not limited to, plastic or metals (e.g., copper, bronze, aluminum, steel, etc.), as well as derivatives thereof, and combinations thereof. In addition, it is further to be understood that the steps of the methods described herein may be performed in any order or in any suitable manner.

Finally, when used herein, the term “comprises” and its derivations (such as “comprising”, etc.) should not be understood in an excluding sense, that is, these terms should not be interpreted as excluding the possibility that what is described and defined may include further elements, steps, etc. Similarly, where any description recites “a” or “a first” element or the equivalent thereof, such disclosure should be understood to include incorporation of one or more such elements, neither requiring nor excluding two or more such elements. Meanwhile, when used herein, the term “approximately” and terms of its family (such as “approximate”, etc.) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies, etc. The same applies to the terms “about”, “around”, “generally”, and “substantially.”

What is claimed is:

1. A flow manifold for a bioprocessing device, the flow manifold comprising:
a main conduit; and
a plurality of branch conduits in fluid communication with the main conduit, each branch conduit including a flow restrictor configured to evenly distribute a fluid flowing through the flow manifold among the plurality of branch conduits.

2. The flow manifold of claim 1, wherein the flow restrictor is a tubing with a first internal diameter that is smaller than a second internal diameter of the main conduit and a third internal diameter of each of the plurality of branch conduits, a pinch clamp, or an in-line orifice flow restrictor.

3. The flow manifold of claim 1, further comprising:
a filter coupled to each of the plurality of branch conduits, wherein each filter has a first flow resistance that is less than a second flow resistance of each of the flow restrictors.

4. The flow manifold of claim 3, wherein the second flow resistance is at least three times larger than the first flow resistance.

5. The flow manifold of claim 3, wherein each of the filters have similar media grades.

6. The flow manifold of claim 3, wherein each of the filters have different media grades.

7. The flow manifold of claim 1, wherein the flow manifold is a linear Z-shaped manifold.

8. The flow manifold of claim 1, wherein the flow manifold is a linear U-shaped manifold.

9. The flow manifold of claim 1, wherein the flow manifold is a bifurcated manifold, the bifurcated manifold having a primary branch conduit coupled to the main conduit, and wherein the plurality of branch conduits are a plurality of secondary branch conduits coupled to the primary branch conduit.

10. A flow manifold for a bioprocessing device, the flow manifold comprising:
a plurality of conduits that collectively form a plurality of flow pathways, at least one conduit of the plurality of conduits having a first flow resistance; and
a plurality of filter devices, each filter device being coupled to a respective conduit, each filter device having a second flow resistance that is less than the first flow resistance, wherein the flow manifold is configured to equally distribute a fluid flowing through the flow manifold.

11. The flow manifold of claim 10, wherein each of the filter devices is connected to the plurality of conduits via at least one hose barb, and wherein the at least one hose barb is equipped with a flow restrictor disposed within an internal conduit of the hose barb.

12. The flow manifold of claim 10, the plurality of conduits comprise:
a main conduit having a first internal diameter;
a primary branch conduit coupled to the main conduit and having a second internal diameter that is less than the first internal diameter; and
a plurality of secondary branch conduits coupled to the primary branch conduit, each of the plurality of secondary branch conduits having a third internal diameter that is less than the first internal diameter and the second internal diameter, wherein each of the filter devices of the plurality of filter devices is coupled to a respective secondary branch conduit of the plurality of secondary branch conduits.

13. The flow manifold of claim 10, the plurality of conduits comprise:
a main conduit having a first internal diameter; and
a plurality of branch conduits coupled to the main conduit, each of the plurality of branch conduits having a second internal diameter that is less than the first internal diameter, wherein each of the filter devices of the plurality of filter devices is coupled to a respective branch conduit of the plurality of branch conduits.

14. The flow manifold of claim 10, wherein each of the plurality of filter devices are of similar media grades having the second flow resistance.

15. The flow manifold of claim 10, wherein a first subset of the plurality of filter devices are of a first media grade having the second flow resistance, and a second subset of the plurality of filter devices are of a second media grade having a third flow resistance that is greater than the second flow resistance but less than the first flow resistance.

16. The flow manifold of claim 10, the plurality of conduits comprise:
a main conduit;
a plurality of branch conduits coupled to the main conduit; and
a plurality of flow restrictor conduits, where each flow restrictor conduit of the plurality of flow restrictor conduits is coupled to a respective branch conduit of the plurality of branch conduits, each of the plurality of flow restrictor conduits being equipped with a flow restrictor that imparts the first flow resistance to a respective flow restrictor conduit of the plurality of flow restrictor conduits, wherein each branch conduit of the plurality of branch conduits has a third flow resistance that is less than the first flow resistance and the second flow resistance, and wherein the plurality of filter devices are coupled to the plurality of branch conduits downstream from the flow restrictor conduits.

17. A flow manifold for a bioprocessing device, the flow manifold comprising:
a main conduit;
a plurality of filter branch conduits in fluid communication with the main conduit, each filter branch conduit including a filter device;
a plurality of flow restrictor conduits each including a flow restrictor, wherein each flow restrictor conduit has a first end and an opposite second end, the first and second ends being coupled to a respective filter branch conduit upstream of the filter device, wherein the plurality of flow restrictors are configured to evenly distribute a fluid flowing through the flow manifold among the plurality of filter branch conduits; and
a plurality of flow regulators, where each flow regulator is operatively coupled to a filter branch conduit of the plurality of filter branch conduits, wherein each flow regulator is configured to regulate the flow of the fluid through either the respective flow restrictor conduit or divert the flow of the fluid from the flow restrictor conduit.

18. The flow manifold of claim 17, wherein each filter device has a first flow resistance that is greater than a second flow resistance of each of the flow restrictors.

19. The flow manifold of claim 17, wherein the flow manifold is a linear Z-shaped manifold or a linear U-shaped manifold.

20. The flow manifold of claim 17, wherein the flow manifold is a bifurcated manifold.
